(19) United States
(12) Patent Application Publication
(10) Pub. No.: US 2010/0286490 A1
(43) Pub. Date: Nov. 11, 2010

(54) INTERACTIVE PATIENT MONITORING SYSTEM USING SPEECH RECOGNITION

(75) Inventor: Dennis A. Koverzin, Winnipeg (CA)

Correspondence Address: FISH & RICHARDSON P.C., PO BOX 1022, MINNEAPOLIS, MN, 55440-1022, US

(73) Assignee: IQ LIFE, INC., Winnipeg, MB (CA)

(21) Appl. No.: 12/297,634

(22) PCT Filed: Apr. 20, 2007
(86) PCT No.: PCT/CA2007/000674

§ 371 Date: Feb. 18, 2009

Related U.S. Application Data
(60) Provisional application No. 60/793,097, filed on Apr. 20, 2006.

(51) Int. Cl. A61B 5/00; G10L 15/00; G08B 23/00

(52) U.S. Cl. 600/301; 704/231; 340/573.1; 704/E15.039

(57) ABSTRACT

Systems, methods and techniques are described for monitoring a subject. The subject's safety, health and wellbeing can be monitoring using a system that receives input indicating the subject's status. The system can verbally interact with the subject to obtain information on the subject's status. The words used by the subject or the quality of the subject's response can be used to decide whether to contact emergency services to assist the subject.

### BACKGROUND

This invention relates to emergency monitors.

Many people live with poor health conditions such as a weak heart, diabetes, or age-related reduced strength. These people are at risk, to one degree or another, of experiencing a sudden health emergency, such as a heart attack or stroke. These people are also at risk of other types of sudden emergencies, such as bad falls.

The situation can be dangerous if the person lives alone, or is frequently alone. There are several reasons for this. First, a sudden health emergency (SHE) may occur so rapidly that the person becomes incapacitated before having a chance to call for help. This can occur if the SHE results in the rapid occurrence of unconsciousness, paralysis, extreme pain, deterioration of mental capacity (confusion), and other debilitating conditions. And because the person is alone, there is no one to observe the situation and to call for help.

Secondly, the person may be alone, and may begin experiencing the early warning signs of an SHE, such as a stroke or heart attack. Even though he or she sense a poor condition, he or she may not do anything about it initially. There are several reasons why this may happen. The person may, mistakenly, feel that the condition is not serious. Or the person may decide to wait awhile to see if the condition gets worse. Or the person may be uncertain as what to do, and so do nothing. By not taking action, the early warning signs can develop into a full-fledged SHE. It is thought that the chances of surviving an SHE, such as a heart attack, are greatly improved if treatment begins within an hour of onset of the SHE.

Thirdly, the person may exhibit the early warning signs of an SHE, but may not be aware of them. For example, the person may not sense that they have a droopy face, one of the early warning signs of a stroke. This could happen if the sign was so small that the person did not notice it, if the person did not consciously monitor her/himself for early warning signs on an on-going basis, or if the person was too busy to notice. As above, by not taking action, the early warning signs can develop into a full-fledged SHE.

If a person experiences an SHE, the person, or someone near the person, needs to quickly call emergency response personnel, or someone else who can help. An ambulance will be able to get to the person in short time, and will rush the person to a hospital for treatment. For example, if a person has a stroke, emergency response personnel or hospital staff may administer a clot-busting drug to the person, which could reduce potential damage to the brain. But this must be done within hours for the best chance of success.

### SUMMARY

In general, in one aspect, a method of monitoring a subject is described. The method includes initiating computer generated verbal interaction with the subject, including synthesizing speech to elicit a verbal response from the subject. Digitized sound is received from a monitor configured to receive verbal responses from the subject. Speech recognition is performed on the digitized sound to generate corresponding text. A subject's quality of responsiveness to the synthesized speech is determined with a computer. Whether to contact a predetermined contact for the subject is determined after determining the quality of the responsiveness.

In another aspect, a method of monitoring a subject is described. A computer generated verbal interaction with the subject is initiated, including synthesizing speech to elicit a verbal response from the subject. A response from the subject is awaited for a predetermined time. Whether the subject has responded within the predetermined time is determined. If the subject has not responded, emergency services are automatically contacted.

In another aspect, a method of monitoring a subject is described. The subject receives a digitized sound. The invention performs speech recognition on the digitized sound. The computer uses the digitized sound to determine whether the subject has verbally responded to a computer generated verbal query. If the subject has responded, the computer determines whether the subject has delayed in responding beyond a predetermined threshold time, the subject has provided a non-valid response, the subject has responded with unclear speech, the subject has provided a response using non-programmed vocabulary, or the subject has provided an expected response. Based on the subject's response, the determination is made either to submit to the subject a subsequent computer generated verbal question in a script, including synthesizing speech to elicit a verbal response from the subject or to request emergency services for the subject.

In another aspect, a method of monitoring a subject is described. Computer generated verbal interaction is initiated with the subject, including synthesizing speech to elicit a verbal response from the subject. A first statement or question from a script is submitted, wherein the first statement or question is submitted as a computer generated verbal statement or question. A digitized sound in response to the first question or statement is received from the subject. A speech recognition is performed on the digitized sound to generate text. A predetermined length of time is awaited. When the predetermined length of time has elapsed, a second computer generated verbal interaction with the subject is initiated, including synthesizing speech to elicit a verbal response from the subject. After initiating the second computer generated verbal interaction with the subject, a second statement or question is submitted to the subject.

In another aspect, a method of determining whether an emergency has occurred is described. A computer uses speech recognition to detect a keyword emitted by the subject. The keyword emitted by the subject initiates a request for emergency services.

In another aspect, a method of monitoring a patient is described. A first computer generated verbal interaction is initiated with the subject, including synthesizing speech to elicit a verbal response from the subject. A question is submitted to the subject, wherein the question is submitted as synthesized speech. A digitized first response to the question is received from the subject. Speech recognition is performed on the digitized first response. From the first response or the text, a baseline for the subject is determined. The baseline is stored in computer readable memory. A second computer generated verbal interaction with the subject is initiated, including synthesizing speech to elicit a verbal response from the subject. After initiating the second computer generated verbal interaction with the subject, a question is submitted to the subject, wherein the question is submitted as synthesized speech. A digitized second response to the question is received from the subject. Speech recognition is performed on the digitized second response to generate text. The second response or the text is compared to the baseline to determine a delta and whether to initiate emergency services is determined based on the delta.

In another aspect, a method of monitoring a subject is described. The method comprises initiating a computer generated verbal interaction with the subject, including synthesizing speech to elicit a verbal response from the subject. A question is submitted to the subject, wherein the question is submitted as synthesized speech. A digitized response to the question is received from the subject. Speech recognition is performed on the digitized response. Whether the subject has responded with an expected response is determined from the text. If the subject has not answered with an expected response, a predetermined contact is alerted.

In yet another aspect, a method of monitoring a subject is described. The method comprises detecting a trigger condition. A computer initiates a generated verbal interaction with the subject, including synthesizing speech to elicit a verbal response from the subject. If the subject responds, a digitized sound is received from a monitor configured to receive verbal responses from the subject. Speech recognition is performed on any digitized sound received from the subject to generate corresponding text. A computer determines either a quality of responsiveness of the subject to the synthesized speech or a meaning of the text and determines from the quality of responsiveness of the subject or the meaning of the text whether to request emergency services.

In yet another aspect, a method of simulating human interaction with a subject is described. The method comprises initiating a computer generated verbal interaction with the subject, including synthesizing speech to elicit a verbal response from the subject. A question from a first script is submitted to a subject, wherein the question is submitted as a computer generated verbal question or statement. A trigger event is detected. In response to detecting the trigger event, a second script is selected and a question from the second script is submitted to the subject, wherein the question is submitted as a computer generated verbal question or statement.

In another aspect, a method of simulating human interaction with a subject is described. The method comprises initiating a computer generated verbal interaction with the subject, including synthesizing speech to elicit a verbal response from the subject. A first question from a script is submitted to the subject, wherein the question is submitted as a computer generated verbal question, and the script has a first question, a second question and a third question to be presented to the subject in chronological order. A digitized sound in response to the first question is received from the subject. Speech recognition is performed on the digitized sound to generate text. A response to the second question from the script is determined to be stored in memory. The third question from the script is submitted to the subject without first submitting the second question to the subject and the question is submitted as a computer generated verbal question.

In another aspect, a method of monitoring a subject is described. The method includes initiating a computer generated verbal interaction with the subject, including generating synthesized speech having a question to elicit a verbal response from the subject. A digitized response to the question from the subject is received from a monitor configured to receive verbal responses from the subject. Speech recognition is performed on the digitized response to create text. From the text it is determined whether the subject requires emergency services. If the subject requires emergency services, a predetermined contact is alerted.

Systems, devices and computer program products to perform the method are described as well.

Embodiments of the invention can include one or more of the following features. Whether to contact a predetermined contact for the subject can include basing the determination on the quality of the responsiveness. The quality of responsiveness may be one of delayed, valid or invalid. An invalid response may be a response that can include unrecognized vocabulary, at least a phrase that is not anticipated or an unparseable response. A plurality of anticipated responses to the synthesized speech can be anticipated, and the speech recognition can recognize a word that is not in the plurality of anticipated responses. A determination may be made to contact a predetermined contact when the quality of responsiveness may be delayed or invalid. After determining with a computer the quality of the responsiveness, additional synthesized speech can be generated to elicit a further verbal response from the subject, wherein the additional synthesized speech can pose a question to the subject regarding a safety or health status of the subject; a response to the question regarding the safety or health status of subject can be received; speech recognition can be performed on the response to generate corresponding subsequent text; and whether to contact a predetermined contact may be determined based on the subsequent text. The digitized sound may be stored in memory. The digitized sound that may be stored in memory can be time stamped. The text may be stored in memory and optionally time stamped. A trigger event may be received, wherein the trigger event can initiate the computer generated verbal interaction with the subject. The trigger event may be a physiological parameter value that may be outside a predetermined range, a predetermined sound or a lack of a predetermined sound, a non-verbal vocal sound made by the subject or an environmental sound in the vicinity of the subject or one of a preset time, determining that the subject has not spoken for a predetermined time, or a response from a subject during a conversation or a completion of a script. The trigger event may be a predetermined image or a lack of a predetermined image. A trigger event can include receiving digitized sound from the subject, receiving a triggering digitized sound from the monitor configured to receive verbal responses from the subject, and performing speech recognition on the triggering digitized sound to generate corresponding triggering text. The triggering text may be the word emergency or the word help. A trigger event can include receiving a keyword that is a predefined word. The predetermined contact may be an emergency service. Determining in the computer whether to contact a predetermined contact can include determining whether to contact a predetermined contact based on the text. The predetermined contact may be emergency services.

Determining the quality of responsiveness of the subject can include determining that the response is a valid response, the method further comprising determining that the text indicates that the subject has requested assistance; and because the subject has requested assistance, determining to contact a predetermined contact includes determining to contact emergency services. Determining from the quality of responsiveness of the subject whether to request emergency services can include determining that the response is an invalid response indicating that the subject may be in need of emergency assistance; and because the subject has requested assistance, determining to contact a predetermined contact includes determining to contact emergency services. Determining from the quality of responsiveness of the subject whether to request emergency services can include determining that a delay of the response is greater than a predetermined delay threshold and because the delay may be greater than the threshold, determining to contact emergency services. Determining from the quality of responsiveness of the subject can include determining that the response may be an invalid response indicating that the subject may be in danger of physical harm. The method can further comprise receiving secondary signal, including one of a physiological parameter values, a recognized sound-based event, or a recognized image-based events and using the received signal in conjunction with the quality of responsiveness to determine whether to contact emergency services as the predetermined contact.

A response from the subject can include a verbal response or a non-verbal sound. Submitting to the subject a subsequent computer generated verbal question can include submitting a question regarding a safety or health status of the subject. The script may be a script of questions related to detecting a heart attack, a stroke, cardiac arrest or a fall. The script may be a script of questions related to detecting whether the subject may be in physical danger.

A digitized sound in response to the second question can be received from the subject. Speech recognition can be performed on the digitized sound in response to the second question and the digitized sound in response to the second question can be compared with the digitized sound that is stored in memory. The digitized sound or text generated from the digitized sound can be transmitted to a control center after determining in a computer to request emergency services. Speech recognition can be performed on the digitized sound to create a digitized response, the method can further comprise performing speech recognition on the digitized sound, determining from the digitized response that the subject is experiencing an event and assigning a value to the event, such as pain, where the value can be one of none, little, moderate or severe. The method can comprise after submitting to the subject a first question from a script, re-submitting to the subject the first question from the script and providing the subject with a list of acceptable replies to the first question.

Embodiments of the invention can includes the following features. The keyword can be emergency or help. The method of monitoring may be used to determine that the subject may have lost ability to understand or to monitor a mental status of the subject. The method can comprise retrieving emergency contact information from a database and using the emergency contact information to send a digital alert to the predetermined contact.

The trigger condition may be one of digitized sound received from the subject, a digitized sound captured in the subject's environment, or a digital image of the subject falling or not moving. The trigger condition may be a value of a physiological parameter that may be outside of a predetermined range. The physiological parameter may be one of an ECG signal, a blood oxygen saturation level, blood pressure, acceleration downwards, blood glucose, heart rate, heart beat sound or temperature.

Embodiments of the invention can include one or more of the following features. The detection of the trigger event can include receiving a verbal response from the subject in digital form, performing speech recognition on the verbal response in digital form to generate text and determining from the text that the response indicates that the subject is experiencing an emergency. The trigger event may be a keyword spoken by the client, a physiological parameter value that is outside a predetermined range, a predetermined sound or a lack of a predetermined sound, a non-verbal vocal sound made by the subject or an environmental sound in the vicinity of the subject or one of a preset time, determining that the subject may have not spoken for a predetermined time, or a response from a subject during a conversation or a completion of a script. The trigger event may be a predetermined image or a lack of a predetermined image. The emergency be detected may be a health emergency, such as heart attack, stroke, cardiac arrest, loss of understanding, loss of motion, loss of responsiveness, or a fall. The second script can include questions to verify whether the subject is experiencing heart attack, stroke, cardiac arrest, loss of understanding, loss of motion, loss of responsiveness, a fall or an early warning sign of the health emergency. Questions from the first script can be asked after questions from a second script interrupt the first script. Where the first script has at least one group of questions, the group of questions including a first question and a second question, wherein the first question is submitted chronologically before the second question, submitting to the subject of a question from the first script can include submitting to the subject the first question; and submitting to the subject an additional question from the first script can include re-submitting the first question to the subject prior to submitting to the subject the second question.

A predetermined time period can be determined to have passed between detecting the triggering event and just prior to submitting to the subject an additional question from the first script; and a starting point in the first script can be returned to, followed by re-submitting to the subject questions from the starting point in the first script.

Determining that a response to the second question from the script is stored in memory can include determining that the second question was previously submitted to the subject within a predetermined time period or that information in a response to the second question had been obtained from a physiological monitoring device monitoring the subject. Determining that a response to the second question from the script is stored in memory can include determining that the second question was previously submitted to the subject within a predetermined time period. Determining that a response to the second question from the script is stored in memory can include determining that information in a response to the second question may have been obtained from a physiological monitoring device monitoring the subject.

Determining whether the subject requires emergency services can include detecting keywords indicative of distress. The keywords indicative of distress can include “Help” or “Emergency”. Determining whether the subject requires emergency services can include generating one or more questions regarding a physical or mental condition of the subject and determining a likelihood of a medical condition from one or more answers by the subject to the one or more questions. The medical condition may be one or more of stroke, heart attack, cardiac arrest, or fall. The medical condition may be a stroke, and generating one or more questions can include generating questions from a stroke interactive session. Data can be received from a monitoring system configured to monitor the subject. Data can be used to detect an indication of a change in health status of the subject. The computer generated verbal interaction can be initiated to detect an indication of a change in health status of the subject. The data can include data concerning a physical condition of the subject. Generating synthesized speech can include selecting speech based on the data. The initiation of a computer generated verbal interaction can include determining in the computer a time to initiate the computer generated verbal interaction, such as following a predetermined schedule. The generation of synthesized speech, receiving a digitized response, performing speech recognition on the digitized response, and determining whether the subject requires emergency services can be performed in a system installed in a residence of the subject or in a mobile system carried by the subject. The generation of synthesized speech, receiving a digitized response, performing speech recognition on the digitized response, and determining whether the subject requires emergency services can be performed without contacting a computer system outside the residence of the subject. Alerting a predetermined contact can comprise generating a telephone call on a plain old telephone service (POTS) telephone line. Alerting a predetermined contact can comprise generating a call over a Wi-Fi network, over a mobile telephone network, or over the Internet. The generation of synthesized speech, receiving a digitized response, performing speech recognition on the digitized response, and determining whether the subject requires emergency services can be performed without contacting a computer system outside the mobile system. Alerting a predetermined contact can comprise generating a telephone call on a cellular telephone.

The techniques and systems described herein may provide one or more of the following advantages. A system for monitoring a person can determine when a person is in need of assistance, such as when the person is in danger or is having physiological problems that could lead to or indicate an SHE. The system can be used with people having compromised health, such as the sick or elderly, or with others who need some low level of supervision, such as a child or a person with minor mental problems. The systems provide early detection of any potential problem. Because when a person is in danger of injury or an SHE, whether the danger is health-related or not, timeliness in addressing the danger can allow the problem to be corrected or problem to be averted. Thus, the systems can prevent serious harm from happening to a person.

The systems may interact with a client in a way that mimics a natural way of speaking. The interaction can make the person being monitored feel more comfortable with the system, which can lead to the system being able to elicit more information from the person than with other systems. Also, the system may be able to start a conversation regarding one topic and switch to another conversation, just as humans do when communicating, thereby focusing on a higher priority need at an appropriate time. When the system determines that emergency services should be called to help the person, the system automatically places the call.

The system may initiate conversations with the subject. Thus, even if a person forgets that they have a tool for contacting emergency services when they are aware of a problem or if they do not have easy access to that tool at the time they need it, the system can automatically contact emergency services. Because the system can actively monitor for problems, the person being monitored does not need to do anything to contact emergency services. Sometimes the person being monitored is not even aware that a problem may be about to occur. The system may be able to detect warning signs that even the person being monitored is not aware of. Because the system may be able to detect a problem very early on, emergency help can be contacted even sooner than they might otherwise be called.

The system may also be able to use conversation-based interaction to minimize incorrect conclusions about the person's status. For example, a physiological monitor may indicate that the person is having a serious heart condition, but a verbal check of the client may indicate that the monitor lead that indicated the condition simply fell off. This may reduce the amount of false alarms generated by standard monitoring devices.

The system may also be used to help people with chronic disease, such as heart disease or diabetes, to carry out disease self-management. For example, the system can remind a person to take his/her medication at the appropriate time and on an ongoing basis. In addition, the system can be used as a platform to develop devices that carry out custom conversation-based applications. A developer of a custom conversation-based application can create custom data, and custom software if required, that is then loaded into the system.

A system that monitors the person can either be carried by the person or sit in the person's home or workspace. The monitoring component includes the scripts that are used to interact with the person being monitored. Therefore, the system is not required to go over the Internet or over a phone line in order to obtain questions to ask the person to carry on a conversation with the person. Therefore, the system can provide a self contained device for monitoring, which does not need to connect with an external source of information in order to determine when a problem is occurring or is about to occur. In some instances, the system may provide an efficient replacement for a nurse or nurse aid. The system, unlike a person, can operate twenty four hours a day.

The systems can help a person who is being monitored in a varied of scenarios. If the person is not aware of an SHE occurring, the person's condition can get progressively worse, at which point the condition could become serious. A monitoring system can detect the problem before it becomes serious. Alternatively, the person may not realize that an early warning sign is associated with a serious condition, such as a heart attack. In this case, the system may detect the warning sign, even when the person does not. A system can help a person who has become physically incapacitated, and cannot move or call for help. The system can also help out when the person is not certain what to do in the event of an emergency. The system can probe for more information when a person notices an issue that may or may not indicate a serious condition or call emergency services when the person calls out for help and would otherwise not be heard. A monitoring system can determine when a person is responding inappropriately, such as with no response or a wrong response, and conclude that the person needs help.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic of a emergency detection and response system.

FIG. 2 is a schematic of a monitoring unit.

FIG. 3 is a schematic of the functional components of a monitoring unit.

FIG. 4 is a flow chart of a verbal interaction with a client.

FIG. 5 is a flow chart of a method of carrying on an interrupted conversation with a client.

FIG. 6 is a flow chart of routinely having verbal interactions with the client.

FIG. 7 is flow chart of a monitoring a client's status over time.

FIG. 8 is a flow chart of determining when emergency services need to be called.

FIG. 9 is a flow chart of determining that the client is experiencing an SHE.

FIG. 10 is a schematic diagram of the data structures and table used by the system.

FIGS. 11A and 11B show a flow diagram of the computer-human verbal interaction process.

Like reference symbols in the various drawings indicate like elements.

### DETAILED DESCRIPTION

A monitoring unit can be used to monitor the health or safety of a subject or person being monitored, also referred to herein as a client. The unit communicates with the client using computer generated verbal questions and accepts verbal responses from the client to determine the client's health or safety status. The monitoring unit can detect that a client may be experiencing, or about to experience, a serious health condition, by verbally interacting with the client. In addition to detecting SHEs, the system can detect early warning signs, such as health symptoms or health-related phenomena, that precede an SHE. In this case, the monitoring unit goes into a probing mode of operation. The unit begins to ask the person a number of questions to help it decide if the situation has a significant probability of being a health emergency. The techniques described herein use the concept of Interaction-Monitored Parameters (IMP). An IMP refers to a specific piece of information that is identifiable by verbal interaction means. An example of an IMP is pain in the center of the subject's chest. An IMP can be assigned a value, such as no, slight, moderate, serious, or severe. A number system could also be used for the values.

The unit can be used in a routine monitoring mode. That is, the unit can regularly check in with the client to determine the client's status and whether someone needs be alerted about the client's status, such as an emergency service. In any situation, the unit can simulate a human interaction with the client to determine the client's status. The unit can determine from the interaction with the client whether the client's responses are responses that would be expected of a client who is in a normal state or if an emergency is occurring. The unit can also determine information from the quality of the client's response whether an emergency is occurring.

The monitoring unit can be a stationary unit or a mobile unit. The stationary unit can sit in a client's home or office. The mobile unit can be carried around with the user. Either unit includes scripts that are designed to elicit information from the client. Because the unit has the scripts built in, the unit need not connect over the Internet or another communication line to obtain questions to use when querying the client.

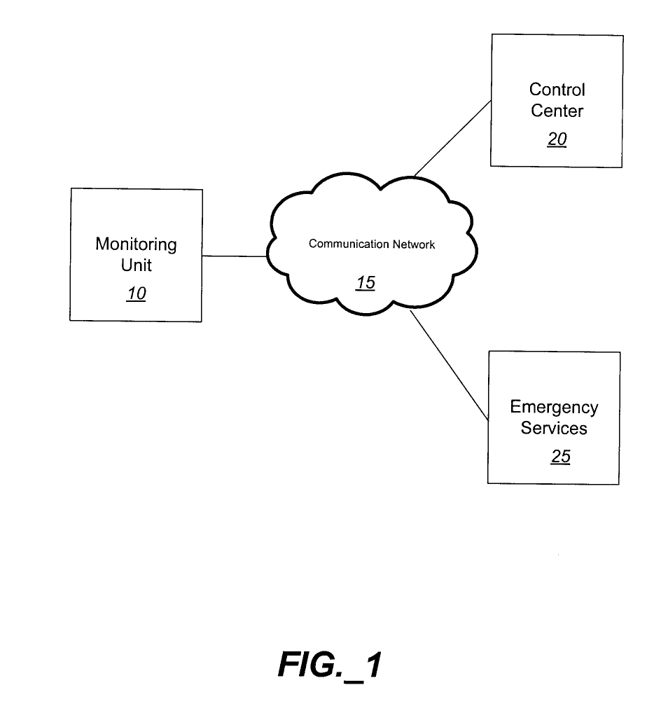

Referring to FIG. 1, a system for monitoring health and detecting emergencies in real time is shown. A monitoring unit 10 is located near a subject, such as a human, who is to be monitored for early warning signs of an SHE or the occurrence of an SHE. The monitoring unit 10 is local to the client and can be a mobile device or a device to be used in one place, such as the home. The monitoring unit 10 is able to transmit to and receive data from a communication network 15. The communication network 15 can include one or more of the Internet, a mobile telephone network or public service telephone network (PSTN) telephone network. Data from the communication network 15 can also be transmitted to or received from a control center 20 and an emergency services center 25.

The control center 20 can include features, such as a client database, a control center computer system and an emergency response desk. In some embodiments, the control center has a telecommunications server that receives calls from the monitoring unit 10, from emergency button devices, and/or telephone calls directly from clients. In some embodiments, the telecommunications server includes an advanced voice/data PBX. In some embodiments, the telecommunications server is connected to the PSTN over several trunk groups, such as in-coming trunks for automatic emergency alert calls, in-coming trunks for manual emergency alert call, in-coming trunks for non-emergency calls, and out-going trunks. The control center may have the client's records on file and may be able to display a record, such as when the possibility of an emergency has been detected. The file can includes information, such as name, address, telephone number, client's medical conditions, emergency alert information, the client's health status, and a list of people to call and actions to take in various situations. The control center 20 can have a network management system that automatically and continuously monitors the operation of the system, such as the components of the control center, the communication links between the control center and the monitoring units 10 and the client's equipment. A high speed local area network capable of carrying both voice and data can connect all of the components at the control center together.

The control center 20 can have emergency response personnel on duty to evaluate a situation. The emergency response personnel can contact the emergency services center 25. Alternatively, the monitoring unit 10 contacts the emergency services center 25 directly. The emergency services center 25 is able to send an emergency response personnel to assist a subject in the event of an SHE.

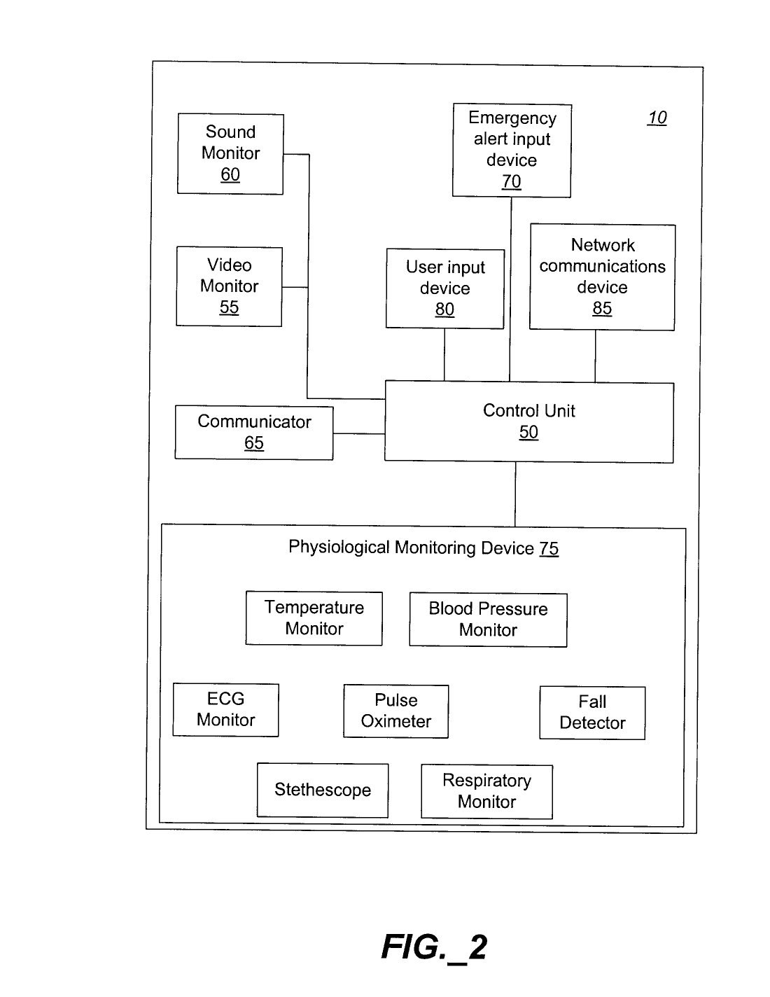

Referring to FIG. 2, in some embodiments, the monitoring unit 10 is a system that includes one or more of the following components, either separately or bundled into one or more units. The monitoring unit 10 includes a control unit 50. The control unit 50 can be a small micro-controller-based device that communicates with the various other monitoring and interaction devices, either over a wired or wireless connection. The control unit 50 analyses data that it receives from the monitors, in some embodiments looking for the early warning signs of health emergencies, or the occurrences of health emergencies. The control unit 50 also carries out various actions, including calling an emergency response service. In some embodiments, the control unit 50 has telecommunications capabilities and can communicate over the regular telephone network or over another type of wired network or over a wireless network. The control unit 50 can also store, upload and download saved parameter data to or from the control center. The control unit can include components, such as a micro-controller board, a power supply and a mass storage unit, such as for saving parameter values and holding applications and data in data tables and memory. The memory can include volatile or non-volatile memory. A micro-controller board can include a microprocessor, memory, one or more I/O ports, a multi-tasking operating system, a clock and various system utilities, including date software utility. The I/O expansion card can provide additional I/O ports to control unit. The card can plug into the backplane of the micro-controller board and can be used in connecting to some of the devices described herein. The mass storage unit can store scripts, table data, and other data, as described further herein.

A communicator 65 can include a built-in microphone that picks up the person's voice, and transmits this signal to the control unit 50. The communicator 65 also has a built-in speaker. The control unit 50 sends computer-generated speech to the communicator 65, which is “spoken” to the person, through this speaker. In some embodiments, the communicator 65 can communicate wirelessly to the control unit 50 using a wireless transceiver. In some embodiments, the communicator 65 is a small device that is worn. In other embodiments, the communicator 65 and the control unit 50 are in a mobile communications device, such as a mobile phone. In some embodiments, the communicator 65 is similar to a telephone with a speakerphone therein.

The communicator 65 in communication with the control unit 50 can also detect ambient noise and sounds from the person and send an analog or digital reproduction of the noise to the control unit 50. The communicator 65, in association with special sound recognition software in the control unit 50, can detect events, such as a glass breaking or a person falling, which can indicate a problem. The control unit 50 can save information about a detected sound in local data store for further analysis. In some embodiments, the control unit 50 uses the concept of sound-monitored parameters, which detects specifically monitored sounds, and associates a value with the sounds, such as no, slight, some or loud.

An emergency alert input device 70 is a small device that can be worn by the client, or person being monitored, such as around the neck or on the wrist. The emergency alert input device 70 consists of a button and a wireless transmitter. The emergency alert input device 70 wirelessly communicates with the control unit 50. When the client feels that they are experiencing a serious health situation, they press the button. This initiates an emergency call to the control center or emergency services. Suitable emergency alert input devices 70 are available from Koninklijke Philips N. V. in Amsterdam, the Netherlands.

In some embodiments, the emergency alert input device 70 has a separate control unit that is in direct communication with the client's telephone system. The emergency alert control unit can automatically call the emergency service when the client activates the emergency alert input device 70, bypassing the control unit 50 all together.

One or more physiological monitoring devices 75 can send continuously or periodically detect and monitor various physiological parameters of the person, and then wirelessly transmit this data to the control unit 50, in real time. Suitable monitoring devices can include an ECG monitor, pulse oximeter, blood pressure meter, fall detector, blood glucose monitor, digital stethoscope and thermometer. The physiological monitoring devices 75 can transmit their signals to the control unit 50, which can then save the data, or values, in local data storage. The control unit can process the signal to extract physiological values and then saves the values in local storage. The system can include none, one, two, three, four, five, six, seven, eight or more physiological monitoring devices.

An ECG monitor is a small electronic unit with three wires that come out of it, and in some instances has five or more wires. These wires are attached to electrodes. The electrodes are affixed to a person's skin in the chest area, and they make electrical contact with the skin. The ECG monitor records a person's ECG signal (electrical heart signal) on a continuous basis. The signal is usually sampled at 200-500 samples per second, converted into 12-bit or 16-bit data, and sent to the control unit. The ECG monitor can be battery powered. The ECG monitor can also wirelessly receive data or instructions from the control unit, over the wireless link. This includes an instruction to test whether the electrodes are properly affixed to the person's skin. In addition, the ECG monitor can measure more than one ECG signal. Suitable ECG monitors are available from CardioNet, located in San Diego, Calif., and Recom Managed Systems, located in Valley Village, Calif.

A pulse oximeter is a small device that normally clips on the client's finger or ear lobe or is worn like a ring on one's finger. The purpose of the pulse oximeter is to measure the blood oxygen saturation value of the client. Blood oxygen saturation refers to the percentage of hemoglobin in the blood that is carrying oxygen; an average rating is 95%.

A wireless (ambulatory) blood pressure monitor consists of an inflatable cuff that normally is worn around the upper arm, a small air pump, a small electronic control unit, and a transmitter. To measure the client's blood pressure, the air pump first inflates the cuff. Then the air in the cuff is slowly let out. The monitor then transmits the reading to the control unit. The amount of data is very small and can be left on all the time. The monitor can be auto-controlled by the control unit. Alternatively, the monitor could be manually operated by the client. The client may only put it on when he/she is taking a measurement.

A fall detection monitor is a small electronic unit that clipped onto the person, usually on the belt. The unit contains two, or more, accelerometers that measures the acceleration of the unit on a continuous basis. In particular, the fall detection monitor detects when the person falls hard to the floor. Suitable fall detection monitors are available from Health Watch, located in Boca Raton, Fla.

A user input device 80 can allow a client to interact/communicate with the control unit 50, such as through a screen, buttons and/or keypad, similar to the personal digital assistant or communications device. Text can be send to a screen on the device, which a client can read. The screen can be small, such as 2″×2″ in size and can be a color or black and white screen. If the text to be presented on the screen is more than can fit on one screen, the user input device 80 can allow the client to scroll through the text. The device can have about 16 keys, or more, such as in an alphanumeric keyboard. Ideally, the user input device 80 has keys that are sufficiently large for an elderly person or someone with limited mobility, dexterity or eyesight to be able to use. The client can use the user input device 80 to manually enter information, such as numbers from a monitoring device. The user input device 80 can also be used when a client is hard of hearing or has difficulty understanding, when the client prefers to use the input device 80 over speaking to the unit, such as when the client is in public, e.g., in a shopping mall, at work on the bus, or when excessive noise interferes with the operation of the communicator 65. In some embodiments, the user input device 80 is able to ring, vibrate or light up to get the client's attention.

A network communications device 85 can include one or more of various devices that enable communications between the control unit 50 and the control center, emergency services or some other location. Exemplary devices can include a landline telephone, a mobile telephone, a modem, such as a voice/data modem or the MultiModemDSVD from MultiTech Systems in Mounds View, Minn., a telephone line, an Internet connection, a Wi-Fi network, a cellular network or other suitable device for communicating with the communications network. In some embodiments, the mobile phone includes a GPS locator unit. The locator unit allows the mobile telephone to communicate the client's location in the event that emergency services need to be called and they need to find the client.

One or more of the devices described herein can be worn by the client, such as during the client's normal activities or during sleep. Some of the devices, such as the physiological monitoring devices 75, can be wireless and be worn regularly by the client. Wireless devices allow the client to move freely about. Some of the devices can be made for wearing by the client 24 hours a day, seven days a week. For example, sensors can be embedded in the client's clothing or in special garments worn by the client. The wireless receivers or wireless transceivers used can have an operating distance of 5 feet, 10 feet or more, such as 200 feet or more, and can work through walls, and have a data rate necessary to support the associated monitoring device. Suitable wireless devices can be based on technologies, such as Bluetooth, ZigBee and Ultra Wideband. In some embodiments, the wireless monitors are implanted in the client.

Because one or more of the devices may be battery operated, a charging device can be included for charging batteries. In a mobile version of the system described herein, a cradle is provided for charging a mobile portion of the control unit and can enable communications between the mobile portion of the control unit and a base unit of the control unit. In some embodiments, a mobile version of the control unit 50 is worn or carried by the client, such as when the client leaves the house. When the client places the mobile portion of the control unit 50 in the cradle, the mobile portion can analyze the data it receives from the client's on-person monitoring devices as well as data that the base receives from other monitoring devices, such as off-person monitoring devices. Off-loading information from the mobile device can free up storage space. Alternatively, the base station can perform the analysis. The data from the mobile portion can also be downloaded into the base.

The control unit can include a back up power supply, such as a battery, for use when the primary power supply has gone down. The control unit may also be able to use the power over a phone line.

One or more of the units described above, such as the control unit, the network communications device and the user input device can be integrated into a single device. Of course, other devices can be optionally included in the integrated device.

In one embodiment, a mobile system that includes the control unit 50 and one or more of the aforementioned components is a mobile telephone. The mobile telephone can have a peripheral-card that transforms the mobile telephone into a suitable control unit 50 or monitoring system. The mobile telephone has data capabilities including a data channel and a data port and the ability to run custom software. In particular, the mobile telephone can activate the telephone to make out-going data calls and handle in-coming data calls and connect the data calls. The mobile phone can also send the client's GPS coordinates to emergency services.

Either the stationary device or the mobile device can be in wired or wireless communication with the communicator. The client can wear the communicator, such as a lavaliere pinned or clipped to the client's clothing or worn suspended from the client's neck. With the mobile device, the client need not speak into the mobile phone, but can use the communicator, instead.

In some embodiments, the control unit is a self contained device that includes the controller, memory, power supply, speech recognition software, speech synthesis software and software that enables the unit to contact emergency services. In one embodiment, the self contained device also includes a speaker and a microphone for communicating with the client. As noted herein, in embodiments, the mass storage unit the scripts and other data used to communicate with the client and components that enable the control unit to determine when the emergency services should be called without connecting to an external system to query script for conducting a conversation with the client.

Any device used as a control unit, whether it is a mobile or stationary control unit (for mobile or home use), a mobile telephone or other device, can include drivers, software and hardware that allow the control unit to communicate with the devices that are in communication with the device.

Optionally, the system can have a video monitor 55 in communication with the control unit 50. The video monitor 55 and control unit 50 can capture video images of the person as she/he moves about. These images are sent to the control unit 50 for analysis, such as to detect indications of possible problems with the client. The video monitor 55 can function to look for specific, significant video occurrences and can save the information in local data storage for further analysis. The video monitor can capture images of the client swaying, falling, waving arms or legs, or performing tests, such as the client's ability to control his or her arms. In some embodiments, the video monitor has associated with it a video-monitored parameters for the events it captures, such as no, slight, some or significant.

Other optional monitors include a pressure-sensitive mat, such as a mat placed under the client's mattress, which can sense when the client is in bed and motion detectors.

In some embodiments, the system primarily includes the verbal interaction capabilities. In some embodiments, the system includes the verbal interaction capabilities in addition to one or more of the physiological parameters monitoring devices. In some embodiments, the system includes the verbal interaction capabilities, one or more of the physiological parameters monitoring devices, and sound/image recognition capabilities. In some embodiments, the system includes the verbal interaction capabilities, one or more of the physiological parameters monitoring devices, a sound/image recognition device and a user input capabilities.

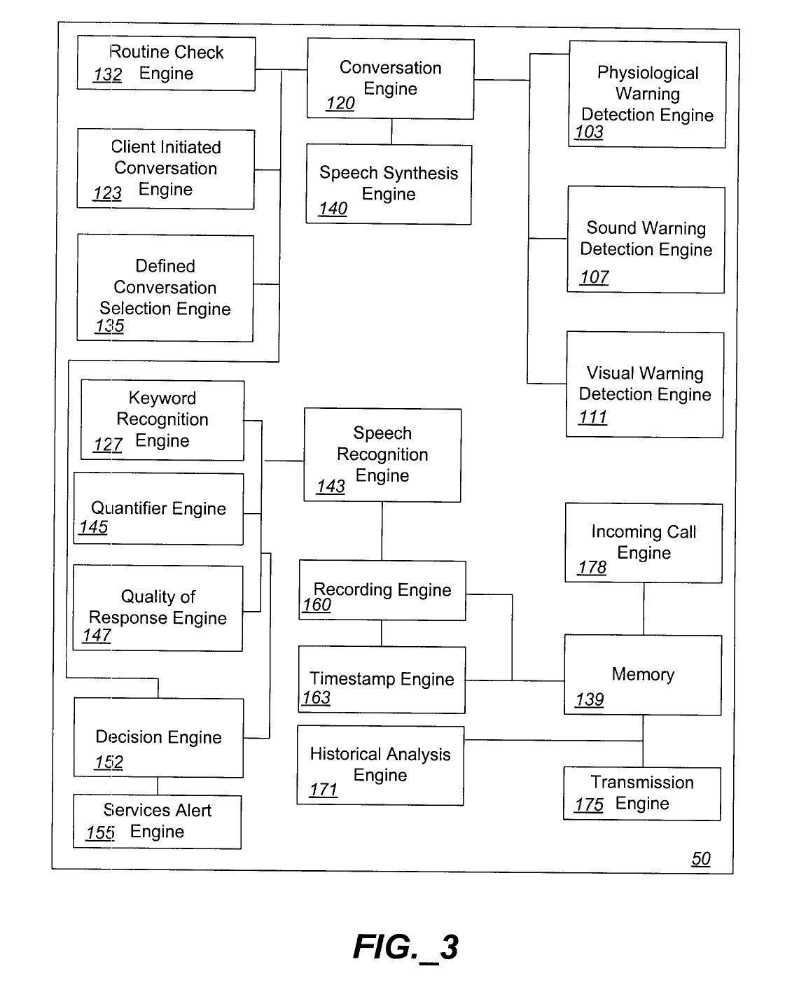

Referring to FIG. 3, the control unit 50 can include one or more of the following engines. Each of the engines described herein runs routines suitable to perform the job of the engine. Some of the engines receive and analyze data from the components in communication with the control unit 50, including a physiological warning detection engine 103, a sound warning detection engine 107 and a visual warning detection engine 111. When one or more of these engines detects an occurrence of an event that may indicate an emergency, a conversation engine 120 is initiated. The conversation engine 120 provides computer-human verbal interaction (CHVI) with the client.

CHVI refers to a computer-based device obtaining information from a person, by verbal means, simulating a conversation with the person in such a way that the conversation seems to be a natural conversation that the client would have with another human. CHVI is used to verbally obtain specific information from an individual that is relevant to the current emergency detection activity and that often cannot be obtained any other way. The information is used to decide, or help decide, whether the situation is an emergency or not, i.e., that the probability is high enough to justify alerting emergency service.

In addition to the physiological warning detection engine 103, a sound warning detection engine 107 or a visual warning detection engine 111 initiating the conversation engine 120, a client initiated conversation engine 123 can prompt the conversation engine 120 to check the client's status. The client initiated conversation engine 123 detects when a client says something without already being involved in a conversation with the control unit 50. In some embodiments, the control unit 50 has a keyword engine 127 to detect when the client says a keyword, such as “help”, “ouch”, “emergency”, or other predetermined word that indicates that the client would like assistance. The keyword engine 127 then directs the conversation engine 120 to interact with the client. A routine check engine 132 can periodically prompt the conversation engine 120 to check in with the client or probe the client for current status information. The routine check engine 132 can be prompted to check the client on a schedule, at predetermined time periods, if the client has not spoken for a predetermined time or randomly.

Once the conversation engine 120 is initiated, the defined conversation selection engine 135 selects an appropriate conversation to have with the client. For example, if the client has called for help, the defined conversation selection engine 135 may select a script that asks the client to describe what has happened or what type of help is required. Alternatively, if it is time for a routine check on the client, the defined conversation selection engine 135 selects a script that checks in on the client, asks how he or she is feeling and reminds him or her to take their medication. Many scripts can be programmed and stored in memory 139 in the control unit 50 for the defined conversation selection engine 135 to select from. Once the script has been selected, a speech synthesis engine 140 forms verbal speech from the script and sends the speech to a speaker associated with the control unit 50 or to a speaker in a wireless communicator.

Responses from the client are translated by a speech recognition engine 143, which converts the audio signal into text. A quantifier engine 145 assigns a value to some responses. For example, if the client has pain, the quantifier engine 145 can assign different values to none, some, moderate, and severe pain. A response quality engine 147 determines the quality of the response, which is different from the actual response provided by the client. The response quality engine 147 can determine if the response was an expected response or not an expected response, if the client did not reply to a question within a reasonable period of time, whether the reply contained one or more words that are not recognized, that the reply was a reply that is not anticipated or that the reply is garbled and therefore unparseable. In some embodiments, the response quality engine 147 also recognizes voice inflection and can determine if a client's voice has characteristics, such as fear, anger or emotional distress. A decision engine 152 uses the text and/or the quality of the response to decide what action to take next. The decision engine 152 can decide what action to carry out next, including what question to ask next, whether to repeat a question, skip a question in the script, switch to a different script or conversation, decide that there is a problem or decide to contact an emergency service. When a different script is to be selected, the decision engine 152 can determine the priority between continuing with one script or conversation versus switching to a new conversation. If the decision engine 152 decides to contact emergency services, the services alert engine 155 is initiated.

The services alert engine 155 can send information, such as the client's location, an emergency summary report and real time parameter values based on the client's status, to emergency services. The services alert engine 155 can establish a connection with a service provider, such as an emergency service provider. Additionally, the services alert engine 155 can work with the client to help with equipment set-up. When the system stops working properly or when equipment is not connected properly, the services alert engine 155 can establish a call to a service provider that is then able to help the client get the equipment operating again. In some embodiments, the services alert engine 155 transfers input from the client to the service provider.

The responses from the client, including the quality, the text and a value, can be recorded and stored to memory by a recording engine 160. A timestamp engine 163 can timestamp the response prior or subsequent to storage. A historical analysis engine 171 can review previous responses to determine trends, which can be used to set a baseline for the client's responses. In some embodiments, only select responses are saved to memory, such as responses that indicate that a non-normal event is occurring, such as a fall, pain, numbness or other such medical or dangerous event.

Any of the data collected can be saved to memory 139 to send to a central database, such as at the control center 20, by a transmission engine 175. The transmission engine 175 can transmit data automatically, on a scheduled basis, or as directed. If data is transmitted on a scheduled basis, the schedule can be varied. Either all values or only a summary of the values may be transmitted. Once the data has been transmitted, the data can be analyzed for long term health monitoring. The client's health care provider can also access the data to supplement information received during an examination to review in preparation for an examination or other medical procedure or to discover long term health trends. Long term health trends can be used to develop an effective health care plan for the client or to monitor the long term effect of a new medical treatment on the individual.

An incoming call engine 178 can allow the control unit 50 to handle incoming calls, establish caller-to-communicator connections, access client parameter data and perform a check-up or polling call. The incoming call engine 178 may be used when the control center is unable to reach the client by telephone. The incoming call engine 178 can allow for text can be received by the control unit 50 and converted to speech, such as by the speech synthesis engine 140, to be communicated to the client, or sent to the client's user input device. If a request for data is made, the incoming call engine 178 can handle the request and initiate the transmission engine 175. Regarding the polling call, the engine can be provided with one of two codes on a recurring basis, an “emergency detected” code or a “no emergency” code. If an incoming polling call is received, the incoming call engine 178 can pass on the latest code that it has received. Polling calls can be received periodically, such as once every 10 to 20 seconds. The polling call can function as a backup emergency alert system. The incoming call engine 178 can also be used when a remote system wants to update the memory, such as by changing or adding new scripts.

To add a new device to the control unit, a suitable device driver, data handling and processing modules can be added and new parameters associated with the device can be added to tables as required.

As noted, a device can either be a stationary type device, such as one that is used in a client's home, or a mobile device. In either type of device, the components can be similar. In a mobile device, however, the functionality may be decreased in favor of control unit size or battery power conservation. Conversely, some functionality is increased in the mobile device. For example, the sound environment in the home is different from outside the home. Outside the home, the sound environment can be more complex, because of traffic, other people, or other ambient noise. Therefore, the sound engine in the mobile device can be more sophisticated to differentiate sounds that are relevant to the client's health versus those that are not. In particular, a glass breaking in the home may indicate that the client is experiencing an emergency when the same may not be true outside the home. The mobile unit may also have GPS software to allow the client to be located outside the home. The mobile device can also have an emergency button and corresponding emergency software. The OS for the mobile device, or the user input device, can be one designed for a small device, such as Tiny-OS.

The system can carry out verbal interaction using interaction sessions and interaction units. An interaction unit is one round of interaction between the system and the client. For example, an interaction unit can contain data that enables the device to obtain information from a person related to their current general health status. An interaction unit involves the device communicating something to the client, and then the client communicating something back to the device, and the device determining what to do next, based on the client's reply. Therefore, the interactive session can include a number of interactive units. Each interaction session has a specific objective, for example, to determine whether the client is having early warning signs of a stroke or whether the client is having early warning signs of a heart attack. An interaction session consists of all the data required for the system to carry out one conversation with a client. Different interactive sessions can be used with the client, such as throughout the day. Probing interactive sessions attempt to determine whether the client is in a potentially serious condition. For example, the control unit may observe that the client's heart has suddenly skipped a few beats. The control unit can use a probing interactive session to ask the client a few questions related to early warning signs of a heart attack. A routine interactive session is an interactive session that is generally not involved in a situation that is serious or may be serious and is used to routinely communicate with the client.

The system can extract different types of information from the client's responses. The first type of information is the words the client uses to respond to a question posed by the system. The words can indicate an actual answer provided by the client, such as “yes”, “no”, “a little”, or “in my arm”. The system can determine from the response whether it is an expected response or whether the system needs more information to make a decision, such as when the answer is an unexpected answer or the answer is outside of the system's known vocabulary. In addition, the system can determine the quality of the response. For example, the client may delay in providing a response. The client may provide a garbled response, which cannot be understood by the system. Any of these conditions can indicate that the client is experiencing a health condition or crisis that requires emergency care or further investigation to determine the client's health status.

Any of the devices, such as the monitoring devices, and components can be used to determine when a trigger event occurs. For example, a physiological monitor can determine a trigger event, such as high blood pressure. The trigger event can be a value that is outside of a predetermined range, such as higher than a predetermine high level, or lower than a predetermined low level. When the system receives notice of the trigger event, the system uses the trigger event to perform one or more of the following three tasks. The system may decide based on the trigger event to probe the client for more information. Alternatively, the system may automatically call emergency services. If the system probes the client for more information, the system can use the trigger event to determine an appropriate conversation for having with the client. For example, if the client's blood pressure has risen, the system may begin a conversation that asks the client how he feels or a conversation that asks whether the client has taken his blood pressure medication that day. The system can also use the trigger event as a weighting factor to determine whether to call for help. For example, if the blood pressure is moderately high, the system may decide to check back with the client later, such as five minutes later, to see how he is doing. If the blood pressure is very high, the system may be more likely to contact emergency services.

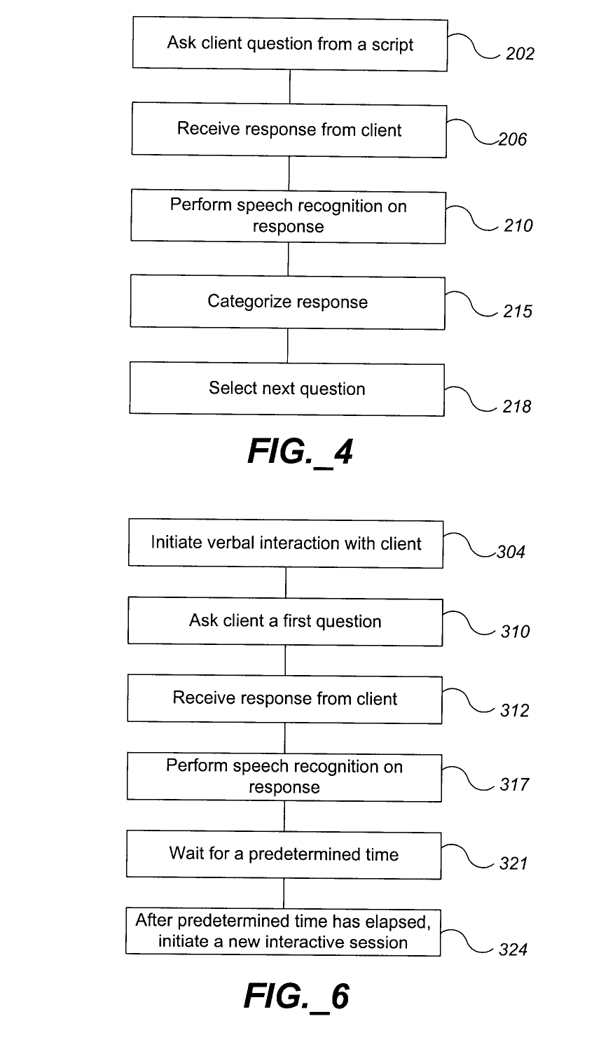

Referring to FIG. 4, a conversation-based verbal interaction used by the system to either probe the client for information or that is part of a routine check is described. In some conversations, such as the routine check, the system initiates a conversation with the client, such as by saying, “Good morning John”. The system then asks the client a question from a script (step 202). The question can be a question, such as “Have you taken your blood pressure today?” or “Do you have pain?” The client then responds. The system receives the client's response (step 206). The system performs speech recognition on the response to translate the speech to text (step 210). The text is then categorized (step 215). The system decides what to say to the client next, based on the category of the response. For example, if the client response “Yes” to the question, “Do you have pain?”, the system can ask, “Where does it hurt?”. However, if the client responds “No” to the same question, the system may respond, “That's good. I'll check in with you tomorrow.” The system's response is selected from the next appropriate question, such as by selecting the next question in a script, or according to the response received from the client (step 218).

The system can use responses stored in memory to determine the next question to pose to the client. For example, the system may have recently asked a question and therefore knows the answer to a question in the script. In this case, the system can skip that question if it comes up as a question in a script. Alternatively, the system knows to that it can skip a question because it has received information from a physiological monitoring device. The system can timestamp responses received by the client to help the system determine how old the response is. If the response is fairly recent, such as less than a minute or two minutes old, the system may decide to skip asking the question again.

As noted, a client can either initiate a conversation or respond in such a way that initiates a new conversation. For example, the system may ask, “Did you take your pills today?”, and the client responds, “Oh, I just felt a sharp pain in my chest.” In this situation, the system can recognize when the client is initiating a new conversation, as opposed to partaking in an existing conversation and the system knows switch the conversation to respond to the client's response.

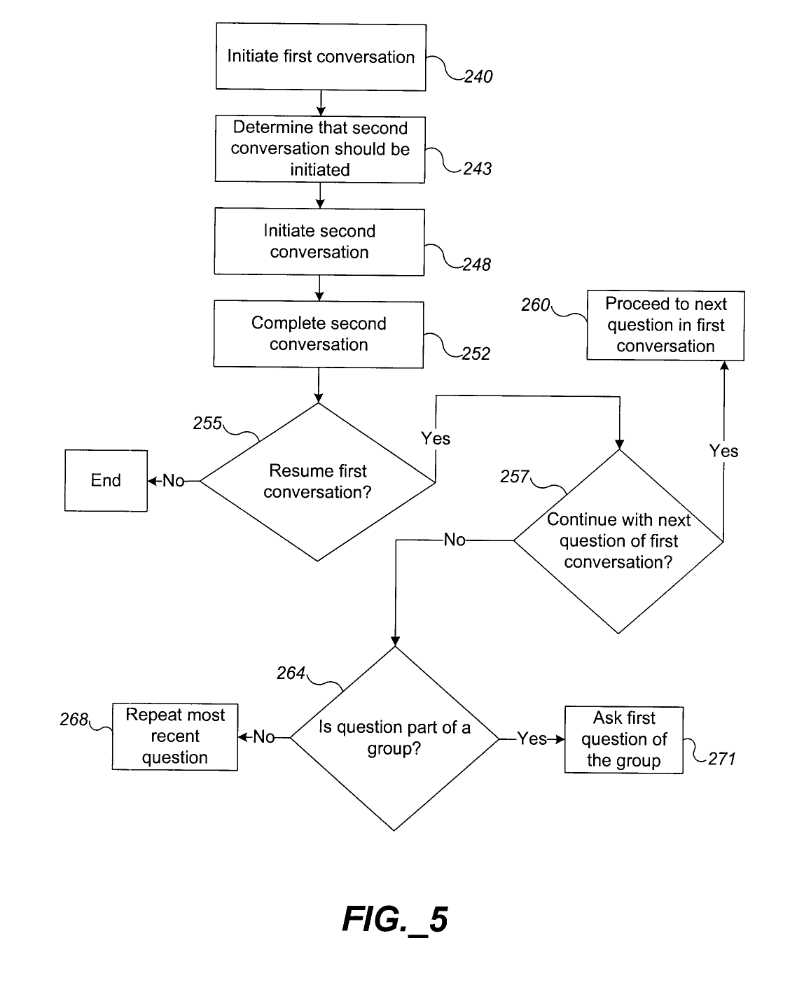

The system can switch from a script that is being used to ask questions of the client to begin asking questions from another script to change a conversation. For example, the system can be asking the client questions from a general script. If the system detects that another script would be more helpful to elicit particular responses from the client or to detect a possible emergency, the system can stop mid-conversation and switch to the other script, as further described in FIG. 5. The system initiates the first conversation (step 240). After asking at least one question from the script, a trigger event occurs that causes the system to determine that a second conversation should be initiated, interrupting the first conversation (step 243). The event can be the answer to a question from the first conversation, a sound in the background, a signal from a physiological monitor, the quality of a response from the client or other such trigger. In some cases, the event indicates that the client may be experiencing or be about to experience an SHE or a serious health condition. In some embodiments, different conversations or scripts are assigned different priority levels and the system decides to move to a different conversation if that conversation has a higher priority level than the first conversation.

The system triggers a second conversation (step 248). The system completes the second conversation (step 252). At the end of the second conversation, the system then decides whether to go back to the first conversation (step 255). In some instances, the system will decide that the first conversation is not necessary to complete and will end the session.

If the system decides to go back to the first conversation, the system then determines whether to pick up where it left off in the first conversation and continue with the next question of the first conversation (step 257). If proceeding to the next question in the first conversation would not be confusing to the client, the system can proceed to the next question (step 260). If there has been too long of a lapse since the first conversation was interrupted or if the next question in the group of questions would not make sense to the client without the context of the conversation, that is, if the system exceeds a maximum interruption time, the system will not move on to the next question in the conversation. If the system needs to back up at least one question to provide a reminder or context, the system determines whether the most recently asked question is part of a group of questions (step 264). If the question is not part of a group of questions, the system goes back one question and repeats the most recently asked question from the first conversation (step 268). However, if the question is one of a group of questions, the system backs up to the first question of the group and asks the first question of the group (step 271). When the scripts are prepared to form a conversation, groups of related questions are indicated as such.

A group of questions that can be chronologically asked in a conversation may be: “Did you just cough up some phlegm?” “If yes, what color is it?” “Has this been going on all day?” If the client were asked the first or first and second questions and was not asked the following question immediately thereafter, the client may be confused when later asked the subsequent question or may provide an answer within the context of another conversation, that answer not being the answer to a question that the system believes is being posed to the client.

Each time the client speaks, the system can determine whether the client is replying to a statement made by the apparatus, or whether the client is expressing something independent of the present conversation. If the client is expressing a new idea, the system will determine from the words the client is using whether a different conversation should be initiated, thereby interrupting the present conversation.

Of course, more than one conversation can be interrupted, depending on the events that are detected by the system. The system can simultaneously track multiple conversations that are interrupted in this case.

Verbal interaction is an easy, convenient way for a person to be monitored over a long period. One concern, though, is that too much, or too frequent, interaction may annoy the person, or it may cause too much disruption in what the person is doing. When this happens, the person may become less cooperative, and the effectiveness of verbal interaction can decrease.

Every interaction is associated with a trigger condition. A trigger condition specifies when an interaction is to be carried out. By carefully defining these trigger conditions, the system can optimize the frequency of occurrence of these interactions. In this way, there will not be too much interaction, and there will not be too little interaction.

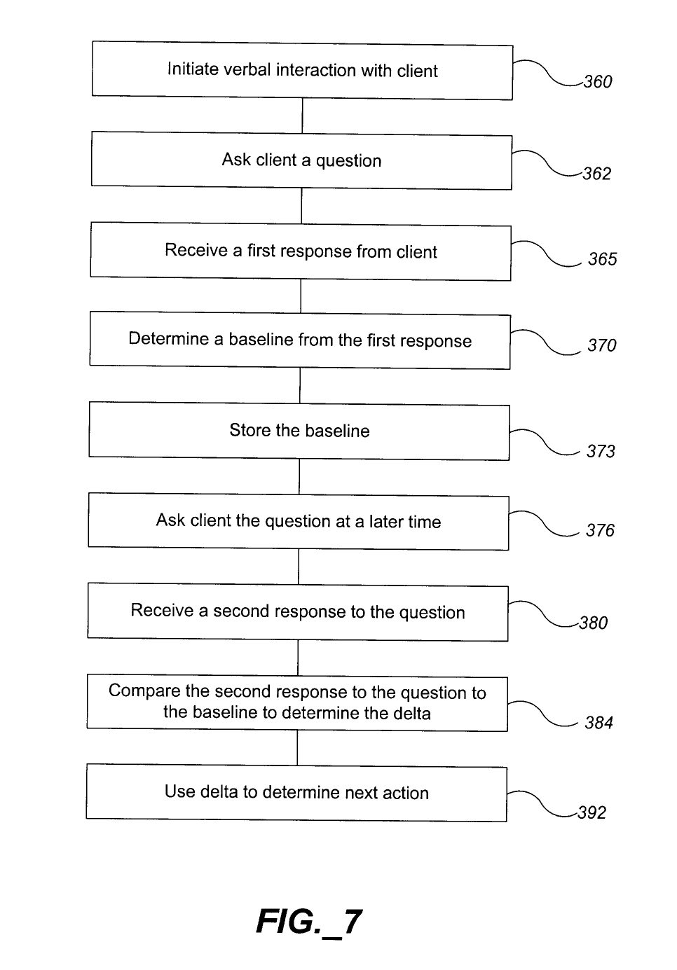

Referring to FIG. 6, the trigger condition can be a time and thus, as noted herein a routine check of the client can occur at predetermined time periods. The system initiates a verbal interaction with the client (step 304). This begins an interactive session with the client. The system asks the client a first question (step 310). The system receives the response from the client (step 312). The system performs speech recognition on the response (step 317). Any subsequent questions or actions are then performed. The system waits for a predetermined time (step 321). After the predetermined time has elapsed, the system initiates a new interactive session with the client (step 324).

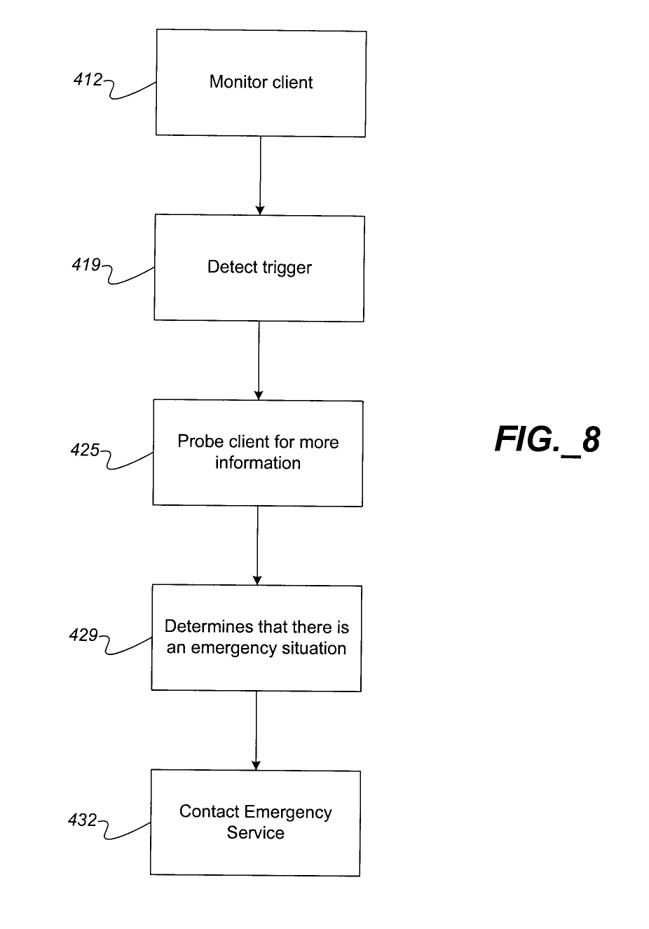

Because the system is able to ask the client questions repeatedly over time, a baseline for the client's response can be set to compare current client status with former status. The baseline can be used for disease management or to indicate that the client's health status has worsened and requires attention. Referring to FIG. 7, the system initiates verbal interaction with the client (step 360). The system asks the client a question (step 362). A first response is received from the client (step 365). A baseline is determined from the first response (step 370). Subsequent responses to the same question can also be received from the client and be used together to determine the baseline or to modify the baseline after it is determined. The baseline is stored (step 373). The client is asked the same, or a similar question, at a later time (step 376). The system receives a second, or subsequent, response from the client (step 380). The second response is compared to the baseline to determine a delta (step 384). Exemplary comparisons can be the amount of delay in receiving a client's response, an amount of pain experienced by a client and whether the client is able to perform certain tasks in a particular way or within a time period. The delta is used to determine the next action taken by the system (step 392). For example, the system may determine that the delta is above a predetermined threshold, thereby indicating that the client's status has changed over time or that the client has experienced a change that requires some attention.

Thus, the system can ask the client questions at spaced intervals to determine the client's progress, that is, if the client is improving or worsening and if help should be called. The system can also record a client's physiological parameters, sound data or image data for later analysis and to be used in combination with later obtained data. For example, if a valid response from the client indicates that the client is having a problem, such as pain, and the client's latest heartrate recorded is greater than a predetermined baseline, such as 125 b/m, and there is an image of him falling within the last 10 mintues, the system can use the text of the client's response and the client's physical or physiological data to determine that help is required and should be called. Similarly, if the client exhibited a physical condition recently and currently that both indicate that the client needs help, such as an abnormally low blood pressure and video images of the client show the client walking unstably, a determination can be made that the client requires emergency services.

In addition to monitoring a client's status, the system can detect the warning signs of an SHE to help prevent the occurrence of SHEs, and to reduce the impact of SHEs if they do occur. The system continuously monitors an individual for early warning signs, and occurrences, of SHEs. When an SHE is detected, the system can auto-alert emergency response services, as described further herein. Therefore, the system can assist the client when the client is not aware of the early warning signs of a potential, imminent health emergency, when the client is aware of the emergency but is unable to call for help or when the client is in an emergency situation, but is not aware of the emergency and is thus unable to do anything about the situation.

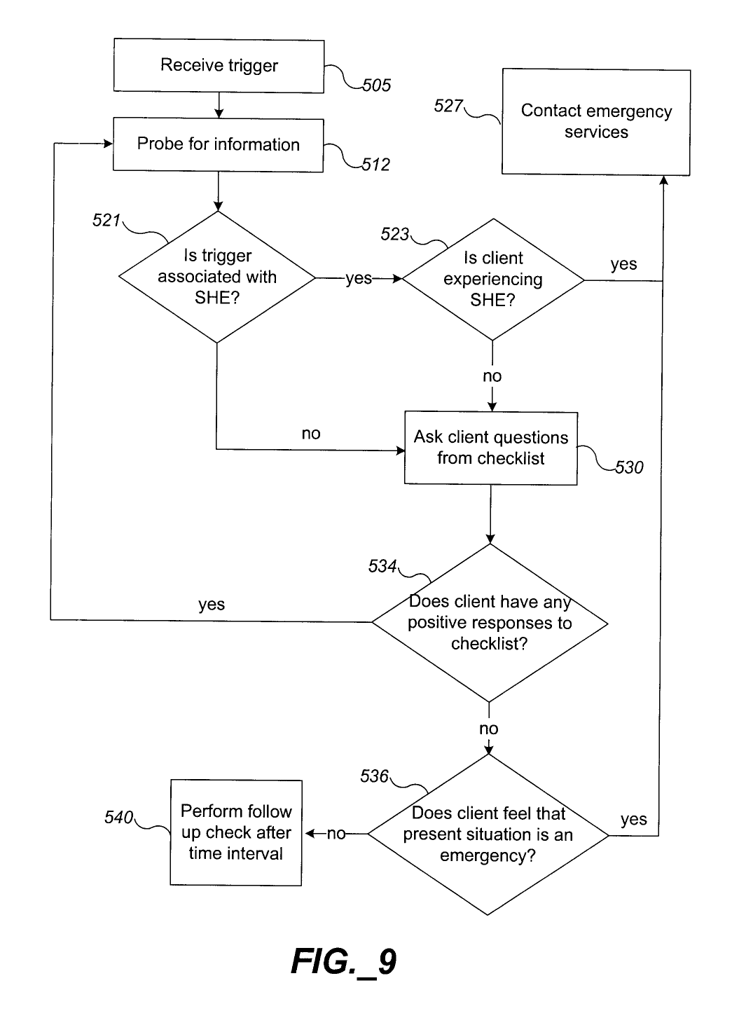

Referring to FIG. 8, to determine and assist the client in the event of an emergency, the system performs the following functions. The system monitors the client generally, such as by monitoring the client's health, safety and/or wellbeing (step 412). The health monitoring can include monitoring physiological parameters, verbal interaction monitored parameters, sound monitored parameters and video monitored parameters. The parameters are obtained and monitored continuously and in real time. For example, the system can routinely have verbal interaction sessions with the client. The routine verbal interaction session carries out a quick, general health check-up on the client.

A trigger is detected (step 419). The trigger could be any of a signal from one of the physiological monitors, a signal from a user input device or emergency alert device, a signal from an alarm component in the client's home, a signal from a video or sound monitor or a signal detecting the client requesting help. The system begins to probe the client to get more information and determine whether there is an actual emergency situation or whether it is a false alarm (step 425). Based on a number of factors, including responses or lack of responsiveness from the client and/or external indications, the system determines that there is an emergency situation occurring (step 429). Exemplary emergencies include stroke, heart attack, cardiac arrest, unconsciousness, loss of responsiveness, loss of understanding, incoherency, a bad fall, severe breathing problems, severe pain, illness, weakness, inability to move or walk, or any other situation where an individual feels that they are experiencing an emergency. Emergency services are contacted (step 432). In some embodiments, the client can call out a key word or phrase, such as “emergency now” that bypasses the probing step and immediately calls the emergency service.

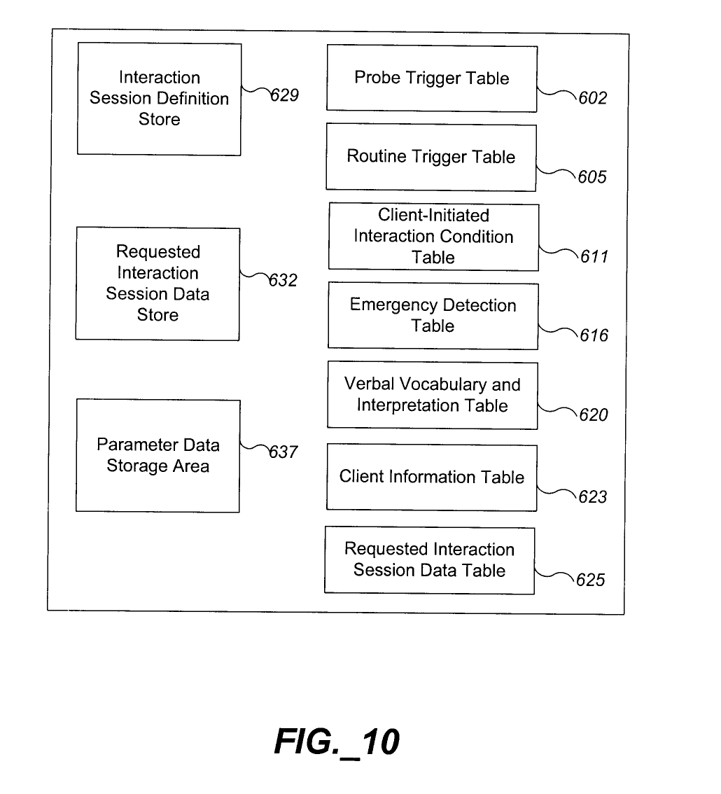

Referring to FIG. 9, in one embodiment, the system determines whether the client is experiencing an SHE or other emergency using the following method. The system received a trigger (step 505). After receiving the trigger, the system begins to probe the client for information (step 512). From the information received from the client, the system determines whether the trigger is associated with an SHE (step 521). If the trigger is associated with an SHE, the system attempts to determine whether the client is actually experiencing an SHE (step 523). This may require further questions or analysis of signals received by the system. If the client is experiencing an SHE, the system contacts emergency services (step 527). The system can provide information associated with the emergency situation when contacting emergency services. Alternatively, or in parallel, the system determines which SHE the client is likely experiencing. If the trigger is not associated with an SHE, or if the client is not actually experiencing an SHE, the system asks the client questions from a checklist (step 530). The checklist can be any list, such as a health watch list or other list that would find indications of a problem. If the client has any positive responses (step 534) to an entry on the checklist, the system can return to the probing step (step 512) to determine what is going on. In returning to the probe step, the system can ask additional or different questions than the first time the client was probed. If the client has no positive responses to the checklist, the client can be asked whether he or she feels as though the present situation is an emergency (step 536). If the client responses positively, the system contacts emergency services (step 527). If the client responses that he or she does not feel that the present situation is an emergency, the system performs a follow up check after some time interval (step 540).

Regardless of whether the system is actively asking the client a routine question or a probing question or is not verbally interacting with the client, the system can be continuously monitoring the client and waiting for a trigger. That is, regardless of what the system is doing in terms of the verbal interaction, in the background the system can be in a trigger detection mode. The system can be constantly listening for a keyword, receiving physiological parameters and checking the parameters for whether they indicate a trigger event has occurred, listening for specified ambient sounds or receiving and processing images of the client to determine if a trigger event has occurred.

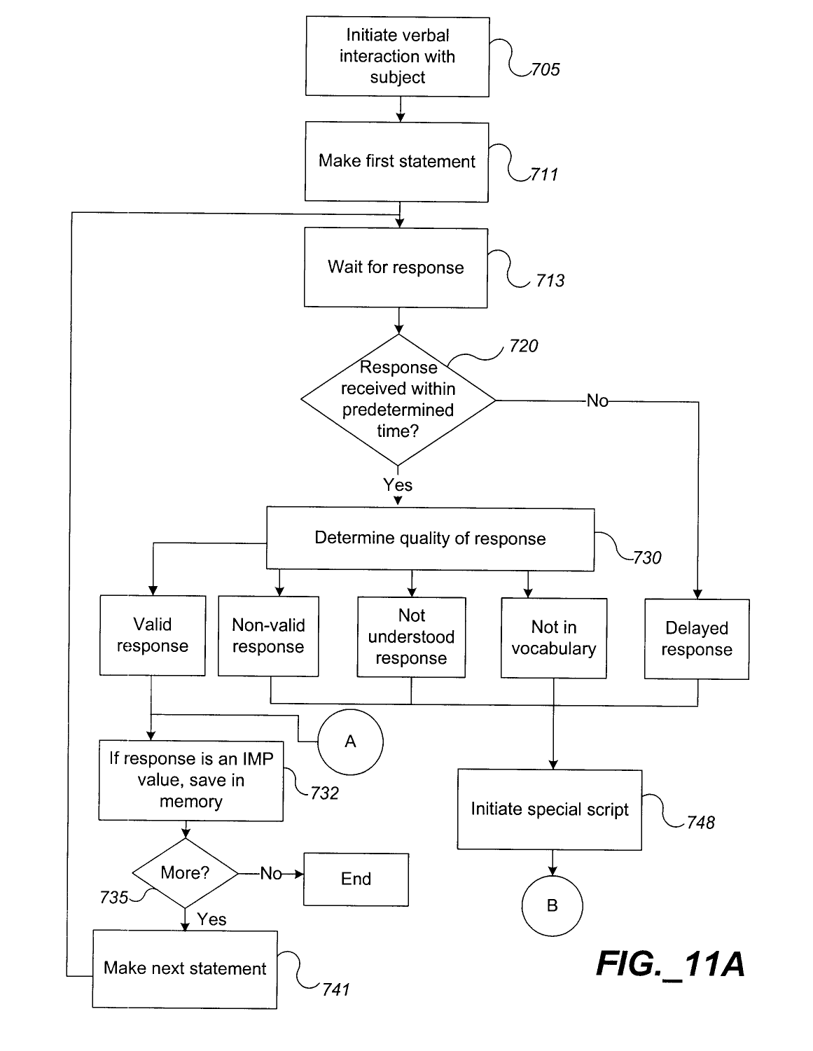

Embodiments of the system can include software as described herein. Referring to FIG. 10, data used by the system can be in data structures, data tables and data stores. The data structures can be the interaction units, the interaction sessions and interaction session definitions (ISD), including output text string (OTS) instructions, conditions—decision statement, and action instructions—decision statement. The data stores can include a parameter data storage area 637 (DSA), a requested interaction (ReIS) session data store 632 and an interaction session definition store 629. The data tables can include a probe trigger table 602, a routine trigger table 605, an emergency detection table 616, a client initiated interaction table 611, a verbal vocabulary and interpretation table 620, a client information table 623 and a requested interaction session data table 625.

The computer based verbal communication can be supported by a virtual human verbal interaction (VHVI) platform. By platform, it is meant that the system consists of all the core elements/components required by a stand alone device to carry out advanced VHVI functionality. The platform can have hardware and software components. Custom data can be added to tailor the system to a user or to an application. Custom software may also be required.

A VHVI-capable device (or VHVI device for short) is a device that carries out an application that involves VHVI. A VHVI device contains technology that enables it to verbally interact with a person in a natural way, that is, the device models the human thinking process associated with verbal interaction.

A VHVI device, that carries out an application can include a microcontroller with a wireless transceiver, a communicator with a wireless transceiver, a VHVI software sub-system, application data for VHVI tables and additional custom application software. The device can perform basic verbal interaction, recognize and handle verbal interaction issues, know when to start up a conversation, and which one, carry on multiple conversations/interrupted conversations, respond to client initiated interaction, extract information from spoken words, time stamp information, skip asking a question, continue a conversation at a later time or repeat a question.

A VHVI platform is an electronic device that is used as a platform to create a VHVI device. The platform contains all the core/common elements of a VHVI device. The device can include a computing device with Connections for a microphone and speaker, a microphone and speaker, voice recognition and speech synthesis capabilities, VHVI software programs, VHVI-based tables, such as for storing data, a database for storing IMPs/parameter values, other data structures and a device driver for microphone and speaker.

The purpose of the VHVI platform is to enable VHVI devices and systems to be quickly and easily developed, and deployed. A developer simply designs the custom data required by the platform to carry out the VHVI application. This data is loaded onto the platform. If other (non-CHVI) functionality is required, custom programs are created and added to the platform.

To build a VHVI device, based on the VHVI platform, a developer can perform the following steps: create detailed VHVI conversation specifications; convert the specifications into data for the various tables; load the data into the platform tables; and if required, develop custom software, and load the software onto the platform.

Specifically, a developer could use the following steps to create a platform.

1) Define all the computer-human conversations that the device is to be capable of having with a user, including creating a written specification for each conversation.

2) Define the trigger conditions associated with each conversation.

3) Define the priority of each conversation.

4) Define the user words, or phases, that the device is to recognize as triggers, for each trigger, specify the conversation that is to start up.

5) Define the IMPs.

6) Define the vocabulary of the device, as required for the application, including every word, and phrase, that the device is to understand and how the device is to interpret the word/phrase.

7) Define additional functionality, other than computer-human interaction functionality, required of the device, if any.

8) Convert conversation specifications into interaction session-formatted data.

For each conversation:

a) Break the conversation into its interactive units

b) For each interactive unit, define outgoing text (and OTS Instruction, if any), valid inputs, other conditions, actions to be taken and associated with each condition, interactive unit groups, IMP# and replay-max delay of each interactive unit.

c) Define the interactive session-level data, such as, too much time, unrecognizable words, non-valid input or non-understood input interactive session codes.

9) Convert trigger condition specs into probe trigger table and routine trigger table and emergency detection table data.

10) Determine data for client initiated interaction.

11) Determine data for a vocabulary table.

12) Load the above data into appropriate tables.

13) Establish data storage areas for each of the defined IMPs, in the parameter data storage area.

14) Create custom software to carry out the defined additional functionality, if any. The software links to the VHVI software by accessing the parameter data storage area.

15) Load the custom software onto the platform.

The types of information that is obtained from the client can be broken up into categories. When the system begins speaking to the client, the conversation can be to generally find out the general status of the client's health, safety or wellbeing. If the client responds to a question with a particular response or uses a word that indicates that there is a problem during the conversation, the system either immediately contacts emergency services or asks more questions to decide what to do. In addition to, or as an alternative to, using the words obtained from the client to make a decision how to proceed, the system can also use the quality of the client's response.

If after eliciting responses to obtain general information about the client, such as “Are you OK?” the system determines that there is a problem, or in response to receiving some other trigger event, the system can ask for responses that indicate a mental status or a physiological status of the client. These questions can be asked from specific scripts. If physiological status information or mental status information indicates that an emergency may be occurring or about to occur, the system can decide whether to wait and check back with the client or whether to contact emergency services. A physiological status question posed by the system may be, “What is your blood sugar level right now?”

Even if the physiological status information or mental status information from the client indicates that a there is no emergency, the system can ask questions that provide information regarding the client's safety. Such safety information can be information, such as “Do you need me to call the police?”

Either after obtaining general information from the client or instead of obtaining general information from the client, the system can provide educational information or reminder information to the client, such as “Today is election day” or “Did you remember to take your cholesterol medication this morning?”

The system can also obtain emergency information from the client, that is, the system can know when the client is calling for help or indicating that there is an emergency.

Because the system is computer based, it does not know on its own what type of questions to ask and what responses indicate whether the client is in good or bad health, is safe or in danger or is mentally incapacitated or mentally in good condition. The system must be instructed what questions to ask to obtain general information about the client, what to ask to obtain mental status information or physiological status information or safety information, or what statements to make to provide the client with educational information or reminder information. These different types of questions and statements, and the answers that the system is able to use to make determinations about how to proceed, are programmed into the system and can be updated to the system periodically, if desired.

Below the various data structures, tables and data stores that can be used with a system are described. Any feature described may be optional.

An ISD is a table that formally describes the interaction session. It contains the data that enables the system to carry out a verbal interaction. An ISD consists of some interactive session-related data, plus data associated with interactive units. The ISDs are saved in the ISD Store. Below is an example of an ISD:

TABLE 1

| | | | |
|---|---|---|---|
| IS# | 0555 | TMT-IS Action | <CALL IS#LOS-1/IU#600> |
| T- | 80 | URW-IS | <CALL IS#LOS-1/IU#700> |
| InterruptionMax | | Action | |
| RMD-IS | 0 | NVI-IS Action | <CALL IS#LOS-1/IU#800> |
| S-Time | 00 | NUI-IS Action | <CALL IS#LOS-1/IU#800> |

TABLE 2

| | | | | | | RMD- |
|---|---|---|---|---|---|---|
| | | Decision Statement | | IU | | IU |
| IU # | Output Text String | Condition | Action | Group | IMP# | (secs) |
| 10 | <NRR> | | <GOTO | | | |
| | Good morning, John. It's | | IU#20> | | | |
| | 9:00AM. | | | | | |
| 20 | This is just a quick health | OK | IU#30 | | 1 | 25 |
| | check-up. How do you | Not OK | IU#40 | | | |
| | feel? | | | | | |
| 30 | <NRR> | | <END | | | |
| | Good. I will check in | | SESSION> | | | |
| | with you later. | | | | | |
| 40 | <NRR> | | <END | | | |
| | I will call Emergency | | SESSION> | | | |
| | Response personnel right now. | | | | | |

The following describes each of the fields of an IS Definition.

### IS#:

This code uniquely identifies each interaction session, and its associated ISD.

### T-InterruptionMax:

Indicates how long this interactive session can be interrupted before it will automatically start over (in seconds).

### RDM-IS

This is the maximum length of time that the person has to reply to an OTS (in seconds).
This value will be used when there is not entry in the RDM-IU column associated with each interaction unit.

### S-Time

A value, in seconds, can be put into this field (optional).
When a value, x, is put into this field, the interaction sessions is in S Mode. S Mode operation deals with situations where a question is asked of the client, that was asked (and replied to) recently. For example, a client may indicate pain in a master interaction session. A heart attack interaction session may start up right away, and one of its first questions can be “Do you have pain?” In S Mode, when an interaction unit is initiated, it first checks the values and timestamps of the interaction-monitored parameters (IMP) associated with the interaction unit. If the client has given a value less than x seconds ago, then this value is used as the reply to the OTS. The action associated with this reply is carried out.
The purpose is to avoid asking the client the same question within a short period of time. The system therefore skips a question it already knows the answer to.

### TMT-IS Action

This is the action to be carried out if the too much time (TMT) code, indicating that the client has taken too long to reply, is received by an interaction unit, and the interaction unit does not have its own TMT Code Action.

### URW-IS Action

This is the action to be carried out if the unrecognizable words (URW) Code, indicating that the client is having trouble speaking, is received by an interaction unit, and the interaction unit does not have its own URW Code Action.

### NVI-IS Action

This is the action to be carried out if the non-valid input (NVI Code), indicating that the client has provided inappropriate words in reply to a query, is received by an interaction unit, and the interaction unit does not have its own NVI Code Action.

### NUI-IS Action

This is the Action to be carried out if the non-understood input (NUI) Code, indicating that the client has provided inappropriate words in reply to a query, is received by an interaction unit, and the interaction unit does not have its own NUI Code Action.

Each Interaction Unit in the interaction session contains the following fields: Interaction Unit (IU) #, Output Text String, which may include OTS Instruction(s), Decision Statement, which includes Condition and Action, IU Group, IMP #, RMD-IU (Reply-MaxDelay). These fields are described further below.

Interaction Unit (IU) #
A code that uniquely identifies the RI, e.g., IU#10

Output Text String (OTS)
The OTS indicates what the system communicates to the client.
This is the text string that is and “spoken” to the client or displayed on a screen to the client.
The OTS may contain OTS Instructions, as described further herein.

Decision Statement

The Decision Statement is executed when the system receives an input, in response to the OTS. The Decision Statement instructs the system as what to do next, based on how the client replied to the associated OTS. Often, the next step is the execution of another IU. The Decision Statement consists of several Conditions/Inputs and associated Actions.

Decision Statement—Conditions
The Condition List of the Decision Statement can contain three types of Conditions, the valid inputs associated with the OTS, special codes, such as a TMT—“Too Much Time” Code, a URW—“Unrecognizable Words” Code, including an NVI—“Non-Valid Input” Code and/or an NUI—“Non-Understood Input” Code, or special conditions, which are logical statements.

Action—Decision Statement
The action column contains one or more actions; each one is associated with an entry in the condition column.
When a condition is TRUE, the corresponding action is carried out.
The most common action is to execute another IU.

IU Group #
When two or more IU's are associated with a particular activity, they are given the same IU Group #. For example, three IU's may be associated with finding out if the client has numbness on one side of his/her body, if it happened suddenly, and if it is mild or serious.
The IU Group # is used when an ReIS is interrupted by another ReIS. When the second ReIS is finished, the interrupted ReIS is resumed, starting with the first IU of the IU Group associated with the IU that was interrupted.

IMP# (Interaction-Monitored Parameter #)
The IMP# is used to indicate whether the valid input is directly associated with an IMP, and if it is, what the # of the IMP is.

RMD-IU
This value indicates the maximum amount of time that the client has to reply, after the system has “spoken” something to the client.
The value is in seconds.

The ISs described above can allow the apparatus to handle various situations. For example, if the system asks the client a question and does not receive a valid response, the system can repeat the question a few times, repeat the question, plus say a list of acceptable replies to the question or determine that there is a problem and escalate the situation by testing the client's mental state or calling for help.

OTS Instructions

OTS Instructions are part of the OTS field, but they are not outputted to the client. An OTS Instruction is executed when the system is preparing to send out an OTS to the client. An OTS Instruction is stripped off and executed when it is encountered within the OTS, before the outgoing text, after the outgoing text, or within the outgoing text. An example of an OTS Instruction is: <PRESENT_TIME>. This instruction says: Get the present time, convert it into a text string, and insert it into the present OTS.

The following lists all the possible OTS Instructions that can be found in the OTS field of an IU, and a description of what each one does:

TABLE 3

| OTS Instruction | What It Does |
|---|---|
| <NRR> | Indicates that no reply is required. |
| | Execute the Action in the Decision Statement. |
| <GET Tx, Ty, TN> | Get the value(s) contained in the Tx, Ty, TN Temporary |
| | Registers of the Active ReIS Data Store, and insert the |
| | corresponding text into the OTS, at the position of the “<>” |
| | symbol. |
| <GET VALID INPUTS> | Get the text contained in the Previous Valid Input Registers |
| | of the Active ReIS Data Store. |
| | Insert this text in the OTS at the position of the “<>” |
| | symbol. |
| <S-OTS P#xxx yyys> | Access the latest Timestamp of each of the IMPs in this IU. |
| | Find the value that is the most recent. Check if this value |
| | was received less than yyy seconds ago. If Yes, then Skip |
| | the OTS - do not output the OTS. Go directly to the |
| | Decision Statement. Carry out the Action associated with |
| | the Valid Input, which is associated with the latest IMP |
| | value, determined above. If Not received less than yyy |
| | seconds ago, then carry on in regular fashion. |
| | Note 1: This OTS Instruction is utilized to avoid asking the |
| | client a question that was just asked of him/her very |
| | recently. |
| | Note 2: This OTS Instruction is only used if there is no |
| | value in the S-Time field. |
| <NO S-OTS> | Do not apply the S Mode of operation to this IU. |
| <NO OTS> | Indicates that the IU contains no OTS to send out to the |
| | client. Just carry out the Decision Statement. |
| <NAME> | Get the first name of the client, from the Client Information |
| | Table, and insert the corresponding text into the OTS, at the |
| | position of the symbol “<N>”. |
| <PRESENTTIME> | Get the present time, and insert the corresponding text into |
| | the OTS, at the position of the “<>” symbol. |
| <TELEPHONE# > | Get the telephone number for from the |
| | Telephone Database, and insert the corresponding text into |
| | the OTS, at the position of the “<>” symbol. |
| <COMMENT xxxxxxxx> | Ignore the following. (Do not execute.) |

Every time an OTS is processed, the first character of the OTS is reviewed to determine if it is a “<”, an OTS Instruction has been encountered. A “>” is then searched for. Everything between the <and > symbols are pulled from the OTS and is the OTS Instruction. The OTS Instruction is processed and sent out to be communicated to the client.

The following explains aspects of the Conditions in the Condition list:

Order of Condition Evaluation:
The Conditions listed in the Condition Column are evaluated, beginning with the first one and then going down the list.
If none of these Conditions evaluate “True”, then the IS-based Codes are evaluated.

<Other>
It is placed as the last Condition. If all the other Conditions are “False”, then the Action associated with <Other> is carried out. This Condition is optional.

I#xxx
This means to get the latest value of Parameter I#xxx.
Default: The value must have been obtained and saved in the DSA less than 60 seconds ago. If the value is older than 60 seconds, then a “NUL” value is returned.
I#xxx: Number of an IMP; P#xxx: Number of a PP; S#xxx: Number of an SMP: V#xxx: Number of a VMP.

I#xxx[zzzs]
This means to get the latest value of Parameter I#xxx.
The value must have been obtained less than zzz seconds ago. If the value is older than zzz seconds, then a “NUL” value is returned.

P#xxx[Ayys]
Get the value of Parameter, P#xxx, as of yy seconds ago.

I#xxx=V
Get the latest value of Parameter, I#xxx, and compare it to the value V.
If they are equal, then the condition is True. Otherwise, it is False.

TS(I#xxx)
Get the timestamp associated with the latest value of Parameter, I#xxx.

TA(P#xxx=N)
Number of seconds ago that Parameter, P#xxx, had a value of N.

TA(P#xxx)
Number of seconds ago that Parameter, P#xxx, was received.

P#xxx[hh::mm:ss]
The value of Parameter, P#xxx, at time hh:mm:ss.

N(P#xxx[Lyys]=X)
Number of times that Parameter, Pxxx, has value of X, over the last yy seconds.

N(P#xxx[Lyys])
Number of times that a value for Parameter, Pxxx, has been received, over the last yy seconds.

NI=xxx
This means to get the content of Register NI and to compare it with value xxx. If they match, then this Condition is “True”.

REGx=yyy
This means to get the content of Register REGx and to compare it with value yyy. If they match, then this Condition is “True”.

(Day of Week)
This is a variable that contains the present day of the week.

< >: Not equal

The following are the actions (or Action Instructions) that can be found in the “Action” field of an IU. These instructions are associated with a condition. An instruction is executed when the associated Condition is TRUE.

TABLE 4

| Action Instruction | What It Does |
|---|---|
| <GOTO IU#xxx> | Provides instructions to access a new IU (in the present IS) |
| or | with the # of xxx. The “GOTO” is optional. |
| <IU#xxx> | |
| Or | |
| Xxx | |
| <GOTO IS#yyy/IU#xxx> | Provides instructions to access a new IU with the # of xxx, |
| or | in the IS with # yyy. The “GOTO” text is optional. |
| <IS#yyy/IU#xxx> | |
| <CALL IU#xxx> | Like a <GOTO>, in that it provides instructions to access a |
| or | new IU (from the presently Active IS) with the # of xxx. |
| <C IU#xxx> | The difference is that when a <RETURN> is executed, the |
| | IU that follows the present IU is executed. |
| <CALL IS#xxx/IU#zzz> | Like a <GOTO>, in that it provides instructions to access a |
| or | new IU (in the IS with # xxx) with the # of zzz. The |
| <C IS#xxx/IU#zzz> | difference is that when a <RETURN> is executed, the IU |
| | that follows the present IU is executed. |
| <RETURN> | Provides instructions to access the IU that follows the IU |
| or | that <CALL>’ed. |
| <R> | |
| <RETURN-REPEAT> | Provides instructions to re-execute the IU from where the |
| or | CALL came from. |
| <RETURN-R> | |
| or | |
| <R-R> | |
| <END SESSION> | End the present Interaction Session. |
| or | |
| <END> | |
| or | |
| <E> | |
| <SAVE> | Save the associated Valid Input value in the Data Storage |
| or | Area of the IMP listed in the IMP# Column of the IU. Also |
| <S> | save the timestamp. |
| <SAVE “x”> | Save the value “x” in the Data Storage Area of the IMP |
| or | listed in the IMP# Column of the IU. Also save the |
| <S “x”> | timestamp. |
| <SAVE Tx> | Save the value contained in Temporary Register, Tx, in the |
| | Active ReIS data structure, in the Data Storage Area of the |
| | IMP listed in the IMP# Column of the IU. Also save the |
| | timestamp. |
| <TSAVE Tx>\|\| | Save the Valid Input value into the Temporary Register, |
| | Tx, in the Active ReIS Data Store. |
| <TSAVE Valid Inputs> | Save the Valid Inputs of the present IU in the Present Valid |
| | Inputs Register of the ReIS Data Store. |
| <Cx=Cx+1> | Increment the number in Register, Cx, in the Active ReIS |
| or | data structure. |
| Cx=Cx+1 | |
| <WAIT-zzzzS IS#yyy> | Activate IS#yyy in zzzz seconds from now, or at the time |
| or | of hh:mm:ss. |
| <WAIT-hh:mm:ss IS#yyy> | [Load the Activate Time into the Trigger Condition |
| | Description field of the Record associated with IS#yyy (in |
| | the PT Table or RT Table).] |
| <RxSave “yyy”> | Save “yyy” into Register REGx. |
| <NSAVE “yyy”> | Save “yyy” into Register NI |

Multiple actions can be associated with one condition. They can be separated by the symbol “∥” to indicate each separate action.

A system uses the IMP to condense information received from the client into values. The system can access the values immediately or in the future to make decisions. An IMP is a pre-defined parameter whose value, at any point in time, is determined, or measured, such as by asking the client to verbally reply to a statement or question. If the reply from the client has a valid value (i.e., the reply is one of the possible valid values associated with an IMP), the value is saved. An example of an IMP could be {Person is happy}. When the system asks the client if he is happy, the system condenses the reply into a value (Yes or No, in this case), and saves this value, under {Person is happy}.

Every parameter that is measured/monitored has an associated Data Storage Area assigned to it. This applies to physiological parameters (PPs), sound monitor parameters (SMPs), video monitored parameters (VMPs) and IMPs.

When a value for a parameter (PP, IMP, SMP, VMP) is received, or when a value is extracted for a parameter from an in-coming signal from a monitoring device, the value is saved in the DSA associated with that parameter, in some embodiments, along with a timestamp, e.g., 2006/April/6/14/34/20. This can be performed each time a new parameter value is received or extracted. New parameter values can be routinely or continuously checked for. The timestamp indicates the time that the parameter value was obtained. If the parameter values are received at regular time intervals or small time intervals, then the timestamp only has to be saved periodically. Also, when an IS is executing, and a value associated with an IMP is received, the value is saved in the DSA associated with that parameter. In addition, it saves a timestamp with the parameter value.

The system can use the timestamp to determine if new information is needed. For example, the system can make a decision that requires that the value of a certain IMP must have been obtained recently, say within the last hour. The system accesses the latest value of the IMP in memory, and checks the timestamp to determine if it is less than one hour old. If yes, then the system would uses the value in its decision-making process. If no, the system asks the client for a current value.

Another use for time stamping is to enable the apparatus to carry out analysis, or other actions, based on historical IMP values. For example, the system could ask the client how her headache is every half hour, and if it is getter better or worse. The system can then analyze the historical data and check if the headache is consistently getting worse, such as over the previous two hours. If yes, the apparatus can auto-alert emergency response personnel.

The IMP values, as well as other values, such as physiological parameter output values, can be used to weight an input. For example, a moderately temperature, such as 99.5° F., can cause the system to merely monitor the client, while a high temperature, such as 104° F. can cause the system to alert emergency services. The system can use the value to determine how serious the client's condition is when deciding whether to alert emergency services. Multiple values can be used in combination to decide whether to call for help.

Exemplary parameters are shown below in Tables 5-8. For each parameter, a parameter code, a parameter description and valid values are provided. A parameter code uniquely identifies the parameter. A parameter description is a short written description of the parameter. The valid values is a list of the values of the parameter that are supported or recognized.

The physiological parameters are stored in the same format as used with IMP values. This consistent parameter format enable the system to easily mix IMP values and physiological parameter output values in analysis.

### Physiological Parameter (PP) List

TABLE 5

| PP Code | PP Name | Valid Values |
|---|---|---|
| HL1E | Heart Rate - Low (Below Level 1) - ECG Monitor | Y; N |
| HL9E | Heart Rate - Low (Below Level 9) - ECG Monitor | Y; N |
| HH1E | Heart Rate - High (Above Level 1) - ECG Monitor | Y; N |
| HH9E | Heart Rate - High (Above Level 9) - ECG Monitor | Y; N |
| HL1M | Heart Rate - Low (below Level 1) - Heart Rate Monitor | Y; N |
| HL9M | Heart Rate - Low (Below Level 9) - Heart Rate Monitor | Y; N |
| HH1M | Heart Rate - High (Above Level 1) - Heart Rate Monitor | Y; N |
| HH9M | Heart Rate - High (Above Level 9) - Heart Rate Monitor | Y; N |
| HL1B | Heart Rate - Low (below Level 1) - Pulse Oximeter | Y; N |
| HL9B | Heart Rate - Low (Below Level 9) - Pulse Oximeter | Y; N |
| HH1B | Heart Rate - High (Above Level 1) - Pulse Oximeter | Y; N |
| HH9B | Heart Rate - High (Above Level 9) - Pulse Oximeter | Y; N |
| RL1E | Respiratory Rate - Low (Below Level 1) - ECG Monitor | Y; N |
| RL9E | Respiratory Rate - Low (Below Level 9) - ECG Monitor | Y; N |
| RH1E | Respiratory Rate - High (Above Level 1) - ECG Monitor | Y; N |
| RH9E | Respiratory Rate - High (Above Level 9) - ECG Monitor | Y; N |
| RL1B | Respiratory Rate - Low (Below Level 1) - Pulse Oximeter | Y; N |
| RL9B | Respiratory Rate - Low (Below Level 9) - Pulse Oximeter | Y; N |
| RH1B | Respiratory Rate - High (Above Level 1) - Pulse Oximeter | Y; N |
| RH9B | Respiratory Rate - High (Above Level 9) - Pulse Oximeter | Y; N |
| BOL1 | Blood Oxygen Saturation - Low (Below Level 1) | Y; N |
| BOL9 | Blood Oxygen Saturation - Low (Below Level 9) | Y; N |
| TEL1 | Temperature - Low (Below Level 1) | Y; N |
| TEL9 | Temperature - Low (Below Level 9) | Y; N |
| TEH1 | Temperature - High (Above Level 1) | Y; N |
| TEH9 | Temperature - High (Above Level 9) | Y; N |
| FDM | Fall Detection Monitor has detected a fall. | Y; N |
| HRE | Heart Rate [ECG Monitor] | 1-250/min |
| HRP | Heart Rate [Pulse Oximeter] | 1-250/min |
| HRM | Heart Rate [Heart Rate Monitor] | 1-250/min |
| TEM | Body Temperature | 1-200 C. |
| BP | Blood Pressure | 1-200 |
| RR | Respiratory Rate | 0.1-200 per |
| | | minute |
| BOS | Blood Oxygen Saturation | 0-100% |
| BG | Blood Glucose Level | Standard |
| | | Range |
| AF | Atrial Fibrillation Heart Condition | Y; N |

### Interaction-Monitored Parameter (IMP) List

TABLE 6

| | | Valid Values/ |
|---|---|---|
| IMP Code | IMP Description | Inputs |
| NU | {Client says that has sudden numbness} | Yes; No |
| NUL | {Client says that has numbness in this location} | Arm; Leg; Face; Other |
| NAR | Numb arm location | Left; Right; Both; Y; N |
| NLE | Numb leg location | Left; Right; Both; Y; N |
| NFA | Numb Face/Mouth location | Left; Right; Both sides; |
| | | Y; N |
| NSI | {Client says that numbness is on this side} | Left; Right |
| N1S | Numbness on one side? | Yes; No; Not sure |
| WE | {Client says that has sudden weakness} | Yes; No |
| WEL | {Client says that has weakness in this location} | Arm; Leg; Face; Other |
| WAR | Weak arm location | Left; Right; Both; Y; N |
| WLE | Weak leg location | Left; Right; Both; Y; N |
| WFA | Weak Face/Mouth location | Left; Right; Both sides; |
| | | Y; N |
| W1S | Weakness on one side? | Yes; No; Not sure |
| WSI | {Client says that weakness is on this side} | Left; Right |
| WES | Weakness severe | Yes; No |
| WEB | {Client says weakness is bad} | Yes; No |
| WECW | {Client says weakness is so bad that can't walk} | Yes; No |
| AD1 | Result of “Arm Drift” Test - | Yes; No |
| | One arm comes down faster than the other. | |
| AD2 | Result of “Arm Drift” Test - | Yes; Left; Right |
| | Which arm comes down faster than the other. | |
| ST1 | Result of “Smile” Test - Client has problem to | Yes; No; Not sure |
| | smile. | |
| ST2 | Result of “Smile” Test - Does face/mouth droop. | No; Yes |
| ST3 | Result of “Smile” Test - Which side does it droop, | Left; Right; Both |
| | or both sides. | |
| F1S | Droopy on one side of face/mouth? | Y; N |
| PA | {Client says he/she in pain} | Yes; No |
| PCH | {Client says pain in chest} | Yes; No |
| PCC | {Client says pain in center of chest} | Yes; No |
| PS | {Client says pain is steady or comes and goes} | Steady |
| | | Not steady |
| PG5 | {Client says pain had lasted for more than 5 | Yes; No |
| | minutes} | |
| PAB | {Client says pain is bad} | Yes; No |
| PACW | {Client says pain is so bad that can't walk} | Yes; No |
| DI | {Client says in discomfort} | Yes; No |
| DCC | {Client says discomfort in center of chest} | Yes; No |
| DT | {Client says the type of discomfort} | Pressure; Fullness; |
| | | Squeezing |
| DS | {Client says discomfort is steady or comes and | Steady; Not steady |
| | goes} | |
| DG5 | {Client says discomfort had lasted for more than 5 | Yes; No |
| | minutes} | |
| OK | {Client says that feels OK} | Yes; No; Not sure |
| OK1 | {Client's response to: “How do you feel?”} | Good; Bad; In Between |
| TW1 | Trouble walking | Yes; No; Somewhat |
| FS1 | Feel “Strange” | Yes; No; Somewhat |
| FS2 | Feel Funny | Yes; No |
| FS3 | Something's Wrong | Yes; No |
| FS4 | Doesn't Feel Right | Yes; No |
| FCH | Feel “strange” - Chest | Yes; No |
| FBA | Feel “strange” - Back | Yes; No |
| FNE | Feel “strange” - Neck | Yes; No |
| FJ | Feel “strange” - Jaw | Yes; No |
| FST | Feel “strange” - Stomach | Yes; No |
| FSH2 | Feel “strange” - Shoulders | Yes; No |
| FSH1 | Feel “strange” - One shoulder | Yes; No |
| FA2 | Feel “strange” - Both arms | Yes; No |
| FA1 | Feel “strange” - One arm | Yes; No |
| FH | Feel “strange” - Head | Yes; No |
| FFA | Feel “strange” - Face | Yes; No |
| FL1 | Feel “strange” - One leg | Yes; No |
| FSB | Feel “strange” - Bad | Yes; No |
| FSCW | Feel “strange” - And can't walk | Yes; No |
| RV | {Client is responsive - Verbally} | Yes; No |
| RVS | {Client is responsive - Vocal sounds} | Yes; No |
| RKS | {Client is responsive - Making knocking sounds} | Yes; No |
| RAW | {Client is responsive - Waving arm} | Yes; No |
| RLR | {Client is responsive - Lifting leg} | Yes; No |
| RAS | {Client is making random vocal sounds} | Yes; No |
| EQE | {Client says that he/she is OK, but physiological | Yes; No |
| | parameter values indicate a health problem.} | |
| EQG | {Equipment is operating OK, per client} | Yes; No |
| TS1 | {Client has trouble speaking) | Yes; No; Somewhat |
| DOS | [Working on S-1] | Yes; No |
| DOHA | [Working on HA-1] | Yes; No |
| DOCA | [Working on CAE-1/CAO-1] | Yes; No |
| M1DO | [Go to IS#M-1] | Yes; No |
| EM1 | {Client says, “Emergency”} | Yes; No |
| EM2 | {Client says, “Help”} | Yes; No |
| EMC | An Emergency-Caution from the Control Unit. | Yes; No |
| EM4 | Client indicates an Emergency - Client can't speak - | Yes; No |
| | Emergency indicated by non-verbal means. | |
| EM5 | Control Unit decides to make an Emergency call | Yes; No |
| EMN | Control Unit decides to make an Emergency call - | Yes; No |
| | Client says “Emergency Now”. | |
| EMG | General Emergency, per client. | Yes; No |
| EMCM | Emergency - Client says can't move. | Yes; No |
| EMCW | Emergency - Client says that can't walk. | Yes; No |
| FCU | Client says “I fell, and I can't get up”. | Yes; No; Not sure |
| FA | Client says, “I fell”. | Yes; No |
| FTL | Client fell, and took too long to get up. | Yes; No |
| CM1 | Client says “Can't move” | Yes; No |
| CM2 | Client says “Can't walk” | Yes; No |
| CH | Client says “Chest” | Yes; No |
| HE | Client says “Heart” | Yes; No |
| BR1 | Breathing problem | Yes; No; Mild; |
| | | Moderate; Serious; |
| | | Severe |
| BRS | Shortness of breath | Yes; No; Mild; |
| | | Moderate; Serious; |
| | | Severe |
| NA1 | Nauseous | Yes; No |
| IL | Client says “I'm ill/sick” | Yes; No |
| ICH | Ill - Chest | Yes; No |
| IH | Ill - Head | Yes; No |
| IST | Ill - Stomach | Yes; No |
| IAL | Ill - All over | Yes; No |
| ILB | {Client says illness is bad} | Yes; No |
| ILCW | {Client says illness is so bad that can't walk} | Yes; No |
| LBA | Loss of Balance | Yes; No |
| LCO | Loss of Coordination | Yes; No |
| EP | Eye Problem | Yes; No |
| PCH | Pain - Chest | Yes; No; Mild; |
| | | Moderate; Serious |
| PH | Pain - Head | Yes; No; Mild; |
| | | Moderate; Serious |
| PHE | Pain - Heart | Yes; No; Mild; |
| | | Moderate; Serious |
| PBA | Pain - Back | Yes; No |
| PST | Pain - Stomach | Yes; No |
| PNE | Pain - Neck | Yes; No |
| PSH1 | Pain - Shoulder | Yes; No |
| PSH2 | Pain - Shoulders | Yes; No |
| PJ | Pain - Jaw | Yes; No |
| PFA | Pain - Face | Yes; No |
| PA1 | Pain - Arm | Yes; No |
| PA2 | Pain - Arms | Yes; No |
| PL1 | Pain - Leg | Yes; No |
| PL2 | Pain - Legs | Yes; No |
| PSE | Pain - Severe | Yes; No |
| DCH | Discomfort - Chest | Yes; No; Mild; |
| | | Moderate; Serious |
| DH | Discomfort - Head | Yes; No; Mild; |
| | | Moderate; Serious |
| DHE | Discomfort - Heart | Yes; No; Mild; |
| | | Moderate; Serious |
| DBA | Discomfort - Back | Yes; No |
| DST | Discomfort - Stomach | Yes; No |
| DNE | Discomfort - Neck | Yes; No |
| DSH1 | Discomfort - Shoulder | Yes; No |
| DSH2 | Discomfort - Shoulders | Yes; No |
| DJ | Discomfort - Jaw | Yes; No |
| DFA | Discomfort - Face | Yes; No |
| DA1 | Discomfort - Arm | Yes; No |
| DA2 | Discomfort - Arms | Yes; No |
| DL1 | Discomfort - Leg | Yes; No |
| DL2 | Discomfort - Legs | Yes; No |
| DICW | Discomfort, and Can't Walk | Yes; No |
| DIB | Discomfort - That is Bad | Yes; No |
| PEY1 | Pain - One eye | Yes; No |
| PEY2 | Pain - Two eyes | Yes; No |
| DI | Discomfort | Yes; No |
| DI1 | Discomfort - Pressure | Yes; No |
| DI2 | Discomfort - Fullness | Yes; No |
| DI3 | Discomfort - Squeezing | Yes; No |
| CW | {Client says that can't walk} | Yes; No |
| UNC | {Control Unit determines that client is | Yes; No |
| | Unconscious} | |
| LRM | {Control Unit determines that client has Loss of | Yes; No |
| | Responsiveness, but is moving} | |
| LRU | {Control Unit determines that client has Loss of | Yes; No |
| | Responsiveness, and movement is unknown} | |
| EMCS | {Client indicates that he/she needs help, or that the | Yes; No |
| | situation is “Bad” or is an Emergency} | |
| BVR | “Bad” verbal response - Client is not responding to | Yes; No |
| | questions with valid inputs, after several attempts} | |
| UT | Result of the “Understanding” Test. | Pass; Fail |
| DIZ | Dizzy | Yes; No |
| HA | Headache | Yes; No |
| LH | Lightheaded | Yes; No |
| CS | Cold Sweat | Yes; No |
| AT | {Client says, “Attention”} | Yes; No |
| ED | {Client says, “Ed”} | Yes; No |
| EDI | {Client says, “Edie”} | Yes; No |
| FD1 | {Client says, “Face is droopy”} | Yes; No |
| FD2 | {Client says, “Mouth is droopy”} | Yes; No |
| EQP1 | {Client having problem with equipment} | Yes; No |
| PSVY | {Client verbally confirms that he/she just made a | Yes; No |
| | cry of pain} | |
| FSVY | {Client verbally confirms that he/she just fell} | Yes; No |
| PP | {Indicates that a Physiological Parameter Threshold | Yes; No |
| | value has been reached, and that control is coming | |
| | from IS#MPP-1.} | |
| SMP | {Indicates that control is coming from IS#MS-1.} | Yes; No |
| VMP | {Indicates that control is coming from IS#MV-1.} | Yes; No |

### Sound-Monitored Parameter (SMP) List

TABLE 7

| SMP | | Valid |
|---|---|---|
| Code | SMP Description | Values |
| PAS1 | {Cries of pain} | Y: N |
| PAS2 | “Ouch” | Y; N |
| S2 | Sound of a person gasping for air. | Y; N |
| FAS1 | Sound of falling | Y; N |
| S5 | {Crying} | Y; N |
| S7 | {Bumping into furniture} | Y; N |
| S8 | {Glass breaking} | Y; N |
| S9 | {Loud bang on wall/floor} | Y; N |
| KS1 | One knocking sound, and no knocking sound for at | Y; N |
| | least 7 seconds after that (from the client). | |
| KS2 | Two knocking sounds, within 5 seconds, and no | Y; N |
| | knocking sound for at least 7 seconds after that | |
| | (from the client). | |
| KS3 | Three knocking sounds, within 10 seconds, and no | Y; N |
| | knocking sound for at least 7 seconds after that (from | |
| | the client). | |
| YS1 | One “yelp” sound, and no “yelp” sound for at | Y; N |
| | least 7 seconds after that (from the client). | |
| YS2 | Two “yelps”, within 5 seconds, and no “yelp” sound | Y; N |
| | for at least 7 seconds after that (from the client). | |
| YS3 | Three “yelps”, within 5 seconds, and no “yelp” | Y; N |
| | sound for at least 7 seconds after that (from the client). | |
| EMK | Special knocking sequence to indicate Emergency: 2 | Y; N |
| | knocks - pause - 2 knocks, within 15 seconds. | |
| EMY | Special yelping sequence to indicate Emergency: 2 | Y; N |
| | yelps - pause - 2 yelps, within 15 seconds. | |
| SY | Client has made a sound that indicates: “Yes” | Y; N |
| SN | Client has made a sound that indicates: “No” | Y; N |
| SMP1 | Client confirmed that he/she made cry of pain. | Y; N |
| SMP2 | Client confirmed that he/she said “Ouch”. | Y; N |
| SMP3 | Client confirmed that he/she fell, after having made a | Y; N |
| | “fall” sound. | |

When an SMP is detected, an SMP Detected flag can be set, identifying the SMP in an SMP # Register. The value of the SMP can also be placed in the SMP Register. When a set “SMP Detected” Flag is detected, which SMP it is can be determined from the “SMP #” Register. The SMP value is grabbed from the SMP Register, and saved in the DSA of the SMP, along with the timestamp.

For example, the sound of glass breaking can be detected—loud for 2 seconds and moderate for 2 seconds, starting at 8:03:10 PM. A SMP Handling Routine can access the DSA of this SMP: {Glass breaking}, and store the following data:
Loud-05/10/10/20:03:10
Loud-05/10/10/20:03:11
Moderate-05/10/10/20:03:12
Moderate-05/10/10/20:03:13

Video-Monitored Parameter (VMP) List

TABLE 8

| VMP Code | VMP Description | Valid Values |
|---|---|---|
| FAV | Client Falling | Y; N |
| TWV | Client stumbling while walking | Y; N |
| LYV | Client lying down in the room | Y; N |
| DF1V | Face droopy | Y; N |
| DF2V | Mouth droopy | Y; N |
| MO | This parameter is “Yes” whenever the Video Monitor | Y; N; Unknown |
| | Detects the client moving; “No” when client comes into | |
| | view, stays in view, and stops moving; “Unknown” | |
| | when client is not in view of the Video Monitor. | |
| AW1 | Client waves arm once, and no waving for at least 10 | Y; N |
| | seconds after that. | |
| AW2 | Client waves arm twice, within 15 seconds, and no | Y; N |
| | waving for at least 10 seconds after that. | |
| AW3 | Client waves arm three times, within 20 seconds, and no | Y; N |
| | waving for at least 10 seconds after that. | |
| LR1 | Client lifts leg once, and no leg lifted for at least 10 | Y; N |
| | seconds after that. | |
| LR2 | Client lifts leg twice, within 15 seconds, and no leg | Y; N |
| | lifted for at least 10 seconds after that. | |
| LR3 | Client lifts leg 3 times, within 20 seconds, and no leg | Y; N |
| | lifted for at least 10 seconds after that. | |
| EMW | Special arm waving sequence to indicate Emergency: 2 | Y; N |
| | waves - pause - 2 waves, within 15 seconds. | |
| EML | Special leg lifting sequence to indicate Emergency: 2 | Y; N |
| | lifts - pause - 2 lifts, within 15 seconds. | |
| VY | Client has made a motion (e.g., arm wave) that | Y; N |
| | indicates: “Yes” | |
| VN | Client has made a motion (e.g., arm wave) that | Y; N |
| | indicates: “No” | |

In some systems, the video can capture a client performing a test to indicate whether the client is experiencing a particular problem. For example, an arm drift test can be used to determine whether client has had a stroke. The system can ask the client to hold a tennis ball in each hand and hold his hands at the same level. The system can train on the tennis balls and determine if the client lowers one of the tennis balls faster than the other, possibly indicating a stroke. In some embodiments, the system can capture when a client has not moved across the room for some specified amount of time, such as an hour. This lack of movement can be used as a trigger event.

When a VMP is detected, a VMP Detected Flag is set, identifying the VMP in a VMP # Register. A value of the VMP is also placed in the register. When a set “VMP Detected” Flag is detected, which VMP it is can be determined from the “VMP #” Register. The VMP value is then grabbed from the VMP Register, and saved in the DSA of the VMP, along with the timestamp.

For example, at 7:43:30 AM, the left side of the client's face is slightly droopy. Then, 30 minutes later, the client's face is significantly droopy. The DSA of the VMP: {Client's face is droopy}, can be accessed to store the following data:
Slightly-05/10/10/07:43:30
Significant-05/10/10/08:13:30

A requested IS is an IS to be carried out. As part of this process, a request is made and one of the ReIS DSs is allocated to the requested IS. In some embodiments, three Requested Interaction Session Data Stores (ReIS DS #1, #2, #3) are associated with requested IS, however fewer or more ReIS DSs could be associated with the IS. The data stores are used to hold temporary data while an ReIS is being executed, or while an ReIS is waiting to be carried out.

Data associated with the IS is loaded into one of these data stores. As the IS is executed, intermediate data is loaded into, and read from, portions of the ReIS DS. There can be one Active RIS, i.e., an ReIS that is being executed, as well as up to two ISs that could be waiting to be executed. An ReIS that is next in line to be carried out is an RIS-in-Waiting. It will be executed once the presently Active RIS is finished. An RIS-in-Waiting-2 is an ReIS that will be carried out after the RIS-in-Waiting is executed.

An IS Status field associated with each of the three data stores is used to handle multiple requests for IS. If there is a request for a new IS, and there is no active IS, then the new IS is made active, and its associated IS Status is set to “Active”. If a new IS Request comes in, while there is an Active IS, IS priority will determine which IS is given Active Status, and which gets “2” Status (IS-in-Waiting). If a new IS request comes in, and there already exists an Active ReIS, and an ReIS-in-Waiting, then IS Priority determines which IS is given Active Status, which gets IS-in-Waiting Status, and which gets IS-in-Waiting-2 Status.

Table 9 shows the fields contained in each Requested IS Data Table.

TABLE 9

| Field Name | RIS DS #1 | RIS DS #2 | RIS DS #3 |
|---|---|---|---|
| IS Status | | | |
| IS Interrupted | | | |
| IS # | | | |
| T-InterruptMax | | | |
| IS Interruption Time | | | |
| RMD-IS | | | |
| TMT-IS Action | | | |
| URW-IS Action | | | |
| NVR-IS Action | | | |
| NUI-IS Action | | | |
| IU # | | | |
| IU Group# | | | |
| IMP # | | | |
| RMD-IU | | | |
| OTS | | | |
| OTS-V Done | | | |
| OTS-SK Done | | | |
| Condition #1 | | | |
| Action #1 | | | |
| Condition #2 | | | |
| Action #2 | | | |
| Condition #3 | | | |
| Action #3 | | | |
| . . . | | | |
| . . . | | | |
| Condition #40 | | | |
| Action #40 | | | |
| T1 | | | |
| T2 | | | |
| T3 | | | |
| . . . | | | |
| T20 | | | |
| C1 | | | |
| C2 | | | |
| C3 | | | |
| . . . | | | |
| C20 | | | |
| Previous IU | | | |
| Valid Input #1 - of Previous IU | | | |
| Valid Input #2 - of Previous IU | | | |
| . . . | | | |
| Valid Input #30 - of Previous IU | | | |
| Call Return Register #1 | | | |
| Call Return Register #2 | | | |
| Call Return Register #3 | | | |
| Call Return Register #4 | | | |

REG#1, REG#2 . . . REG#10, NI Register and CIF Flag are external to and shared between the RIS DS#1, RIS DS#2 and RIS DS#3.

The fields that have not been previous described are described below.

### IS Status

If there is no Requested IS in this ReIS DS, the status is “Empty”

If there is a ReIS in the ReIS DS, then the status will be either: “Active”; “IS-in-Waiting”; “IS-in-Waiting-2”

### IS Interrupted

Was this ReIS interrupted: Yes or No

### IS Interruption Time

The time that this ReIS was interrupted

### OTS-V Done/OTS-SK Done

The time that a Text-to-Speech Routine (or Text Output Routine) finished outputting the OTS to the client.

### Previous IU

The # of the IU that was just executed.

Valid Input #x—of Previous IU

The Valid Inputs associated with the previous IU are held in these registers

### CALL Return Register #1-4

A CALL Return Register is used when executing a “CALL” Action. The # of the IS and IU to where the “CALL” is to return is placed here. The IS# is the number of the present IS. The RJ# is the # of the next RI in sequence.

There are four Registers to handle a “CALL within a CALL” situation.

The IS# and IU# are put into the first unoccupied register, starting from 1 and going up.

The IS# and IU# are retrieved from the first occupied register beginning from 4 and going down.

### REG#1 to REG#10

These registers are used by ISs to pass data among themselves.

### NI Register

When a Valid Input is received, the Valid Input is put into this register.

When a Client-Initiated Interaction input is received from the client, the input is put into this register.

### CIF Flag

This Flag is set when Client-Initiated Interaction input is received.

A Record for every Probe Trigger (PT) Condition that is recognized can be stored in a probe trigger table. Included in the table are records associated with client-initiated interactions that are probing type. A PT Condition is a condition that, if True, results in the start up of a probing IS. Each of the table records consists of the following fields: probe trigger (PT) condition, pointer to the IS (“conversation”) that is to be started up if the PT condition is True, PT priority and a PT record #.

Table 10 shows the structure, and the data fields, of the PT Table (also shown is some sample data):

TABLE 10

| | | | | | “Currently |
|---|---|---|---|---|---|
| | | | | Interaction | Being |
| | PT | PT Condition | | Session | Addressed” |
| PTC | Priority | Description | PT Condition | (IS) # | Flag |
| PT#10 | P1 | {Client has | I#NUL=Arm | IS#P10 | |
| | | numbness in arm} | | | |
| PT#500 | P7 | {Client calls out for | CII#100 | IS#500 | |
| | | help.} | | | |
| PT#999 | P4 | | {Time = hh:mm:ss} | IS#aaa | |
| | | | [See Note 1] | | |

Each Record in the Table contains the following data fields:

### PTC

A code that uniquely identifies the Probe Trigger

### PT Priority

This is a number that indicates the priority of a PT Condition, relative to all the other Trigger Conditions (PTCs and RTCs).
“1” is lowest priority, “9” is highest.
“P” is higher priority than “R”

### PT Condition Description

This is a basic text description of the PT Condition.

### PT Condition

The PT Condition is an entity that is evaluated. When the entity is evaluated as TRUE, the PT Condition is said to have occurred.
The entity can be one of three types
   Logical Statement
      A Logical Statement consists of Parameters, values, and Logical Operators. When the Logical Statement is TRUE, the PT Condition is said to have occurred.
      Example: {Heart Rate >100}
   PT Condition Pointer (See Note 2 below)
      The PT Condition Pointer points to a small subroutine in the Trigger Condition Store.
      When the outcome of the subroutine is TRUE, the PT Condition is said to have occurred. (The subroutine sets the “Condition True” Flag.)
   CII#
      The CII# refers to a particular Record in the client-initiated interaction condition (CIIC) table.
      When the CIIC Flag in that Record is “Set”, the PT Condition is said to have occurred.

### Interaction Session #

This is a code that uniquely identifies the Interaction Session that is to be carried out if the associated PT Condition is TRUE.

### “Currently Being Addressed” Flag

This flag is set when the Interaction Session associated with P-Trigger is being carried out.

This Record is associated with a <WAIT>Action. Normally hh:mm:ss is blank. When the associated <WAIT> Action is carried out, a time (Activate Time) is entered into hh:mm:ss. When this time arrives, this PT Condition will become TRUE, and IS#aaa will be executed.

Sometimes a PT Condition is too complex to be defined in a simple Logic Statement. When this happens, the Condition is defined in a TC Subroutine, that is stored in the Trigger Condition Store. The PT Condition Pointer is used by the TCAM to go to a particular TC Subroutine in the Trigger Condition Store, and execute the Subroutine.

A routine trigger (RT) condition specifies when the apparatus is to carry out a routine probe conversation. Routine probe conversations are initiated so that the information obtained from the conversation is optimized so that the client is not contacted too often and annoy the client or too infrequently so that the system fails to determine that there is a problem in a timely manner. RT conditions can be customized to the client, particularly the time that the conversations take place and how often. Some clients are awake early in the morning and can engage in an interaction early in the morning and are asleep in the early evening and should not be disturbed. Further, the RT conditions can be based on the client's SHE risk level, and on the client's tolerance for computer-human conversations.

An RT condition is a logic statement that consists of parameters, such as IMPs and time, logic operators and constants. An RT condition is a condition that, if True, results in the start up of a routine IS. Each of the Table records consists of the following fields: routine trigger (RT) condition, pointer to the IS (“conversation”) that is to be started up if the RT condition is True, RT priority and an RT record #.

A record for every RT condition that is recognized is stored in a Routine Trigger table. Included in the Table are Records associated with CII's that are “Routine” type.

Table 11 shows the structure, and the data fields, of the RT Table (also shown is some sample data):

TABLE 11

| | | | | | “Currently |
|---|---|---|---|---|---|
| | | | | Interaction | Being |
| | RT | RT Condition | | Session | Addressed” |
| RTC | Priority | Description | RT Condition | (IS) # | Flag |
| RT#10 | R5 | The time is 9 | {Time = 9:00 AM} | IS#062 | |
| | | am. | | | |
| RT#60 | R9 | Client wants to know | CII#001 | IS#120 | |
| | | the present time. | | | |
| RT#999 | R4 | | {Time = hh:mm:ss} | IS#zzz | |
| | | | [See Note 1] | | |

The data fields in the RT Table are all equivalent to the data fields in the PT Table.

An Emergency Detection (ED) Table contains a list of all the Emergency Conditions. An Emergency Detection Condition is a formal description of an emergency situation, a situation where there is a high probability that the person is experiencing the early warning signs, or occurrence, of an emergency situation. The Condition is described as a logical statement. It consists of parameters, values and logical operators (OR, AND, etc.). An example of a Condition that describes an Emergency situation is:

{Heart Rate<5 per sec.} AND {Client not responding>60 sec.}

Table 12 shows the structure, and the data fields, of the ED Table (also shown is some sample data):

TABLE 12

| | | | ED |
|---|---|---|---|
| | | | Interaction |
| | ED Condition | | Session |
| EDC | Description | ED Condition | (IS) # |
| E#0101 | Detection of Cardiac | (HR <20/min for >20 secs) | EIS# |
| | Arrest - | AND ((No Response OR | 0100 |
| | Heart Rate is very low, | “Bad Response” OR | |
| | and no response or | (Serious Situation - Per | |
| | “bad” response from | Client)) | |
| | client | | |

Each Record in Table 12 contains the following data fields:

### EDC

A code that uniquely identifies the Emergency Detection Condition, e.g., ED#100

### ED Condition Description

This is a basic text description of the ED Condition.

### ED Condition

The ED Condition is an entity that is evaluated. When the entity is evaluated as TRUE, the ED Condition is said to have occurred.

The entity can be one of two types

Logical Statement
A Logical Statement consists of Parameters, values, and Logical Operators. When the Logical Statement is TRUE, the PT Condition is said to have occurred.
Example: ({Sudden Numbness In Arm} AND {Duration of Numbness>5 minutes})

ED Condition Pointer (See Note 1 Below)
The ED Condition Pointer points to a small subroutine in the Data Store.
When the outcome of the subroutine is TRUE, the EDT Condition is said to have occurred.

### Interaction Session #

This is a code that uniquely identifies the Interaction Session that is to be carried out if the associated EDT Condition is TRUE.

Sometimes an ED Condition is too complex to be defined in a simple Logic Statement. When this happens, the Condition is defined in a TC Subroutine, that is stored in the Trigger Condition Store. The ED Condition Pointer is used to go to a particular TC Subroutine in the Trigger Condition Store, and execute the Subroutine.

When the system communicates with the client, the system is prepared to respond to anticipated replies from the client. These replies are called Valid Inputs/Replies. Sometimes the client will say something that is not in response to the query. The client may say something “out of the blue”, or the client may say something during an IS, that is not associated with the IS. For example, during an IS, when the system is asking how the client feels, the client may suddenly say, “What time is it?” or “Ouch, I just got a sharp pain in my chest.” These are called Client-Initiated Interactions (CII). To handle these CII situations, the system has a CIIC Table.

The CIIC Table has a Record for every CII situation that the system supports. Every Record includes a CII Condition. A CIIC is a logical statement made up of spoken words and logical operators. An example of a CIIC is: {“What” AND “time”}. When the CII Condition is found to be True, the associated Flag is set. (The VIHM evaluates these Conditions.)

Table 13 shows the structure, and the data fields, of the CIIC Table (also shown is some sample data):

TABLE 13

| | | | | |
|---|---|---|---|---|
| CII # | CII Condition Description | CII Condition | IMP | CIIC Flag |

| | | | |
|---|---|---|---|
| CII#001 | {Client says that | Have AND | PA-Y |
| | has pain} | Pain | |

Each Record in Table 13 contains the following data fields:

### CII #

Uniquely identifies the CII

### CII Condition Description

Describes the CIIC in words

### CII Condition

A CIIC is a logical statement made up of spoken words and logical operators. An example of a CIIC is: {“What” AND “time”}.
This explicitly lists the words, or word combinations, that when spoken by the client, are interpreted as a True CII Condition.

### IMP

If the CII is associated with an IMP, this Column is used.
The format is as follows:
   zzz-ttt, where zzz is the # of the IMP, and ttt is the value that is to be put into the DSA of the IMP.
Note: The timestamp is also stored with the value

### CIIC Flag

When the CII Condition is found to be True, this Flag is set.
It indicates that the system is presently addressing the Condition.

A verbal vocabulary and interpretation (VV&I) table defines the vocabulary used by the system. The Vocabulary is the list of words, and word groups, that the system understands and knows how to respond when these word(s) are spoken. The VV&I table (Table 53) also indicates how it interprets the words that are spoken by the client. For every word, or word group, that is spoken by the client, the Table indicates/shows how the system interprets it. The VV&I Table is used by the VIHM to interpret what the client said. The entries in the VV&I Table can be added to, modified or removed, if required. This can be done by an Administrator.

Table 14 shows the structure, and the data fields, of the VV&I Table (also shown is some sample data):

TABLE 14

| Vocabulary | Recognized Spoken Words |
|---|---|
| Yes | Yes; Sure; OK |
| No | No |

(A word combination is defined with logical operators; e.g., “Need AND Help”.)

A client information table holds medical information on the client. The system can use this information to properly analyze the client's health status for early warning signs, and occurrences, of SHEs. For example, a client may have poor balance, in general. The system needs to be able to factor this in when it is carrying out SHE monitoring, e.g., after having detected the client suddenly stumbling.

TABLE 15

| | |
|---|---|
| Client Field | Value/Status |

Client's Name
Home town
Street
Street number
Normally does not have trouble walking?
Normally, client's eyesight is OK with
glasses?
First name of client's first daughter
First name of client's second daughter
First name of client's first son
First name of client's second son
AND OTHERS

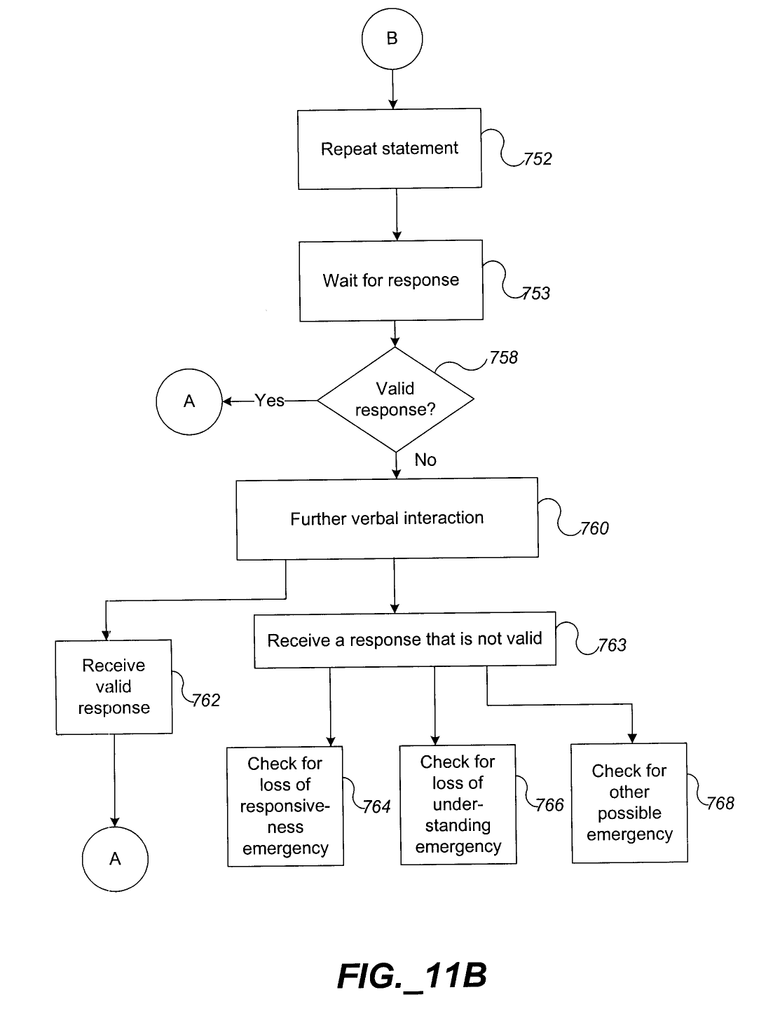

Referring to FIGS. 11A and 11B, the system can use ISs and various scripts to determine the client's status using the following method. The system initiates verbal interaction with the client (step 705). The system then makes a first statement, such as a question or a command (step 711) and waits for a response (step 713). Either the client does not respond, responds or does not respond with a predetermined time, such as 30 seconds or a minute. The system receives the response or lack thereof and determines whether the response is received within the predetermined time or not (step 720). If the response is not received within the predetermined time, the response is considered to be a delayed response. Receiving no response can also be categorized as a delayed response. If the response is received within the predetermined time, the system determines the quality of the response (step 730). The quality of the response can be one of valid, non-valid, not understood or not in the system's vocabulary. If the response is valid and has an IMP value, the IMP value, along with an optional timestamp, can be saved in memory (step 732). The system determines whether there are more statements to be made to the client (step 735). If there are no more statements, the IS ends. If there are more statements, the system makes the next statement (step 741) and returns to waiting for a response (step 713).

If the quality of the response was found to be one of non-valid, not understood or not in the system's vocabulary, the system initiates a special script (step 748), such as a loss of understanding/responsiveness query (described further below). The statement that was determined to be non-valid, not understood, delayed or not in the system's vocabulary is repeated (step 752). A response is awaited (step 753). A similar determination as in step 730 is made on the response (step 758). If the system receives a valid response, the system returns to step 732. If the response is not a valid response, the system initiates further verbal interaction (step 760). If the system receives a valid response (step 762), the system returns to step 732. If the system receives a response that is not valid (step 763), such as a non-valid response, a not understood response, a response not using system recognized vocabulary or a delayed response, the system initiates specific checks for emergencies, including a check for a loss of responsiveness (step 764), loss of understanding (step 766) or another possible emergency (step 768). The system can use the data structures described above. The specifics of how the system can make the decisions are also described further below.

In some embodiments, the system being an interactive session with the client after checking to see if the “Start Up IS” Flag is set and finding the flag set. The system then beings executing an IS (i.e., to start up a new conversation with the client). The data that is required is contained in the Active ReIS DS. The OTS is output to the client by carrying out an “Output the OTS” Routine, such as follows.

“Output The OTS” Routine
Get the OTS from the Active ReIS Data Store
Clear out the contents of the NI Register & CIF Flag
If there is an OTS Instruction, execute it
If verbal interaction (VI) is enabled:
   Put the OTS into the OTS-V Register
   Set the OTS-V Flag
If screen/keyboard input (SKI) is enabled:
   Put the OTS into the OTS-SK Register
   Set the OTS-SK Flag
Continue

The system is also continuously checking for an input from the client. When the system has an input, it sets the input text string (ITS) flag, herein the ITS-V-R Flag (for verbal input or the ITS-SK-R Flag for input from a screen/keyboard device, such as a user input device), and puts the input into the ITS-V-R Register (ITS-SK-R Register). When the system finds a set Flag, it grabs the input from the ITS-V-R Register (or ITS-SK-R Register). There are 5 types of inputs that can be received: one of the Valid Inputs, associated with the OTS; “Too Much Time” Code; “Un-recognizable Word(s)” Code; “Non-Understood” Code; “Non-Valid Input” Code.

When the system receives an Input, it then carries out the Decision Statement associated with the currently active IU. The system works with data in the Active ReIS Data Store. The system goes through each of the Conditions in the Decision Statement, looking for a True Condition. There are 3 types of Conditions. A Valid Input Condition is a “Condition” that simply is one of the Valid Inputs associated with the current IU. When the Input received matches one of the Valid Inputs listed in the Decision Statement, then the Valid Input is considered “True”. A Code Condition “Condition” is simply one of the four special Codes. When the Input received matches one of the Codes listed in the Decision Statement, then that Code is considered “True”. A Special Condition refers to a Condition that is a Logic Statement. A Special Condition is usually made up of one or more Valid Inputs plus some other variable. Example: {(“Yes”) AND (Heart Rate>100 per min.)}

When the Logic Statement of a Special Condition is True, then that Special Condition is considered “True”. If no Condition in the Condition List is “True”, the “Universal” Conditions associated with the IS are checked. A “Universal” Condition is one that is associated with every IU in the IS. There are four possible “Universal” Conditions: TMT-IS; URW-IS; NVI-IS; NUI-IS.

An IS is said to have a “Universal” Condition when there is an Action Statement in the “Universal” Condition field of the IS Definition. When the Input received matches one of the “Universal” Conditions, then that “Universal” Condition is considered “True”. If no Conditions are True, then the next IU is executed. When a True Condition is found, it then carries out the Action, or Actions, associated with the True Condition.

There are several different types of Actions:
1) <GOTO IU#xxx>
2) <GOTO IS#yyy/IU#xxx>
3) <CALL IU#xxx>
4) <CALL IS#yyy/IU#xxx>
5) <RETURN>
6) <RETURN-REPEAT>
7) <END SESSION>
8) <SAVE>
9) <SAVE “ttt”>
10) <SAVE Tx>
11) <TSAVE Tx>
12) <TSAVE Valid Inputs>
13) <Cx=Cx+1>
14) <WAIT>
15) <RxSAVE “yyy”>
16) <NSAVE “yyy”>

An action statement can be executed as in the following examples.

1. <GOTO IU#xxx>: Carry out (another) IU
If the Action is a pointer to a IU (in the Active ReIS), then:
   Place the current IU# into the Previous IU Register; place the current Valid Inputs into the Previous Valid Inputs Registers.
   Go to the IS Store, and access the Record of IU#xxx (of the Active ReIS)
   Load the data in the Record into the ReIS DS (of the Active ReIS)
   Carry out the “Output the OTS” Routine.
Wait for the next input (ITS-V-R Flag=1, or ITS-SK-R Flag=1).

2. <GOTO IS#yyy/IU#xxx>: Carry Out Another IU, in a Different IS
If the Action is a pointer to a IU, in an IS other than the Active ReIS, then:
   Place the current IU# into the Previous IU Register; place the current Valid Inputs into the Previous Valid Inputs Registers.
   Go to the IS Store, and access the IS having the IS#yyy
   Get the IS-related data, and the data associated with the IU#xxx, from the IS
   Load this data, plus the IU#, into the Active ReIS DS
   Carry out the “Output the OTS” Routine.
Wait for the next input (ITS-V-R Flag=1, or ITS-SK-R Flag=1).

3. <CALL IU#xxx>
If the Action is a CALL to an IU (in the Active ReIS), then:
   Place the current IU# into the Previous IU Register; place the current Valid Inputs into the Previous Valid Inputs Registers.
   Go to the IS Definition of the presently Active ReIS, and get the IU# of the next IU in sequence.
   Put this IU#, and the IS# of the present IS into the “CALL Return” Register of the Active ReIS DS. (Note: There are four Call Return Registers. Use the Register with the lowest number that is unoccupied.)
   Put the present IU# into the “Previous IU” Register of the Active ReIS DS
   Go to the IS Store, and access the Record of IU#xxx (of the present IS)
   Load the data in the Record into the ReIS DS (of the Active ReIS)
   Carry out the “Output the OTS” Routine.
Wait for the next input (ITS-V-R Flag=1, or ITS-SK-R Flag=1).

4. <CALL IS#zzz/IU#xxx>
If the Action is a CALL to an IU, in an IS other than the Active ReIS, then:
   Place the current IU# into the Previous IU Register; place the current Valid Inputs into the Previous Valid Inputs Registers.
   Go to the IS Definition of the presently Active ReIS, and get the IU# of the next TU in sequence.
   Put this IU#, and the IS# of the present IS, into the “CALL Return” Register of the Active ReIS DS. (Note: There are 4 Call Return Registers. Use the Register with the lowest number that is unoccupied.)
   Put the present IU# into the “Previous IU” Register of the Active ReIS DS
   Go to the IS Store, and access the IS having the IS#zzz
   Get the IS-related data, and the IU#xxx data, associated with IS#zzz
   Load this data, plus the IS#, into the Active ReIS DS
   Carry out the “Output the OTS” Routine.
Wait for the next input (ITS-V-R Flag=1, or ITS-SK-R Flag=1).

5. <RETURN>
If the Action is to RETURN from a CALL, then:
   Find the first occupied “Call Return” Register (in the Active ReIS DS), beginning with #4 and going to #1.
   Get IS# (zzz) and IU# (xxx) from this “CALL Return” Register.
   If the IS# is the same as the present IS#:
      Go to the IS Store, and access the Record of IU#xxx
   If the IS# is not the same as the present IS#:
      Put the IS# into the IS# register of the Active ReIS DS
      Go to the IS Store, and access the Record of IU#xxx (of IS#zzz)
   Load the data in the Record into the ReIS DS (of the Active ReIS)
   Carry out the “Output the OTS” Routine.
Wait for the next input (ITS-V-R Flag=1, or ITS-SK-R Flag=1).

6. <SAVE>
This Action is used to instruct a save of the Valid Input in the Parameter DSA of the IMP whose # is given in the IMP# Column, as well as to save the timestamp.

7. <SAVE “ttt”>
This Action is used to instruct a save of the text “ttt” in the Parameter DSA of the IMP whose # is given in the IMP# Column, as well as to save the time stamp.

8. <SAVE Tx>
This Action is used to instruct a save of the value contained in Temporary Register Tx, in the Active ReIS DS, into the DSA of the IMP listed in the IMP# Column of the IU, as well as to save the time stamp.

9. <TSAVE Tx>
This Action is used to instruct a save of the Valid Input value into Temporary Register Tx, in the Active ReIS DS.

10. <TSAVE Valid Inputs>
This Action is used to instruct a save of the Valid Inputs of the present IU in the Valid Inputs Temporary Store of the ReIS Data Store.

11. <Cx=Cx+1>
This Action is used to instruct an increment to the number in Register, Cx, in the Active ReIS DS.

12. <WAIT-zzzzS IS#yyy> or <WAIT-hh:mm:ss IS#yyy>
This Action is used to instruct activation of IS#yyy in zzzz seconds from now, or at the time of hh:mm:ss. The system loads the Activate Time into the Trigger Condition Description field of the Record associated with IS#yyy (in the PT Table or RT Table).

13. <RETURN-REPEAT>
If the Action is to RETURN-REPEAT from a CALL, then:
   Get IS# (zzz) in the “CALL Return” Register (in the Active ReIS DS).
   Get IU# (xxx) from the “Previous IU” Register
   If the IS# is the same as the present IS#:
      Go to the IS Store, and access the Record of IU#xxx
   If the IS# is not the same as the present IS#:
      Put the IS# into the IS# register of the Active ReIS DS
      Go to the IS Store, and access the Record of IU#xxx (of IS#zzz)
   Load the data in the Record into the ReIS DS (of the Active ReIS)
   Carry out the “Output the OTS” Routine.
The system then waits for the next input (ITS-V-R Flag=1, or ITS-SK-R Flag=1).

14. <END SESSION>
If the Action is to END the IS, then:
   Go to the PT Table and find every PT Record that has an IS# that is the same as the # of the IS that is “ENDing”.
      Set the “Currently Being Addressed” Flag of each of these Records to 0.
      Access the DSA of all the Parameters in the PT Conditions of these Records and save the value, “JFA” (Just Finished Analysing), and the timestamp.
   Do the same to the RT Table.
   Clear out all the fields of the presently Active ReIS.

15. <RxSAVE “yyy”>
If the Action is to <RxSAVE>, then:
   Save “yyy” in Register REGx

16. <NSAVE “yyy”>
If the Action is to <NSAVE>, then:
   Save “yyy” in Register NI

The PT Table, RT Table, CIIC Table, and the Parameter DSA can be used to determine when an IS should be carried out, and which IS should be carried out. Incorporated into this process is the objective of optimizing the frequency of verbal interaction with the client.

The system can go through each of the Trigger Conditions (TC) listed in the PT and RT Tables. It evaluates each TC to see if it is True. If it finds a True Condition, it places the associated IS# in the ReIS Register, and it sets the ReIS Flag. When it finishes evaluating all the Conditions, it starts all over again. This can go on indefinitely.

As all of the Records in the PT Table and RT Table are cycled through, each of the listed Conditions is evaluated. The following process can be carried out:
Get the next Record from the PT Table.
   If the “Currently Being Addressed” Flag=1, of that Record, then get the next Record.
Get the content of the Trigger Condition field
   If it is a Logic Statement, evaluate it
      Access the Parameter Data Storage Areas of the Parameters contained in the Logic Statement.
      Check the next-to-latest values of these Parameters.
         If any of these values is a “JFA” value, then Logic Statement is False. Do not set the Condition Flag.
      Get the latest values of the Parameters
         If the Logic Statement is False, do not set the Condition Flag.
         If the Logic Statement is True, set the Condition Flag
   If it is a CIIC Code (CIIC#xx):
      Check the CIIC Flag associated with the CIIC Code in the CIIC Table
      If the CIIC Flag is set, set the Condition Flag, and clear the CIIC Flag in the CIIC Table
   If it is a Trigger Condition Pointer (TCP#xx):
      Execute the TC Subroutine pointed to by the TC Pointer.
      Access the Parameter Data Storage Areas of the Parameters contained in the TC Subroutine.
      Check the next-to-latest values of these Parameters.
         If any of these values is a “JFA” value, then the TC Subroutine is False. Do not set the Condition Flag
      Get the latest values of the Parameters
         If the TC Subroutine is False, do not set the Condition Flag.
         If the TC Subroutine is True, set the Condition Flag
      The Subroutine then RETURNs.
The system checks the Condition Flag.
   If the Flag is not set:
      Get the next Record from the PT Table; Repeat the above.
   If the Flag is set:
   Set the “Currently Being Addressed” Flag in the Record.
      Check if any other PT Record, with a set “Currently Being Addressed” Flag, has the same associated IS as the present PT Record.
      If No, then a) Put the associated IS#, from the Record, into the ReIS Register, b) Set the ReIS Flag
      If Yes, then do next: Get the next Record from the PT Table; Repeat the above.

When the system goes through every Trigger Condition in the PT Table, it then goes to the RT Table and repeats the above with every Record in the RT Table. When the system finishes with the PT Table, it then repeats the above again.

Together, multiple ReIS Data Stores are used to carry out handling IS Requests, activating another IS if a presently active IS is completed and handling emergency based IS requests. Multiple requested ISs can be handled together to form multiple conversations using the ReIS Data Stores.

When a new IS Request is received (e.g., ReIS Flag is set), the system gets the IS# from the ReIS Register, and then loads the information associated with the new IS into an empty ReIS DS. The following steps can be carried out:
Clear out all the registers associated with the “empty” ReIS DS.
Go to the ISD Store, and access the IS having the above IS#
Get the following data from the IS:
   IS-related data
   Data associated with the first IU, from the IS
Load this data into an empty ReIS DS

Then, how the new IS request is to be handled is decided. There are six possible situations:

a) No presently Active ReIS

b) Presently active ReIS; Priority of New ReIS>Priority of Active ReIS; No ReIS-in-Waiting

c) Presently active ReIS; Priority of New ReIS>Priority of Active ReIS; ReIS-in-Waiting

d) Presently active ReIS; Priority of New ReIS<=Priority of Active ReIS; No ReIS-in-Waiting

e) Presently active ReIS; Priority of New ReIS<=Priority of Active ReIS; ReIS-in-Waiting; Priority of New ReIS>Priority of ReIS-in-Waiting

f) Presently active ReIS; Priority of New ReIS<=Priority of Active ReIS; ReIS-in-Waiting; Priority of New ReIS <=Priority of ReIS-in-Waiting

The following describes how each of these situations can be handled:

a) No presently Active ReIS
Make the New ReIS Active by putting “Active” into the Status field of the New ReIS's Data Store.
Set the “Start Up IS” Flag
Continue

b) Presently active ReIS; Priority of New ReIS >Priority of Active ReIS; No ReIS-in-Waiting
Get the IU Group # associated with the present IU, of the present Active ReIS (found in the ReIS DS).
Go to the IS Store, and obtain the # of the first IU in this IU Group.
Obtain all the data associated with this IU, and put the data into the DS of the presently Active ReIS.
Change the content of the Status field of the present Active ReIS to “2”. This indicates that the ReIS is now an ReIS-in-Waiting.
Put “Y” into the “IS Interrupted” field of the DS associated with this ReIS. This indicates that the ReIS was interrupted, while in progress.
Make the New ReIS active by putting “Active” into the Status field of the New ReIS's Data Store.
Send the following OTS to the OTS-V Register, to be spoken or sent as text to the client: “John, I have to interrupt the present conversation, and start up a new conversation.”
Set the “Start Up IS” Flag
Continue

c) Presently active ReIS; Priority of New ReIS>Priority of Active ReIS; ReIS-in-Waiting

The same activities as in the situation above plus the following:
Change the content of the Status field of the ReIS-in-Waiting to “3”. This makes it an ReIS-in-Waiting-2.

d) Presently active ReIS; Priority of New ReIS<=Priority of Active ReIS; No ReIS-in-Waiting
Put “2” into the Status field of the New ReIS's Data Store. This makes it an ReIS-in-Waiting.

e) Presently active ReIS; Priority of New ReIS<=Priority of Active ReIS; ReIS-in-Waiting; Priority of New ReIS>Priority of ReIS-in-Waiting
Put “3” into the Status field of the DS of the present ReIS-in-Waiting.
   This makes it an ReIS-in-Waiting-2.
Put “2” into the Status field of the New ReIS's Data Store. This makes it an ReIS-in-Waiting.

f) Presently active ReIS; Priority of New ReIS<=Priority of Active ReIS; ReIS-in-Waiting; Priority of New ReIS<=Priority of ReIS-in-Waiting
Put “3” into the Status field of the DS of the new ReIS. This makes it ReIS-in-Waiting-2.

An ReIS-In-Waiting can be activated after an IS has finished. The system continuously checks to see if an active ReIS has just finished. If it has, the system then checks to see if there is an ReIS-in-waiting. If there is one, the following happens:
If the ReIS-in-Waiting was not interrupted:
   Change the content of the Status field of the ReIS-in-Waiting to “Active”.
   If there was a 3rd ReIS, make it the ReIS-in-Waiting (by putting “2” into its Status field).
   Set the “Start Up IS” Flag.
   Continue
If the ReIS-in-Waiting had been interrupted
   The system checks how long it's been since the ReIS-in-Waiting was interrupted.
   If the interruption was not too long {(Present Time—IS Interruption Time)<T-InterruptMax}, then:
      Change the content of the Status field of the ReIS-in-Waiting to “Active”.
      Clear out the IS Interrupt Status field
      If there was a 3rd ReIS, make it the ReIS-in-Waiting (by putting “2” into its Status field).
      Speak out, e.g.,: “John, I now want to continue the conversation that I was having with you a few minutes ago.”
      Set the “Start Up IS” Flag.
      Continue
   If the interruption time was too long, then carry out the interrupted ReIS-in-Waiting from the beginning:
      Obtain all the data associated with IU#1 of the ReIS-in-Waiting, and load the data into its DS.
      Change the content of the Status field of the ReIS-in-Waiting to “Active”.
      If there was a third ReIS, make it the ReIS-in-Waiting (by putting “2” into its Status field).
      Speak out, e.g.,: “John, I now want to continue the conversation that I was having with you a while ago. Because of this lengthen of time, I need to start from the beginning of the conversation.”
      Set the “Start Up IS” Flag.
      Continue

An IS Request can be handled when an Emergency is detected as follows. An ED Flag is set. When this happens, the system immediately makes the Requested IS from the Active ReIS. The following steps are then carried out.
Go to the IS Store, and access the IS having the IS# provided
Get the IS-related data, and the data associated with the first IU, from the IS
Load this data, into an Empty ReIS DS. (If there is no Empty ReIS DS, then overwrite the ReIS-in-Waiting-2 DS.)
Put “Active” into the Status field of the New ReIS's Data Store.
If there is no presently Active ReIS, then:
   Set the “Start Up IS” Flag
If there is a presently Active ReIS, then:
   Make the Active ReIS into ReIS-in-Waiting
   If there was an existing ReIS-in-Waiting, make it ReIS-in-Waiting-2
   Speak the following: “John, I have to interrupt the present conversation.”
   Set the “Start Up IS” Flag

The VV&I Table (Table 53), CIIC Table (Table 54), and the ReIS DS are used to perform functions, such as accepting verbal input from the client, interpreting the input, sending the input for further processing and determining a delay in the client's response.

The system handles the verbal inputs as follows. The system continuously checks for new verbal input from the client. It does this by checking the ITS-V Flag. If the Flag is set, then there is a new input text string (ITS) waiting in the ITS-V Register. In some embodiments, the system works with Input Text Strings, not individual words, unless there is only one word in the client's response. If there is an ITS to be picked up, it takes in the content of the ITS-V Register, and interprets it.

For Unrecognizable Words/Verbal Input, the system checks to see if the ITS contains any unrecognizable words, that is, spoken words that the are not recognized. If unrecognizable words are found, or more specifically, if text code that indicates unrecognizable words is found, the system prepares a special code, e.g., URW Code, that indicates this. It then puts the Code into the ITS-V-R Register, and sets the ITS-V-R Flag.

When the ITS is not a Time Code or unrecognizable ITS, the system then checks to see if the ITS is one of the Valid Inputs associated with the OTS, that is listed in the present IU. This is for a Valid Input/Reply.

First, the system utilizes the VV&I Table to “interpret” the ITS; it looks for a match. If it finds a match, it goes to the Active ReIS Data Store to see if this “interpretation” is one of the Valid Inputs. If it is, the system puts this interpretation into the ITS-V-R Register, and sets the ITS-V-R Flag. It also puts the interpretation into the NI Register.

For example, the system says something to the client that has associated Valid Inputs of: “No”, “Yes”, “Sometimes”. The client responds by saying something that, after conversion, is the following ITS: “Sure, I guess so.” The system utilizes the VV&I Table and finds that one of the interpretations of the words, “Sure, I guess so” is “Yes”. It then checks the Active ReIS DS, and finds that one of the Valid Inputs is “Yes”. Thus, the system has determined that the client has just spoken a Valid Input.

If the system determines that the ITS is not one of the Valid Inputs, it then checks to see if the client was not replying to the OTS, but in fact, was saying something on their own initiative. For example, the client may ask for the present time. This occurs during a Client-Initiated Interaction.

The system checks for CII's by carrying out the following:

Each of the CIIC's in the CIIC Table are evaluated, using the ITS. If True CIIC is found, the corresponding CIIC Flag is set.

The following is also performed:

a) The system checks if there is anything in the IMP Column. If there is, it saves the specified value into the DSA of the IMP whose IMP# is given in the IMP Column. The Timestamp is also saved.

b) The system checks if there is a value in the NI Column. If there is, it saves the value into the NI Register.

c) The system sets the CIF Flag.

The system is then finished with that ITS.

Immediately after this, the system, will find the above set CIIC Flag and handle the CII.

If the ITS was properly interpreted by the VV&I Table (i.e., a match was found), the ITS was not a Valid Input, and was not interpreted by the CIIC Table, then the ITS is considered a Non-Valid Input. The system prepares a special code that indicates that the ITS is a Non-Valid Input (NVI Code), and puts it into the ITS-V-R Register, and sets the ITS-V-R Flag.

If the ITS is not a TMT Code, Unrecognizable Verbal Input, Valid Input, Client-Initiated (Verbal) Interaction Condition, or Non-Valid Input, then the system prepares a special code that indicates that the ITS is not understood, and puts it into the ITS-V-R Register, and sets the ITS-V-R Flag.

As noted herein, the client's response can be delayed. If there is no new ITS, the system checks how long it has been since the latest OTS was sent to the client, with no client response. If it has been too long, the system creates a special code to note this fact. The following describes the process:
Get the value in the “OTS-V Done” Register, in the Active ReIS DS.
Get the RDM Value from the RDM-IU Register in the Active ReIS DS. If there is no value in this Register, get the RDM Value from the RDM-IS Register in the Active ReIS DS.
Is {(Present Time−“OTS-V Done” Time)>RDM Value}?
   If No
      Repeat cycle
   If Yes
      Put “Too Much Time” (TMT) Code into the ITS-V-R Register
   Set the ITS-V-R Flag=1
   Repeat cycle

This sequence can be performed many times a second.

One of the purposes of the interaction with the client is to get values for Interaction-Monitored Parameters (IMP), and to save these values in the DSA. IMP handling is carried out during a <SAVE> Action, while an Interaction Session is executing. When an IU is directly associated with an IMP, the IMP# is included in the IU Record. When the client responds to the OTS of such an IU, and the response is a Valid Input, the this Input is saved in the DSA of the IMP, along with timestamp information.

The following illustrates how this is carried out:

In Table 16 is a portion of an IS. If the client responded with “Yes” to IU#20, IU#40 will execute. If one of the Valid Inputs from the client is received, which are also valid values associated with IMP#xx, the Action associated with the Input is carried out. If the client replied with “Mild”, the Action associated with “Mild” is “<SAVE>∥<IU#50>”.

The following is carried out:
The # of the IMP associated with this Input (in this case: xx) is obtained from the IMP# Column.
The DSA of this IMP is accessed.
The value “Mild” is saved in the DSA, as well as a timestamp.
The IS continues, by going to IU#50 and executing the IU.

TABLE 16

| | | | | | | RMD- |
|---|---|---|---|---|---|---|
| IU | Output | Decision Statement | | IU | | IU |
| # | Text String | Condition | Action | Group | IMP# | (secs) |
| 10 | | | | | | |
| 20 | Do you have | Yes | <GOTO | | 1 | |
| | sudden | | IU#40> | | | |
| | numbness | No | <GOTO | | | |
| | or weakness | | IU#75> | | | |
| | on one side | | | | | |
| | of your | | | | | |
| | body? | | | | | |
| 40 | Is it mild or | Mild | <SAVE>\|\|<IU# | | | Xx |
| | serious? | | 50> | | | |
| | | Serious | <SAVE>\|\|<IU# | | | xx |
| | | | 50> | | | |
| 50 | | | | | | |

Non-verbal input entered by the client into the system can be continuously monitored. The system does this by checking the ITS-SK Flag. If the Flag is set, then there is a new input text string (ITS) waiting in the ITS-SK Register. If there is an ITS to be picked up, it takes in the content of the ITS-SK Register. The input will have the format: “Xn”, where “X” is a letter and “n” is a number up to 10,000. If the letter is a “V”, then the following number represents the selection of the nth Valid Input. If the letter is a “C”, then the client has selected one of the Client Initiated Interaction (CII) Conditions.

If the ITS is “Vn”, the system goes to the Active ReIS DS, and gets the Valid Input associated with this number. The system puts it into the ITS-SK-R Register, and sets the ITS-SK-R Flag. If the ITS is “Cn”, indicating client initiated interaction, the system accesses the CIIC Table and sets the CIIC Flag associated with the CIIC that has that number.

As with monitoring verbal responses for delay, the system can also monitor the non-verbal input. If there is no new ITS, the system checks how long it has been since the latest OTS was sent to the client, with no client response. The following describes the process:
Get the value in the “OTS-SK Done” Register, in the Active ReIS DS.
Get the RDM Value from the RDM-IU Register in the Active ReIS DS. If there is no value in this Register, get the RDM Value from the RDM-IS Register in the Active ReIS DS.
{(Present Time−“OTS-SK Done” Time)>RDM Value}?
   If No
      Repeat cycle
   If Yes
      Put “Too Much Time” (TMT) Code into the ITS-SK-R Register
      Set the ITS-SK-R Flag=1
      Repeat cycle

The cycle is performed many times a second.

Early warning signs of an SHE, or the early stage of an SHE and serious safety situations may be detected using Emergency Detection (ED) Conditions, the ED Table, and the Parameter DSA.

An ED Condition is a Logic Statement that specifies a situation that is considered to be an Emergency situation. Each ED Condition consisting of:
One or more parameters (PP, IMP, SMP, VMP)
Specific values
Logical operators (e.g., AND, OR)

An example of an ED Condition is: {(Heart Rate<20/minute for 1 minute) AND (No Response from client)}. Detection of this ED Condition may indicate cardiac arrest. The ED Table contains a list of every ED Condition that is recognized. The follow can be performed to determine an emergency situation.

All the records in the ED Table are cycled through on an ongoing basis, where each of the ED Conditions listed is evaluated. When a live situation occurs that presents parameters values that make one of the Conditions “True”, then the system interprets this as an Emergency Situation.

The system cycles through all the records in the ED Table, evaluating each of the Emergency Detection (ED) Conditions listed. The following process is carried out:
Get the next Record from the ED Table.
Get the content of the Trigger Condition field.
   If it is a Logic Statement, evaluate it.
      Access the values of Parameters in the Parameter DSA, as required.
   If the Logic Statement is True, set the Condition Flag.
   If it is a Trigger Condition Pointer (TCP#xx):
      Execute the TC Subroutine pointed to by the TC Pointer.
      If the Condition is TRUE, it sets the Condition Flag.
      The Subroutine then RETURNS.
The Condition Flag is checked.
   If the Flag is not set:
      Get the next Record from the ED Table; Repeat the above.
   If the Flag is set:
      Set the ED Flag.
      Put the associated EDIS#, from the Record, into the EDIS# Register.

An EDIS, or Emergency Detection Interaction Session, is an IS that is carried out when an Emergency is detected. Purposes of the EDIS include, informing the person that an Emergency has been detected and that the ERD is being notified, informing the person what type of Emergency it is, giving instructions to the person, e.g., please sit down, beside the telephone, and trying to re-assure the person.

When the system determines that an emergency is occurring, the following can take place. An ED Flag is set. A client record is obtained from a database containing the client records. Additional information can be sent to the emergency services or control center., such as caller ID information. An Emergency Summary Report of the emergency situation can be compiled and sent to the emergency service or control center. This Emergency Summary Report can include one or more of the following:
The potential problem
How/why the decision was made, and the relevant data
   The Emergency Trigger that was activated
   The Parameters, and their values, that activated the EA
The present state of the person
   The values of all the Parameters for the past hour
   A summary of all the Parameters for the last 24 hours
The person's vital signs measurements, in real time optional

This information can also be saved in the client information database and can be used to help the Emergency Response personnel to better evaluate the situation.

The following is a list of algorithms and processes that can be used to create the data described above, that is, the data in the data tables and ISD store is derived from these algorithms and processes. First, the algorithms used for detecting key SHEs are described. Then the processes, or steps, used for detecting SHEs are described. Finally, the actual functionality data, the data that is loaded into the ISD Store, the PT Table, and the ED Table, is described.

The following lists the SHEs that the system monitors for and detects: stroke and transient ischemic attack, heart attack and unstable angina, cardiac arrest, unconsciousness, loss of understanding/incoherence/confusion, loss of responsiveness, a bad fall, severe pain /illness/weakness, can't move/can't walk, severe breathing problem, a general SHE.

Stroke is difficult to detect with personal health monitoring devices. The early warning signs and the occurrence of stroke, however, may be detected through verbal and visual means. The American Stroke Association says that these are the warning signs of stroke:
Sudden numbness or weakness of the face, arm or leg, especially on one side of the body
Sudden confusion, trouble speaking or understanding
Sudden trouble seeing in one or both eyes
Sudden trouble walking, dizziness, loss of balance or coordination
Sudden, severe headache with no known cause

In addition there are two well-known Checklists that are used by many emergency response personnel across North America to assist in determining to a high probability if a person is experiencing a stroke. These Checklists are called: Los Angeles PreHospital Stroke Screen (LAPSS), and Cincinnati PreHospital Stroke Scale. The following lists the key elements of each Checklist.

Los Angeles PreHospital Stroke Screen:
Facial smile/grimace: Right side droop, or left side droop
Grip: Weak or no grip with left hand or right hand; not both
Arm weakness: When both arms held out at same time, one arm drifts down, or falls rapidly, compared to the other one; not both

Cincinnati PreHospital Stroke Scale:
Facial Droop: One side of face does not move at all
Arm Drift: One arm drifts compared to the other
Speech: Slurred or inappropriate words or mute

The system utilizes the following Logic Statement in its process to monitor for, and detect, Stroke. This Statement is derived from the above definition of a Stroke.

{((Sudden numbness/weakness in one arm, one leg, or one side of the face) [1]
OR

(Positive Arm Drift Test)) [2]
AND

((Trouble speaking) [3]
OR

(Confused) [4]
OR

(Mute) [5]

OR

(Problem smiling) [6]

OR

(Droopy face—on one side))} [7]

The following explains how each of the Conditions is evaluated:

[1]: This information is obtained, such as by verbal interaction with the client. Or the client may verbally give this information directly to the system, such as after a self-initiated test.

[2]: The system, or emergency personnel asks the client to stand in front of the video monitor; hold arms straight out in from of him/her. If one arm drifts down, or falls, much differently than the other arm, then this is a “True” test result. Special image recognition software determines a result for this Test. Alternatively, if the client is able to self evaluate, the Service can ask the client to do the above test and input the results. The client then speaks the result to the system or emergency personnel.

[3]: Using CHVI with the individual, the system asks the person to say certain words and checks that person speaks alright, or has difficulties speaking. In addition, the person is continuously monitored for problems speaking.

[4]: The person is asked a question that requires a certain answer that he/she knows. Whether the person has problems answering properly is determined. In addition, the system, or emergency personnel, continuously monitors if the person appears confused.

[5]: The person is asked a question. The system checks for no verbal response. In addition, the system continuously monitors for no verbal response from the person.

[6], [7]: The client is asked to stand in front of the video monitor, very close. Special image recognition software determines if the person's face is droopy on one side (or if the person can smile or not). Alternatively (if the client is able to) the Service can ask the client to get up close to a mirror and to check their face for droopiness on one side (or whether the person can smile or not). The client then speaks the result to the system.

Most heart attacks start slowly, with mild pain or discomfort. Often people affected aren't sure what's wrong and wait too long before getting help. Heart attacks are difficult to detect with personal health monitoring devices. The early warning signs, and the occurrence, of a heart attack may be detected through verbal and visual means.

The American Heart Association indicates that the following signs can mean a heart attack is happening:
Chest pain/discomfort in the center of the chest; lasts for more than 5 minutes, or goes away and comes back
   Uncomfortable pressure; Severe pressure; Squeezing; Fullness
Pain/discomfort in one or both arms, the back, neck, jaw or stomach.
   May or may not spread from the center of the chest
Other symptoms:
   Shortness of breath; Nausea; Dizziness; Lightheadedness; Cold sweat

The system utilizes the following logic statement in its process to monitor for and detect a heart attack. This statement is derived from the above definition of a heart attack.

{((Pain in the center of the chest; Lasts for more than 5 minutes) [1]
OR

((Pain in the center of the chest; Starts—Goes away—Comes back for more than a few minutes) [2]
OR

(Discomfort in the center of the chest—Pressure, Fullness, or Squeezing; Lasts more than 5 minutes)) [3]
OR

(Discomfort in the center of the chest—Pressure, Fullness, or Squeezing; Starts—Goes away—Comes back for more than a few minutes))} [4]

[1], [2], [3], [4]: This information is obtained by verbal interaction with the client. Or the client may verbally give this information directly to the Service.

The above list of heart attack-related algorithms is related to one implementation of the system. Other implementations of the system could use modified versions of these algorithms, different algorithms, other algorithms or different numbers of algorithms.

In addition to heart attack, the system can monitor and detect the early warning signs before a cardiac arrest occurs or the occurrence of cardiac arrest, such as by using a one or a combination of monitoring devices, verbal interaction and visual and audio means. The American Heart Association says that the signs of cardiac arrest are:
Sudden loss of responsiveness. No response to gentle shaking.
No normal breathing. The victim does not take a normal breath when you check for several seconds.
No signs of circulation. No movement or coughing.

The system utilizes the following two logic statements in its process to monitor for, and detect, the early warning signs of cardiac arrest, and the occurrence of cardiac arrest. These Statements are derived from the above definition of cardiac arrest.

Possible EWSs of Cardiac Arrest

{((Heart Rate low) [1]
OR

(Blood Pressure low) [2]
OR

(ECG signal not normal) [3]
OR

(BOS low)) [4]
AND

((Client says that feels Bad) [5]
OR

(Client provides no verbal response) [6]
OR

(Client shows signs of confusion/use of inappropriate words) [7]
OR
(Client says Emergency)} [8]

[1]: This information is obtained from either the ECG Monitor, Pulse Oximeter, or Heart Rate Monitor.

[2]: This information is obtained from the Blood Pressure Monitor.

[3]: This information is obtained from the ECG Monitor.

[4]: Information obtained from the Pulse Oximeter.

[5], [6], [7], [8]: This information is obtained through CHVI.

Indicia of an occurrence of cardiac arrest

{((Heart Rate low) [1]
OR

(Blood Pressure low) [2]
OR

(ECG signal bad) [3]
OR

(BOS low)) [4]
AND

((Client is unconscious) [5]
OR

(Clients has Loss of Response)} [6]

[1]: This information is obtained from either the ECG Monitor, Pulse Oximeter, or Heart Rate Monitor.

[2]: This information is obtained from the Blood Pressure Monitor.

[3]: This information is obtained from an ECG Monitor.

[4]: Information obtained from the Pulse Oximeter.

[5], [6]: This information is obtained through CHVI.

The system monitors for, and detects, falls. When a fall is detected, or there is indication of a possible fall, the system then evaluates the situation to determine if it is an SHE. An SHE may be indicated by a situation where the person is hurt, to the point that he/she cannot move to reach a telephone to call for help or a situation where the person says that the situation is an Emergency, and to please call for help.

The following conditions can indicate a fall.

{((Client says that he/she has fallen} [1]
OR

(Client indicates that he/she has fallen—Vocal sounds, making noise, waving) [2]
OR

(Fall Detection Monitor has detected a fall) [3]
OR

(Sound of falling detected) [4]
OR

(Image of client falling detected)) [5]
AND

((Client says that can't move) [6]
OR

(Client says that it is an Emergency) [7]
OR

(Client non-verbally indicates that it is an Emergency)) [8]
OR

(No verbal response from client)} [9]

[1], [6], [7], [9]: This information is obtained by verbal interaction with the client or the client may verbally give this information directly, self-initiated.

[2]: Obtained by verbally asking the client to respond by making a particular sound, also utilizes the sound recognition capabilities to detect the sounds.

[3]: Obtained by the Fall Detection Monitor. [4], [8]: Obtained by the Sound Recognition module. [5]: Obtained by the Video Monitor and Video/Image Recognition module.

Unconsciousness is an emergency situation because the underlying problem that contributed to the loss of consciousness may be causing other detrimental health problems to the person. Also, the person cannot call for help. Without timely help, the situation could get much worse. The system detects these situations and auto-alerts people who can help. Unconsciousness can be defined as loss of responsiveness and/or no movement. Further, loss of responsiveness refers to no verbal response to a query, no vocal sound to respond to a query, no “noise making” (e.g., knocking on a wall) to respond to a query, and no motion (e.g., waving) to respond to a query.

The system utilizes one or more of the following logic statement to define “unconsciousness”:

{((No verbal response to a query) [1]
AND

(No vocal sound to respond to a query) [2]
AND

(No “noise making” to respond to a query) [3]
AND

(No motion (e.g., waving) to respond to a query)) [4]
AND

((No movement)) [5]
AND

((No client initiated words)) [6]
AND

((Physiological Parameters normal) [7]
OR

(Physiological Parameters—NIL))} [8]

[1]: In the process of verbally interacting with the client, the system records every time that the client does not respond to a query, or, more specifically, when the client takes too long to reply to a query; the TMT Code is utilized for this. If the person does not respond three times in a short period of time, he/she is considered to be in a “No Verbal Response” state. In addition, an IS could test the client for verbal response by asking a question a few times.

[2]: When “No Verbal Response” is detected in a person, the system asks the person to make a vocal sound twice, e.g., a yelp. If no such response is received, a No “Vocal Communications Sound” Response is recorded.

[3]: If a No “Vocal Communications Sound” Response is detected in a person, the system asks the person to make a knocking sound on a nearby surface, twice. If no such response is received, a No “Knocking Communications Sound” Response is recorded.

[4]: If a No “Knocking Communications Sound” Response” is detected in a person, the system asks the person to make a motion, such as waving or lifting a leg, twice. If no such response is received, a No “Motion Communications” Response is recorded.

[5]: Movement, or lack of movement, of the person is monitored by the Video Monitor. If the person is in the view of the Video Monitor, then a value for the “Movement” parameter will be recorded.

[6]: If client is says words, then he/she is not unconscious (by definition).

[7]: If measured physiological parameters are not normal, then the situation may be cardiac arrest as opposed to unconsciousness.

[8]: This means that no physiological parameters are being monitored.

When trying to detect unconsciousness, remotely, it may be a challenge to distinguish it from sleeping. The system can distinguish by using its sound recognition and verbal interaction capabilities. That is, it can listen to the person to check for snoring. In addition, it can detect if the person is lying down or in bed and ask if the person is going to sleep. The system may also sound, similar to an alarm clock, to attempt to wake the client and determine that he is not sleeping. In some embodiments, the system can vibrate a pressure-sensitive mat to attempt to rouse the client. In some embodiments, the system flickers the room lights, such as by sending a signal to a control that communicates with the client's home lighting system, such as through a communications protocol, for example X10. In some embodiments, the system blares a tone and then listens for a response from the client.

With all its capability, the system can determine to a significant degree of accuracy whether or not a person is unconscious. It can then quickly alert emergency response personnel to this fact, and inform them that the person is unconscious (or shows all the signs of unconsciousness.

Loss of responsiveness can refer to no verbal response to a query, no vocal sound to respond to a query, no “noise making” (e.g., knocking on a wall) to respond to a query, no motion (e.g., waving) to respond to a query. It may be important that the situation is quickly evaluated to determine whether it is a serous situation or not.

The system can utilize the following Logic Statement to determine “Loss of Responsiveness”:

{((No verbal response to a query) [1]
AND

(No vocal sound to respond to a query) [2]
AND

(No “noise making” to respond to a query) [3]
AND

(No motion (e.g., waving) to respond to a query) [4]
AND

(NOT[No movement]))} [5]

[1]: In the process of verbally interacting with the client, the system records every time that the client does not respond to a query or, more specifically, when the client takes too long to reply to a query; TMT Code is utilized for this. If the person does not respond three times in a short period of time, he/she is considered to be in a “No Verbal Response” state. In addition, an IS could “test” the client for verbal response by asking a question a few times.

[2]: When “No Verbal Response” is detected in a person, the system asks the person to make a vocal sound twice, e.g., a yelp. If no such response is received, a No “Vocal Communications Sound” Response is recorded.

[3]: If a No “Vocal Communications Sound” Response is detected in a person, the system asks the person to make a knocking sound on a nearby surface, twice. If no such response is received, a No “Knocking Communications Sound” Response is recorded.

[4]: If a No “Knocking Communications Sound” Response is detected in a person, the system asks the person to make a motion, such as waving or lifting a leg, twice. If no such response is received, a No “Motion Communications” Response is recorded.

[5]: Movement, or no movement, of the person is monitored by the Video Monitor. If the person is in the view of the Video Monitor, then a value for the “Movement” parameter will be recorded [Y or N].

The system may test a client for loss of responsiveness by attempting to communicate with the client multiple times, such as three, four or five times prior to contacting emergency services.

A situation may occur when a person being monitored suddenly appears to have lost the ability to understand. The person says words that are inappropriate to the question, or inappropriate to the situation. Loss of understanding also includes confusion, being incoherent, or use of inappropriate words. It can also include sudden loss of mental capacity.

It is a very serious situation because the person is not able to comprehend that they are experiencing a health problem, and that they should be calling for help. Without timely help, the situation could get much worse. It is important that the situation is quickly evaluated to determine whether this is an SHE or not.

The system can detect sudden loss of understanding in two ways:

1. It records every time that a client has given an inappropriate response to a question. This is done by recording the number of NVI Codes and NUI Codes that are generated during an Interaction Session. If the count is significant, in a relatively short period of time, then the system “senses” that the person is showing signs of loss of understanding.

2. The system can also “test” the person for loss of understanding. This is done by asking the person a few basic questions, such as:

a. What day of the week is it?

b. What is your daughter's name?

It can then quickly alert emergency response personnel to this fact, and inform them that the person has loss of understanding.

The following is the ED Condition that is used to detect this SHE:

{((Significant number of improper verbal responses in a short period of time, including emotional outbursts for no reason) [1]
AND

((Client does not pass the “Understanding” Test))} [2]

[1]: This information is gathered by the CHVI, in the process of normal verbal interaction.

[2]: This test is carried out by the CHVI.

A situation when a person suddenly can't walk, or can't move, is an SHE. Since they can't walk, they can't get to the telephone in order to call for help. As they remain in this situation, their condition may get worse.

The ED Condition that is used by the system is:

{((Client says that he/she can't move/walk)
OR

(Client indicates, non-verbally, that he/she can't move/walk))
AND

((Client says that it is an Emergency)
OR

(Client non-verbally indicates that it is an Emergency))}

This ED Condition is contained in the ED Table.

The system monitors for, and detects, SHEs associated with severe pain, illness, and weakness. Specifically, the system monitors for situations where the person is in severe pain/illness/weakness, to the point that they cannot move to reach a telephone to call for help, a situation where the person is in severe pain/illness/weakness, and says that the situation is an emergency.

A possible ED Condition that is used by the system is:

{((Client says “Bad Pain”)
OR

(Client says “Severe Illness”)
OR

(Client says “Severe Weakness”))
AND

(Client says that can't move/walk))}

This ED Condition is contained in the ED Table.

The conditions described above can be used in combination with the method for detecting an emergency to monitor the client. The system monitors the client, such as on a routine basis. The monitoring can include monitoring the client's physical parameters, verbal interaction monitored parameters, sound monitored parameters, and video parameters. The routine verbal monitoring may result in the following conversation taking place between the client and the system. The system asks the client how he/she is doing. If the client says, “Not good”, the system then asks what the problem is. It can then go to a new IS, in this case a master probing IS to collect more information. If the client says, “Good”, the IS may include going through a quick health checklist. If a potential problem is identified while the checklist is being reviewed, the master probing IS takes priority. If everything is fine, the routine IS ends.

A routine IS, IS#R-1, is shown in Tables 17 and 18. Table 17 describes attributes of the ISD at the IS level. An ISD contains an IS record (Table 17) and one or more IU records (Table 18). The TMT-IS, URW-IS, NVI-IS, NUI-IS actions in the IS record may contain an IS to execute if any of these response triggers are detected in any of the IUs being executed. Each IU can have its own response action block as the IS and that if a response action is not available in the executing IU, then the response action in the IS record (if any) will be executed.

TABLE 17

| | | | |
|---|---|---|---|
| IS# | R-1 | TMT-IS Action | <CALL IS#LOS-1/ |
| | | | IU#600> |
| T-InterruptionMax | 180 | URW-IS Action | <CALL IS#LOS-1/ |
| | | | IU#700> |
| RMD-IS | 60 | NVI-IS Action | <CALL IS#LOS-1/ |
| | | | IU#800> |
| S-Time | | NUI-IS Action | <CALL IS#LOS-1/ |
| | | | IU#800> |

TABLE 18

| | | | | | | RMD- |
|---|---|---|---|---|---|---|
| | | Decision Statement | | IU | | IU |
| IU # | Output Text String | Condition | Action | Grp | IMP# | (secs) |
| 10 | John, I want to do a | Good | 200 | | | |
| | quick health check | Not Good | 50 | | | |
| | up on you. | | | | | |
| | But first, how are | | | | | |
| | you feeling? | | | | | |
| 50 | What is the | Pain | <S “Y”> | | PA | |
| | problem? | Illness | <S “Y”> | | IL | |
| | | Weak | <S “Y”> | | WE | |
| | | Numbness | <S “Y”> | | NU | |
| | | Discomfort | <S “Y”> | | DI | |
| | | Breathing | <S “Y”> | | BR1 | |
| | | Fell | <S “Y”> | | FA | |
| | | Trouble Walking | <S “Y”> | | TW1 | |
| | | Chest | <S “Y”> | | CH | |
| | | Heart | <S “Y”> | | HE | |
| | | Can't Move | <S “Y”> | | CM1 | |
| | | Can't walk | <S “Y”> | | CM2 | |
| | | Feel Strange | <S “Y”> | | FS1 | |
| | | Feel Funny | <S “Y”> | | FS2 | |
| | | Something Wrong | <S “Y”> | | FS3 | |
| | | Don't Feel Right | <S “Y”> | | FS4 | |
| | | Nauseous | <S “Y”> | | NAU | |
| | | Dizzy | <S “Y”> | | DIZ | |
| | | Lightheaded | <S “Y”> | | LH | |
| | | Cold Sweat | <S “Y”> | | CS | |
| | | Droopy Face | <S “Y”> | | DF1 | |
| | | Droopy Mouth | <S “Y”> | | DF2 | |
| | | Headache | <S “Y”> | | PA | |
| | | <Other> | 200 | | | |
| 60 | <COMMENT: If | | <R1SAVE | | M1DO | |
| | the person says any | | “1”> \|\| <S | | | |
| | one of the above, | | “Y”> \|\| | | | |
| | control goes to IS# | | <END> | | | |
| | M-1 for health | | | | | |
| | situation analysis. | | | | | |
| | If not, then the | | | | | |
| | person is asked the | | | | | |
| | Quick Checklist.> \|\| | | | | | |
| | <NO OTS> | | | | | |
| 200 | <COMMENT: If | | C7=0 \|\| 210 | | | |
| | the person says any | | | | | |
| | one of the above, | | | | | |
| | control goes to IS# | | | | | |
| | M-1 for health | | | | | |
| | situation analysis. | | | | | |
| | If not, then the | | | | | |
| | person is asked the | | | | | |
| | Quick Checklist.> \|\| | | | | | |
| | <NO OTS> | | | | | |
| 210 | OK, I want to ask | No | <S> \|\| 235 | | PA | |
| | you a few general | Yes | <S> \|\| C7=1 \|\| | | PA | |
| | health questions. | | 235 | | | |
| | After I say a health | | | | | |
| | condition, please | | | | | |
| | reply with: “No or | | | | | |
| | Yes”. | | | | | |
| 235 | Question 1: Any | No | <S> \|\| 240 | | IL | |
| | sudden pain? | Yes | <S> \|\| C7=1 \|\| | | IL | |
| | | | 240 | | | |
| 240 | Any sudden illness? | No | <S> \|\| 245 | | WE | |
| | | Yes | <S> \|\| C7=1 \|\| | | WE | |
| | | | 245 | | | |
| 245 | Any sudden | No | <S> \|\| 250 | | NU | |
| | weakness? | Yes | <S> \|\| C7=1 \|\| | | NU | |
| | | | 250 | | | |
| 250 | Any sudden | No | <S> \|\| 255 | | D1 | |
| | numbness? | Yes | <S> \|\| C7=1 \|\| | | D1 | |
| | | | 255 | | | |
| 255 | Any sudden | No | <S> \|\| 260 | | BR1 | |
| | discomfort? | Yes | <S> \|\| C7=1 \|\| | | BR1 | |
| | | | 260 | | | |
| 260 | Sudden breathing | No | <S> \|\| 265 | | LBA | |
| | problem? | Yes | <S> \|\| C7=1 \|\| | | LBA | |
| | | | 265 | | | |
| 265 | Sudden trouble | No | <S> \|\| 270 | | LCO | |
| | with balance? | Yes | <S> \|\| C7=1 \|\| | | LCO | |
| | | | 270 | | | |
| 270 | Sudden trouble | No | <S> \|\| 275 | | EP | |
| | with coordination? | Yes | <S> \|\| C7=1 \|\| | | EP | |
| | | | 275 | | | |
| 275 | Sudden trouble | No | <S> \|\| 280 | | FS1 | |
| | with eyesight? | Yes | <S> \|\| C7=1 \|\| | | FS1 | |
| | | | 280 | | | |
| 280 | Anything that feels | No | <S> \|\| 281 | | NAU | |
| | “strange”? | Yes | <S> \|\| C7=1 \|\| | | NAU | |
| | | | 281 | | | |
| 281 | Do you suddenly | No | <S> \|\| 282 | | DIZ | |
| | have nausea? | Yes | <S> \|\| C7=1 \|\| | | DIZ | |
| | | | 282 | | | |
| 282 | Sudden dizziness? | No | <S> \|\| 283 | | LH | |
| | | Yes | <S> \|\| C7=1 \|\| | | LH | |
| | | | 283 | | | |
| 283 | Suddenly | No | <S> \|\| 284 | | CS | |
| | lightheaded? | Yes | <S> \|\| C7=1 \|\| | | CS | |
| | | | 284 | | | |
| 284 | Sudden cold sweat? | No | <S> \|\| 290 | | DF1 | |
| | | Yes | <S> \|\| C7=1 \|\| | | DF1 | |
| | | | 290 | | | |
| 290 | <COMMENT: If | C7=1 | <R1SAVE | | M1DO | |
| | the person says Yes | | “2”> \|\| <S | | | |
| | to one or more of | | “Y”> \|\| | | | |
| | the above, control | | <END> | | | |
| | goes to IS# M-1 for | | | | | |
| | health situation | | | | | |
| | analysis. | | | | | |
| | If not, then done for | | | | | |
| | now.> \|\| | | | | | |
| | <NO OTS> | | | | | |
| 295 | <NRR> | | <END> | | | |
| | OK, that's all for | | | | | |
| | now. Everything | | | | | |
| | seems fine. | | | | | |

Table 19 shows yet another exemplary routine table.

TABLE 19

| | | | | | “Currently |
|---|---|---|---|---|---|
| | | | | Interaction | Being |
| | RT | RT Condition | | Session | Addressed” |
| RTC | Priority | Description | RT Condition | (IS) # | Flag |
| RC1 | R5 | Start up a Routine | {Time = 11:00 | R-1 | |
| | | Check IS at 11:00 AM. | AM} | | |
| RC2 | R2 | Start up Routine Check | {Time = | R-1 | |
| | | #1 IS at a random time. | hh:mm:ss} | | |
| RC3 | R3 | Start up the Routine | {Time Since Last | R-1 | |
| | | Check IS if have not | Verbal Statement | | |
| | | heard a verbal | > 2 Hours} | | |
| | | statement from the | | | |
| | | client in over 2 hours. | | | |
| RC4 | R3 | Start up the Routine | {Time Since Last | R-1 | |
| | | Check-in IS if the last | Check-in > 4 | | |
| | | Check-in happened | Hours} | | |
| | | more than 4 hours ago. | | | |
| T1 | R9 | Client wants to know | CIIC# TIM | TIM | |
| | | the present time. | | | |
| TEL1 | R9 | Client wants to know | CIIC# TEL | TEL | |
| | | the telephone number | | | |
| | | for a person or | | | |
| | | organization. | | | |

Table 19

When the routine IS or another monitoring parameter indicates that a trigger has been received or detected, the system goes into probing mode, initiating a probing IS. The master probing IS is referred to as a M-1, and is described further in Tables 20 and 21.

TABLE 20

| | | | |
|---|---|---|---|
| IS# | M-1 | TMT-IS Action | <CALL IS#LOS-1/ |
| | | | IU#600> |
| T-InterruptionMax | 420 | URW-IS Action | <CALL IS#LOS-1/ |
| | | | IU#700> |
| RMD-IS | 60 | NVI-IS Action | <CALL IS#LOS-1/ |
| | | | IU#800> |
| S-Time | | NUI-IS Action | <CALL IS#LOS-1/ |
| | | | IU#800> |

TABLE 21

| | | | | | | RMD- |
|---|---|---|---|---|---|---|
| | | Decision Statement | | IU | | IU |
| IU# | Output Text String | Condition | Action | Grp | IMP# | (secs) |
| 5 | <COMMENT: If the | {(CIF=T) AND | 15 | | | |
| | person just said | ((I#EM1=Y) | | | | |
| | “Emergency” or | OR | | | | |
| | “Help”, ask what the | (I#EM2=Y))} | | | | |
| | problem is.> \|\| <NO | | | | | |
| | OTS> | | | | | |
| 6 | <COMMENT: | {(CIF=T) AND | 700 | | | |
| | Person just said | (I#EMN=Y)} | | | | |
| | “Emergency | | | | | |
| | Now.”> \|\| <NO | | | | | |
| | OTS> | | | | | |
| 7 | <COMMENT: This | CIF=T | 20 | | | |
| | checks if the client | | | | | |
| | just said a health | | | | | |
| | related problem, on | | | | | |
| | their own | | | | | |
| | initiative.> \|\| <NO | | | | | |
| | OTS> | | | | | |
| 8 | <COMMENT: This | REG1=1 | 20 | | | |
| | checks if came here | | | | | |
| | from IS#R-1, or | | | | | |
| | IS#M-2, after the | | | | | |
| | person had indicated | | | | | |
| | a specific problem. | | | | | |
| | If yes, then go to the | | | | | |
| | section beginning at | | | | | |
| | 20.> \|\| | | | | | |
| | <NO OTS> | | | | | |
| 9 | <COMMENT: This | REG1=2 | 570 | | | |
| | checks if came here | | | | | |
| | from IS#R-1, after a | | | | | |
| | Quick Checklist. | | | | | |
| | If yes, then go to the | | | | | |
| | section beginning at | | | | | |
| | 570.> \|\| | | | | | |
| | <NO OTS> | | | | | |
| 10 | <COMMENT: This | I#PP=Y | 900 | | | |
| | checks if came from | | | | | |
| | IS#MPP-1. | | | | | |
| | If yes, go to General | | | | | |
| | SHE Checking | | | | | |
| | section.> \|\| <NO | | | | | |
| | OTS> | | | | | |
| 11 | <COMMENT: This | I#SMP=Y | 12 | | | |
| | checks if came from | I#SMP<>Y | 13 | | | |
| | IS# MS-1. | | | | | |
| | If yes, go to General | | | | | |
| | SHE Checking | | | | | |
| | section.> \|\| <NO | | | | | |
| | OTS> | | | | | |
| 12 | <NO OTS> | S#PSVY=Y | <S “Y”> \|\| 20 | | PA | |
| | | S#FSVY=Y | <S “Y”> \|\| 20 | | FA | |
| | | <Other> | 15 | | | |
| 13 | <COMMENT: This | I#VMP=Y | 14 | | | |
| | checks if came from | I#VMP<>Y | 20 | | | |
| | IS# MV-1. | | | | | |
| | If yes, go to General | | | | | |
| | SHE Checking | | | | | |
| | section.> \|\| <NO | | | | | |
| | OTS> | | | | | |
| 14 | <NO OTS> | V#FSVY=Y | <CALL | | | |
| | | | IS#FA-1> | | | |
| | | TW1=Y | 21 | | | |
| | | V#DF1V=Y | <CALL IS#S- | | | |
| | | | 1> | | | |
| | | V#DF2V=Y | <CALL IS#S- | | | |
| | | | 1> | | | |
| | | <Other> | 500 | | | |
| 15 | What is the | Pain | <S “Y”> | | PA | |
| | problem? | Illness | <S “Y”> | | IL | |
| | | Weak | <S “Y”> | | WE | |
| | | Numbness | <S “Y”> | | NU | |
| | | Discomfort | <S “Y”> | | DI | |
| | | Breathing | <S “Y”> | | BR1 | |
| | | Fell | <S “Y”> | | FA | |
| | | Trouble | <S “Y”> | | TW1 | |
| | | Walking | | | | |
| | | Loss of Balance | <S”Y”> | | LBA | |
| | | Loss of | <S “Y”> | | LCO | |
| | | Coordination | | | | |
| | | Chest | <S “Y”> | | CH | |
| | | Heart | <S “Y”> | | HE | |
| | | Can't Move | <S “Y”> | | CM1 | |
| | | Can't walk | <S “Y”> | | CM2 | |
| | | Feel Strange | <S “Y”> | | FS1 | |
| | | Feel Funny | <S “Y”> | | FS2 | |
| | | Something | <S “Y”> | | FS3 | |
| | | Wrong | | | | |
| | | Don't Feel | <S “Y”> | | FS4 | |
| | | Right | | | | |
| | | Eye Problem | <S “Y”> | | EP | |
| | | <Other> | 17 | | | |
| 16 | <NO OTS> | | 20 | | | |
| 17 | <NRR> | | 500 | | | |
| | I think I'll first get | | | | | |
| | you to answer the | | | | | |
| | Quick Health | | | | | |
| | Checklist. | | | | | |
| 20 | <COMMENT: This | | 21 | | | |
| | section checks out | | | | | |
| | SHEs associated | | | | | |
| | with the reply given | | | | | |
| | by the client.> \|\| | | | | | |
| | <NO OTS> | | | | | |
| 21 | <NO OTS> | I#PA=Y | 40 | | | |
| | | I#IL=Y | 470 | | | |
| | | I#WE=Y | 350 | | | |
| | | I#NU=Y | 400 | | | |
| | | I#DI=Y | 200 | | | |
| | | I#BR1=Y | 690 | | | |
| | | I#FA=Y | <GOTO | | | |
| | | | IS#FA-1> | | | |
| | | I#TW1=Y | 460 | | | |
| | | I#CH=Y | 495 | | | |
| | | I#HE=Y | 495 | | | |
| | | I#CM1=Y | 650 | | | |
| | | I#CM2=Y | 670 | | | |
| | | I#FS1=Y | 428 | | | |
| | | I#FS2=Y | 428 | | | |
| | | I#FS3=Y | 428 | | | |
| | | I#FS4=Y | 428 | | | |
| | | I#LBA=Y | 460 | | | |
| | | I#LCO=Y | 460 | | | |
| | | I#EP=Y | 463 | | | |
| | | I#NAU=Y | 610 | | | |
| | | I#DIZ=Y | 610 | | | |
| | | I#LH=Y | 610 | | | |
| | | I#CS=Y | 610 | | | |
| | | I#DF1=Y | 620 | | | |
| | <COMMENT: If no | | 500 | | | |
| | SHE associated with | | | | | |
| | the one specific | | | | | |
| | problem, check see | | | | | |
| | if there could be | | | | | |
| | other problems. Go | | | | | |
| | to the Quick Health | | | | | |
| | Checklist.> \|\| | | | | | |
| | <NO OTS> | | | | | |
| 40 | <NRR> | | 45 | | | |
| | I want to find out | | | | | |
| | where the pain is. | | | | | |
| | I'm going to list one | | | | | |
| | location at a time. | | | | | |
| | After I say the | | | | | |
| | location, say either | | | | | |
| | Yes or No. | | | | | |
| 45 | <NO OTS> | | T3=1 | | | |
| 50 | Pain in the chest? | Yes | <S> \|\| 55 | | PCH | |
| | | No | <S> \|\| 65 | | PCH | |
| 55 | <NO OTS> | C2=1 | 60 | | | |
| | | C2><1 | C2=1\|\|<C | | | |
| | | | IS#HA-1> | | | |
| 60 | <NO OTS> | C3=1 | 65 | | | |
| | | C3><1 | C3=1\|\|<C | | | |
| | | | IS#CAE-1> | | | |
| 70 | Back? | Yes | <S> \|\| 80 | | PBA | |
| | | No | <S> \|\| 85 | | PBA | |
| 80 | <NO OTS> | C2=1 | 85 | | | |
| | | C2><1 | C2=1\|\|<C | | | |
| | | | IS#HA-1> | | | |
| 85 | Neck? | Yes | <S> \|\| 90 | | PNE | |
| | | No | <S> \|\| 95 | | PNE | |
| 90 | <NO OTS> | C2=1 | 95 | | | |
| | | C2><1 | C2=1\|\|<C | | | |
| | | | IS#HA-1> | | | |
| 95 | Jaw? | Yes | <S> \|\| 100 | | PJ | |
| | | No | <S> \|\| 105 | | PJ | |
| 100 | <NO OTS> | C2=1 | 105 | | | |
| | | C2><1 | C2=1\|\|<C | | | |
| | | | IS#HA-1> | | | |
| 105 | Stomach? | Yes | <S> \|\| 110 | | PST | |
| | | No | <S> \|\| 115 | | PST | |
| 110 | <NO OTS> | C2=1 | 115 | | | |
| | | C2><1 | C2=1\|\|<C | | | |
| | | | IS#HA-1> | | | |
| 115 | Both Shoulders? | Yes | <S> \|\| 120 | | PSH2 | |
| | | No | <S> \|\| 125 | | PSH2 | |
| 120 | <NO OTS> | C2=1 | 125 | | | |
| | | C2><1 | C2=1\|\|<C | | | |
| | | | IS#HA-1> | | | |
| 125 | One Shoulder? | Yes | <S> \|\| 130 | | PSH1 | |
| | | No | <S> \|\| 135 | | PSH1 | |
| 130 | <NO OTS> | C2=1 | 135 | | | |
| | | C2><1 | C2=1\|\|<C | | | |
| | | | IS#HA-1> | | | |
| 135 | Two Arms? | Yes | <S> \|\| 140 | | PA2 | |
| | | No | <S> \|\| 145 | | PA2 | |
| 140 | <NO OTS> | C2=1 | 145 | | | |
| | | C2><1 | C2=1\|\|<C | | | |
| | | | IS#HA-1> | | | |
| 145 | One Arm? | Yes | <S> \|\| 150 | | PA1 | |
| | | No | <S> \|\| 175 | | PA1 | |
| 150 | <NO OTS> | C1=1 | 155 | | | |
| | | C1><1 | C1=1\|\|<C | | | |
| | | | IS#S-1> | | | |
| 155 | <NO OTS> | C2=1 | 175 | | | |
| | | C2><1 | C2=1\|\|<C | | | |
| | | | IS#HA-1> | | | |
| 175 | Pain in head? | Yes | <S> \|\| 180 | | PH | |
| | | No | <S> \|\| 185 | | PH | |
| 180 | <NO OTS> | C1=1 | 185 | | | |
| | | C1><1 | C1=1\|\|<C | | | |
| | | | IS#S-1> | | | |
| 185 | Pain in Face? | Yes | <S> \|\| 190 | | PFA | |
| | | No | <S> \|\| 194 | | PFA | |
| 190 | <NO OTS> | C1=1 | 194 | | | |
| | | C1><1 | C1=1\|\|<C | | | |
| | | | IS#S-1> | | | |
| 194 | Pain in One Leg? | Yes | <S> \|\| 195 | | PL1 | |
| | | No | <S> \|\| 196 | | PL1 | |
| 195 | <NO OTS> | C1=1 | 196 | | | |
| | | C1><1 | C1=1\|\|<C | | | |
| | | | IS#S-1> | | | |
| 196 | Is the pain very bad? | No | <S> \|\| 199 | | PAB | |
| | | Yes | <S> \|\| 197 | | PAB | |
| 197 | Is the pain so bad | No | <S> \|\| 198 | | PACW | |
| | that you can't walk? | Yes | <S> \|\| <END> | | PACW | |
| 198 | Is the pain so bad | No | <S> \|\| 199 | | EM1 | |
| | that you want me to | Yes | <S> \|\| <END> | | EM1 | |
| | make an Emergency | | | | | |
| | Call? | | | | | |
| 199 | <NO OTS> | REG1=2 | <RETURN> | | | |
| | | T9=1 | <RETURN> | | | |
| | | T9><1 | 500 | | | |
| 200 | <NRR> | | 202 | | | |
| | I want to find out | | | | | |
| | where the discomfort | | | | | |
| | is. | | | | | |
| | I'm going to list one | | | | | |
| | location at a time. | | | | | |
| | After I say the | | | | | |
| | location, say either | | | | | |
| | Yes or No. | | | | | |
| 202 | <NO OTS> | | T7=1 | | | |
| 205 | Discomfort in the | Yes | <S> \|\| 210 | | DCH | |
| | chest? | No | <S> \|\| 215 | | DCH | |
| 210 | <NO OTS> | C2=1 | 212 | | | |
| | | C2><1 | C2=1\|\|<C | | | |
| | | | IS#HA-1> | | | |
| 212 | <NO OTS> | C3=1 | 215 | | | |
| | | C3><1 | C3=1\|\|<C | | | |
| | | | IS#CAE-1> | | | |
| 215 | Back? | Yes | <S> \|\| 217 | | DBA | |
| | | No | <S> \|\| 220 | | DBA | |
| 217 | <NO OTS> | C2=1 | 220 | | | |
| | | C2><1 | C2=1\|\|<C | | | |
| | | | IS#HA-1> | | | |
| 220 | Neck? | Yes | <S> \|\| 222 | | DNE | |
| | | No | <S> \|\| 225 | | DNE | |
| 222 | <NO OTS> | C2=1 | 225 | | | |
| | | C2><1 | C2=1\|\|<C | | | |
| | | | IS#HA-1> | | | |
| 225 | Jaw? | Yes | <S> \|\| 227 | | DJ | |
| | | No | <S> \|\| 30 | | DJ | |
| 227 | <NO OTS> | C2=1 | 230 | | | |
| | | C2><1 | C2=1\|\|<C | | | |
| | | | IS#HA-1> | | | |
| 230 | Stomach? | Yes | <S> \|\| 232 | | DST | |
| | | No | <S> \|\| 235 | | DST | |
| 232 | <NO OTS> | C2=1 | 235 | | | |
| | | C2><1 | C2=1\|\|<C | | | |
| | | | IS#HA-1> | | | |
| 235 | Both Shoulders? | Yes | <S> \|\| 237 | | DSH2 | |
| | | No | <S> \|\| 240 | | DSH2 | |
| 237 | <NO OTS> | C2=1 | 240 | | | |
| | | C2><1 | C2=1\|\|<C | | | |
| | | | IS#HA-1> | | | |
| 240 | One Shoulder? | Yes | <S> \|\| 242 | | DSH1 | |
| | | No | <S> \|\| 245 | | DSH1 | |
| 242 | <NO OTS> | C2=1 | 245 | | | |
| | | C2><1 | C2=1\|\|<C | | | |
| | | | IS#HA-1> | | | |
| 245 | Two Arms? | Yes | <S> \|\| 247 | | DA2 | |
| | | No | <S> \|\| 250 | | DA2 | |
| 247 | <NO OTS> | C2=1 | 250 | | | |
| | | C2><1 | C2=1\|\|<C | | | |
| | | | IS#HA-1> | | | |
| 250 | One Arm? | Yes | <S> \|\| 252 | | DA1 | |
| | | No | <S> \|\| 257 | | DA1 | |
| 252 | <NO OTS> | C1=1 | 255 | | | |
| | | C1><1 | C1=1\|\|<C | | | |
| | | | IS#S-1> | | | |
| 255 | <NO OTS> | C2=1 | 257 | | | |
| | | C2><1 | C2=1\|\|<C | | | |
| | | | IS#HA-1> | | | |
| 257 | Discomfort in head? | Yes | <S> \|\| 260 | | DH | |
| | | No | <S> \|\| 262 | | DH | |
| 260 | <NO OTS> | C1=1 | 262 | | | |
| | | C1><1 | C1=1\|\|<C | | | |
| | | | IS#S-1> | | | |
| 262 | Discomfort in Face? | Yes | <S> \|\| 265 | | DFA | |
| | | No | <S> \|\| 267 | | DFA | |
| 265 | <NO OTS> | C1=1 | 267 | | | |
| | | C1><1 | C1=1\|\|<C | | | |
| | | | IS#S-1> | | | |
| 267 | Discomfort in One | Yes | <S> \|\| 270 | | DL1 | |
| | Leg? | No | <S> \|\| 272 | | DL1 | |
| 270 | <NO OTS> | C1=1 | 272 | | | |
| | | C1><1 | C1=1\|\|<C | | | |
| | | | IS#S-1> | | | |
| 272 | Is the discomfort | No | <S> \|\| 280 | | DIB | |
| | very bad? | Yes | <S> \|\| 275 | | DIB | |
| 275 | Is the discomfort so | No | <S> \|\| 277 | | DICW | |
| | bad that you can't | Yes | <S> \|\| <END> | | DICW | |
| | walk? | | | | | |
| 277 | Is the discomfort so | No | <S> \|\| 280 | | EM1 | |
| | bad that you want | Yes | <S> \|\| <END> | | EM1 | |
| | me to make an | | | | | |
| | Emergency Call? | | | | | |
| 280 | <NO OTS> | REG1=2 | <RETURN> | | | |
| | | T9=1 | <RETURN> | | | |
| | | T9><1 | 500 | | | |
| 350 | <NRR> | | 354 | | | |
| | I want to find out | | | | | |
| | where the weakness | | | | | |
| | is. | | | | | |
| | I'm going to ask you | | | | | |
| | a few questions. | | | | | |
| 354 | <NO OTS> | | T5=1 | | | |
| 355 | Do you have | Yes | 360 | | | |
| | weakness in the arm | No | <S> \|\| 365 | | WAR | |
| | or arms? | | | | | |
| 360 | Left, right or both? | Left | <S> | | WAR | |
| | | Right | <S> | | WAR | |
| | | Both | <S> | | WAR | |
| 365 | Weakness in the leg | Yes | 370 | | | |
| | or legs? | No | <S> \|\| 375 | | WLE | |
| 370 | Left, right or both? | Left | <S> | | WLE | |
| | | Right | <S> | | WLE | |
| | | Both | <S> | | WLE | |
| 375 | Weakness in face or | Yes | 380 | | | |
| | mouth? | No | <S> \|\| 385 | | WFA | |
| 380 | Both sides, left side | Both | <S> | | WFA | |
| | only, or right side | Left | <S> | | WFA | |
| | only? | Right | <S> | | WFA | |
| 385 | <NO OTS> | C1=1 | 390 | | | |
| 387 | <NO OTS> | I#WAR=L | C1=1\|\|<C | | | |
| | | | IS#S-1> | | | |
| | | I#WAR=R | C1=1\|\|<C | | | |
| | | | IS#S-1> | | | |
| | | I#WLE=L | C1=1\|\|<C | | | |
| | | | IS#S-1> | | | |
| | | I#WLE=R | C1=1\|\|<C | | | |
| | | | IS#S-1> | | | |
| | | I#WFA=L | C1=1\|\|<C | | | |
| | | | IS#S-1> | | | |
| | | I#WFA=R | C1=1\|\|<C | | | |
| | | | IS#S-1> | | | |
| 390 | Is the weakness very | No | <S> \|\| 395 | | WEB | |
| | bad? | Yes | <S> \|\| 391 | | WEB | |
| 391 | Is the weakness so | No | <S> \|\| 392 | | WECW | |
| | bad that you can't | Yes | <S> \|\| <END> | | WECW | |
| | walk? | | | | | |
| 392 | Is the weakness so | No | <S> \|\| 395 | | EM1 | |
| | bad that you want | Yes | <S> \|\| <END> | | EM1 | |
| | me to make an | | | | | |
| | Emergency Call? | | | | | |
| 395 | <NO OTS> | REG1=2 | <RETURN> | | | |
| | | T9=1 | <RETURN> | | | |
| | | T9><1 | 500 | | | |
| 400 | <NRR> | | 404 | | | |
| | I want to find out | | | | | |
| | where the numbness | | | | | |
| | is. | | | | | |
| | I'm going to ask you | | | | | |
| | a few questions. | | | | | |
| 404 | <NO OTS> | | T6=1 | | | |
| 405 | Do you have | Yes | 410 | | | |
| | numbness in the arm | No | <S> \|\| 415 | | NAR | |
| | or arms? | | | | | |
| 410 | Left, right or both? | Left | <S> | | NAR | |
| | | Right | <S> | | NAR | |
| | | Both | <S> | | NAR | |
| 415 | Numbness in the leg | Yes | 420 | | | |
| | or legs? | No | <S> \|\| 423 | | NLE | |
| 420 | Left, right or both? | Left | <S> | | NLE | |
| | | Right | <S> | | NLE | |
| | | Both | <S> | | NLE | |
| 423 | Numbness in face or | Yes | 424 | | | |
| | mouth? | No | <S> \|\| 425 | | NFA | |
| 424 | Both sides, left side | Both | <S> | | NFA | |
| | only, or right side | Left | <S> | | NFA | |
| | only? | Right | <S> | | NFA | |
| 425 | <NO OTS> | C1=1 | 427 | | | |
| 426 | <NO OTS> | I#NAR=L | C1=1\|\|<C | | | |
| | | | IS#S-1> | | | |
| | | I#NAR=R | C1=1\|\|<C | | | |
| | | | IS#S-1> | | | |
| | | I#NLE=L | C1=1\|\|<C | | | |
| | | | IS#S-1> | | | |
| | | I#NLE=R | C1=1\|\|<C | | | |
| | | | IS#S-1> | | | |
| | | I#NFA=L | C1=1\|\|<C | | | |
| | | | IS#S-1> | | | |
| | | I#NFA=R | C1=1\|\|<C | | | |
| | | | IS#S-1> | | | |
| 427 | <NO OTS> | REG1=2 | <RETURN> | | | |
| | | T9=1 | <RETURN> | | | |
| | | T9><1 | 500 | | | |
| 428 | <NRR> | | 429 | | | |
| | I want to find out | | | | | |
| | where the strange | | | | | |
| | feeling is. | | | | | |
| | I'm going to list one | | | | | |
| | location at a time. | | | | | |
| | After I say the | | | | | |
| | location, say either | | | | | |
| | Yes or No. | | | | | |
| 429 | <NO OTS> | | T12=1 | | | |
| 430 | In the chest? | Yes | <S> \|\| 431 | | FCH | |
| | | No | <S> \|\| 433 | | FCH | |
| 431 | <NO OTS> | C2=1 | 432 | | | |
| | | C2><1 | C2=1\|\|<C | | | |
| | | | IS#HA-1> | | | |
| 432 | <NO OTS> | C3=1 | 433 | | | |
| | | C3><1 | C3=1\|\|<C | | | |
| | | | IS#CAE-1> | | | |
| 433 | Back? | Yes | <S> \|\| 434 | | FBA | |
| | | No | <S> \|\| 435 | | FBA | |
| 434 | <NO OTS> | C2=1 | 435 | | | |
| | | C2><1 | C2=1\|\|<C | | | |
| | | | IS#HA-1> | | | |
| 435 | Neck? | Yes | <S> \|\| 436 | | FNE | |
| | | No | <S> \|\| 437 | | FNE | |
| 436 | <NO OTS> | C2=1 | 437 | | | |
| | | C2><1 | C2=1\|\|<C | | | |
| | | | IS#HA-1> | | | |
| 437 | Jaw? | Yes | <S> \|\| 438 | | FJ | |
| | | No | <S> \|\| 439 | | FJ | |
| 438 | <NO OTS> | C2=1 | 439 | | | |
| | | C2><1 | C2=1\|\|<C | | | |
| | | | IS#HA-1> | | | |
| 439 | Stomach? | Yes | <S> \|\| 440 | | FST | |
| | | No | <S> \|\| 441 | | FST | |
| 440 | <NO OTS> | C2=1 | 441 | | | |
| | | C2><1 | C2=1\|\|<C | | | |
| | | | IS#HA-1> | | | |
| 441 | Both Shoulders? | Yes | <S> \|\| 442 | | FSH2 | |
| | | No | <S> \|\| 443 | | FSH2 | |
| 442 | <NO OTS> | C2=1 | 443 | | | |
| | | C2><1 | C2=1\|\|<C | | | |
| | | | IS#HA-1> | | | |
| 443 | One Shoulder? | Yes | <S> \|\| 444 | | FSH1 | |
| | | No | <S> \|\| 445 | | FSH1 | |
| 444 | <NO OTS> | C2=1 | 445 | | | |
| | | C2><1 | C2=1\|\|<C | | | |
| | | | IS#HA-1> | | | |
| 445 | Two Arms? | Yes | <S> \|\| 446 | | FA2 | |
| | | No | <S> \|\| 447 | | FA2 | |
| 446 | <NO OTS> | C2=1 | 447 | | | |
| | | C2><1 | C2=1\|\|<C | | | |
| | | | IS#HA-1> | | | |
| 447 | One Arm? | Yes | <S> \|\| 448 | | FA1 | |
| | | No | <S> \|\| 450 | | FA1 | |
| 448 | <NO OTS> | C1=1 | 449 | | | |
| | | C1><1 | C1=1\|\|<C | | | |
| | | | IS#S-1> | | | |
| 449 | <NO OTS> | C2=1 | 450 | | | |
| | | C2><1 | C2=1\|\|<C | | | |
| | | | IS#HA-1> | | | |
| 450 | In the head? | Yes | <S> \|\| 451 | | FH | |
| | | No | <S> \|\| 452 | | FH | |
| 451 | <NO OTS> | C1=1 | 452 | | | |
| | | C1><1 | C1=1\|\|<C | | | |
| | | | IS#S-1> | | | |
| 452 | In the Face? | Yes | <S> \|\| 453 | | FFA | |
| | | No | <S> \|\| 454 | | FFA | |
| 453 | <NO OTS> | C1=1 | 454 | | | |
| | | C1><1 | C1=1\|\|<C | | | |
| | | | IS#S-1> | | | |
| 454 | In One Leg? | Yes | <S> \|\| 455 | | FL1 | |
| | | No | <S> \|\| 456 | | PL1 | |
| 455 | <NO OTS> | C1=1 | 456 | | | |
| | | C1><1 | C1=1\|\|<C | | | |
| | | | IS#S-1> | | | |
| 456 | Is the strange feeling | No | <S> \|\| 459 | | FSB | |
| | very bad? | Yes | <S> \|\| 457 | | FSB | |
| 457 | Is the strange feeling | No | <S> \|\| 458 | | FSCW | |
| | so bad that you can't | Yes | <S> \|\| <END> | | FSCW | |
| | walk? | | | | | |
| 458 | Is the strange feeling | No | <S> \|\| 459 | | EM1 | |
| | so bad that you want | Yes | <S> \|\| <END> | | EM1 | |
| | me to make an | | | | | |
| | Emergency Call? | | | | | |
| 459 | <NO OTS> | REG1=2 | <RETURN> | | | |
| | | T9=1 | <RETURN> | | | |
| | | T9><1 | 500 | | | |
| 460 | <COMMENT: If | | C9=1 | | | |
| | client has trouble | | | | | |
| | walking, loss of | | | | | |
| | balance, or loss of | | | | | |
| | coordination, check | | | | | |
| | for Stroke.> \|\| | | | | | |
| | <NO OTS> | | | | | |
| 461 | <NO OTS> | C1=1 | 462 | | | |
| | | C1><1 | C1=1\|\|<C | | | |
| | | | IS#S-1> | | | |
| 462 | <NO OTS> | REG1=2 | <RETURN> | | | |
| | | T9=1 | <RETURN> | | | |
| | | T9><1 | 500 | | | |
| 463 | <COMMENT: If | | T14=1 | | | |
| | client has sudden eye | | | | | |
| | problems, check for | | | | | |
| | Stroke.> \|\| | | | | | |
| | <NO OTS> | | | | | |
| 464 | <NO OTS> | C1=1 | 465 | | | |
| | | C1><1 | C1=1\|\|<C | | | |
| | | | IS#S-1> | | | |
| 465 | <NO OTS> | REG1=2 | <RETURN> | | | |
| | | T9=1 | <RETURN> | | | |
| | | T9><1 | 500 | | | |
| 470 | <NRR> | | 472 | | | |
| | I want to find out | | | | | |
| | more about your | | | | | |
| | illness. | | | | | |
| | I'm going to list one | | | | | |
| | location at a time. | | | | | |
| | After I say the | | | | | |
| | location, say either | | | | | |
| | Yes or No. | | | | | |
| 472 | <NO OTS> | | T10=1 | | | |
| 474 | Ill in the stomach? | Yes | <S> \|\| 476 | | IST | |
| | | No | <S> \|\| 478 | | IST | |
| 476 | <NO OTS> | C2=1 | 478 | | | |
| | | C2><1 | C2=1\|\|<C | | | |
| | | | IS#HA-1> | | | |
| 478 | Ill in the chest? | Yes | <S> \|\| 480 | | ICH | |
| | | No | <S> \|\| 484 | | ICH | |
| 480 | <NO OTS> | C2=1 | 482 | | | |
| | | C2><1 | C2=1\|\|<C | | | |
| | | | IS#HA-1> | | | |
| 482 | <NO OTS> | C3=1 | 484 | | | |
| | | C3><1 | C3=1\|\| | | | |
| | | | <C | | | |
| | | | IS#CAE-1> | | | |
| 484 | Ill in the head? | Yes | <S> \|\| 486 | | IH | |
| | | No | <S> \|\| 488 | | IH | |
| 486 | <NO OTS> | C1=1 | 488 | | | |
| | | C1><1 | C1=1\|\|<C | | | |
| | | | IS#S-1> | | | |
| 488 | Is the illness very | No | <S> \|\| 494 | | ILB | |
| | bad? | Yes | <S> \|\| 490 | | ILB | |
| 490 | Is the illness so bad | No | <S> \|\| 492 | | ILCW | |
| | that you can't walk? | Yes | <S> \|\| <END> | | ILCW | |
| 492 | Is the illness so bad | No | <S> \|\| 494 | | EM1 | |
| | that you want me to | Yes | <S> \|\| <END> | | EM1 | |
| | make an Emergency | | | | | |
| | Call? | | | | | |
| 494 | <NO OTS> | REG1=2 | <RETURN> | | | |
| | | T9=1 | <RETURN> | | | |
| | | T9><1 | 500 | | | |
| 495 | <COMMENT: If the | | | | | |
| | client complains | | | | | |
| | about his/her chest | | | | | |
| | or heart, he/she is | | | | | |
| | checked for Heart | | | | | |
| | Attack and EWSs of | | | | | |
| | Cardiac Arrest.> \|\| | | | | | |
| | <NO OTS> | | | | | |
| 496 | <NO OTS> | C2=1 | 497 | | | |
| | | C2><1 | C2=1\|\|<C | | | |
| | | | IS#HA-1> | | | |
| 497 | <NO OTS> | C3=1 | 498 | | | |
| | | C3><1 | C3=1\|\|<C | | | |
| | | | IS#CAE-1> | | | |
| 498 | <NO OTS> | REG1=2 | <RETURN> | | | |
| | | T9=1 | <RETURN> | | | |
| | | T9><1 | 500 | | | |
| 500 | <NRR> | | 510 | | | |
| | OK, I now want to | | | | | |
| | ask you a few | | | | | |
| | general health | | | | | |
| | questions. | | | | | |
| | After I say a health | | | | | |
| | condition, please | | | | | |
| | reply with: “No or | | | | | |
| | Yes”. | | | | | |
| 510 | Question 1: Any | No | <S> \|\| 515 | | PA | |
| | sudden pain? | Yes | <S> \|\| 515 | | PA | |
| 515 | Any sudden illness? | No | <S> \|\| 520 | | IL | |
| | | Yes | <S> \|\| 520 | | IL | |
| 520 | Any sudden | No | <S> \|\| 525 | | WE | |
| | weakness? | Yes | <S> \|\| 525 | | WE | |
| 525 | Any sudden | No | <S> \|\| 530 | | NU | |
| | numbness? | Yes | <S> \|\| 530 | | NU | |
| 530 | Any sudden | No | <S> \|\| 535 | | DI | |
| | discomfort? | Yes | <S> \|\| 535 | | DI | |
| 535 | Sudden breathing | No | <S> \|\| 540 | | BR1 | |
| | problem? | Yes | <S> \|\| 540 | | BR1 | |
| 540 | Sudden trouble with | No | <S> \|\| 545 | | LBA | |
| | balance? | Yes | <S> \|\| 545 | | LBA | |
| 545 | Sudden trouble with | No | <S> \|\| 550 | | LCO | |
| | coordination? | Yes | <S> \|\| 550 | | LCO | |
| 550 | Sudden trouble with | No | <S> \|\| 555 | | EP | |
| | eyesight? | Yes | <S> \|\| 555 | | EP | |
| 555 | Anything that feels | No | <S> \|\| 556 | | FS1 | |
| | “strange”? | Yes | <S> \|\| 556 | | FS1 | |
| 556 | Do you suddenly | No | <S> \|\| 557 | | NAU | |
| | have nausea? | Yes | <S> \|\| 557 | | NAU | |
| 557 | Sudden dizziness? | No | <S> \|\| 558 | | DIZ | |
| | | Yes | <S> \|\| 558 | | DIZ | |
| 558 | Suddenly | No | <S> \|\| 559 | | LH | |
| | lightheaded? | Yes | <S> \|\| 559 | | LH | |
| 559 | Sudden cold sweat? | No | <S> \|\| 560 | | CS | |
| | | Yes | <S> \|\| 560 | | CS | |
| 560 | Sudden droopy | No | <S> \|\| 561 | | DF1 | |
| | face? | Yes | <S> \|\| 561 | | DF1 | |
| 561 | Can you walk OK? | Yes | <S “N”> \|\| | | TW1 | |
| | | | 565 | | | |
| | | No | <S “Y”> \|\| | | TW1 | |
| | | | 565 | | | |
| 565 | <NO OTS> | | T9=1 \|\| 570 | | | |
| 570 | <COMMENT: This | | | | | |
| | section gets more | | | | | |
| | health related | | | | | |
| | information, based | | | | | |
| | on the replies | | | | | |
| | associated with the | | | | | |
| | Quick Checklist.> | | | | | |
| 572 | <NO OTS> | T3=1 | 574 | | | |
| | | I#PA=Y | <C IU#40> | | | |
| 573 | <NO OTS> | T10=1 | 574 | | | |
| | | I#IL=Y | <C IU#470> | | | |
| 574 | <NO OTS> | T5=1 | 576 | | | |
| | | I#WE=Y | <C IU#350> | | | |
| 576 | <NO OTS> | T6=1 | 578 | | | |
| | | I#NU=Y | <C IU#400> | | | |
| 578 | <NO OTS> | T7=1 | 580 | | | |
| | | I#DI=Y | <C IU#200> | | | |
| 580 | <NO OTS> | C9=1 | 582 | | | |
| | | I#TW1=Y | <C IU#460> | | | |
| 582 | <NO OTS> | T10=1 | 584 | | | |
| | | I#IL=Y | <C IU#470> | | | |
| 584 | <NO OTS> | T11=1 | 585 | | | |
| | | I#BR1=Y | <C IU#690> | | | |
| 585 | <NO OTS> | C9=1 | 586 | | | |
| | | I#LBA=Y | <C IU#460> | | | |
| | | I#LCO=Y | <C IU#460> | | | |
| 586 | <NO OTS> | T14=1 | 587 | | | |
| | | I#EP=Y | <C IU#463> | | | |
| 587 | <NO OTS> | T12=1 | 588 | | | |
| | | I#FS1=Y | <C IU#428> | | | |
| 588 | <NO OTS> | T17=1 | 589 | | | |
| | | I#NAU=Y | <C IU#610> | | | |
| 589 | <NO OTS> | T17=1 | 590 | | | |
| | | I#DIZ=Y | <C IU#610> | | | |
| 590 | <NO OTS> | T17=1 | 591 | | | |
| | | I#LH=Y | <C IU#610> | | | |
| 591 | <NO OTS> | T17=1 | 592 | | | |
| | | I#CS=Y | <C IU#610> | | | |
| 592 | <NO OTS> | T18 | 600 | | | |
| | | I#DF1=Y | <C IU#620> | | | |
| 600 | <COMMENT: No | | 900 | | | |
| | specific SHEs have | | | | | |
| | been detected. Go to | | | | | |
| | the General SHE | | | | | |
| | Check-up section.> | | | | | |
| | \|\| | | | | | |
| | <NO OTS> | | | | | |
| 610 | <COMMENT: | C2=1 | 614 | | | |
| | Check for Heart | C2><1 | C2=1 \|\| <C | | | |
| | Attack.> \|\| | | IS#HA-1> | | | |
| | <NO OTS> | | | | | |
| 614 | <NO OTS> | | T17=1 | | | |
| 616 | <NO OTS> | REG1=2 | <RETURN> | | | |
| | | T9=1 | <RETURN> | | | |
| | | T9><1 | 500 | | | |
| 620 | <COMMENT: | C1=1 | 624 | | | |
| | Check for Heart | C1><1 | C1=1 \|\| <C | | | |
| | Attack.> \|\| | | IS#S-1> | | | |
| | <NO OTS> | | | | | |
| 624 | <NO OTS> | | T18=1 | | | |
| 626 | <NO OTS> | REG1=2 | <RETURN> | | | |
| | | T9=1 | <RETURN> | | | |
| | | T9><1 | 500 | | | |
| 650 | <COMMENT: This | | 660 | | | |
| | part checks | | | | | |
| | situations when the | | | | | |
| | client says that | | | | | |
| | he/she cannot | | | | | |
| | move.> \|\| | | | | | |
| | <NRR> | | | | | |
| 660 | What is the reason | Pain | <S “Y”> | | PA | |
| | that you can't move? | Illness | <S “Y”> | | IL | |
| | | Weak | <S “Y”> | | WE | |
| | | Can't Walk | <S “Y”> | | CW | |
| | | Dizzy | <S “Y”> | | DIZ | |
| | | <Other> | 665 | | | |
| 665 | <NRR> | | | | | |
| | I will make an | | <S “Y”> \|\| | | EMC | |
| | Emergency call right | | <END> | | M | |
| | now. | | | | | |
| 670 | <COMMENT: This | | 675 | | | |
| | part checks | | | | | |
| | situations when the | | | | | |
| | client cannot walk.> | | | | | |
| | \|\| | | | | | |
| | <NRR> | | | | | |
| 675 | What is the reason | Pain | <S “Y”> | | PA | |
| | that you can't walk? | Illness | <S “Y”> | | IL | |
| | | Weak | <S “Y”> | | WE | |
| | | Can't Walk | <S “Y”> | | CW | |
| | | Dizzy | <S “Y”> | | DIZ | |
| | | <Other> | 680 | | | |
| 680 | <NRR> | | | | | |
| | I will make an | | <S “Y”> \|\| | | EMC | |
| | Emergency call right | | <END> | | W | |
| | now. | | | | | |
| 690 | <COMMENT: This | | 691 | | | |
| | section looks into | | | | | |
| | breathing related | | | | | |
| | problems.> \|\| | | | | | |
| | <NO OTS> | | | | | |
| 691 | <NO OTS> | | T11=1 | | | |
| 692 | Are you short of | Yes | <S> \|\| 694 | | BRS | |
| | breath? | No | <S> \|\| 698 | | BRS | |
| 694 | <NO OTS> | C2=1 | 698 | | | |
| | | C2><1 | C2=1\|\|<C | | | |
| | | | IS#HA-1> | | | |
| 698 | <NO OTS> | REG1=2 | <RETURN> | | | |
| | | T9=1 | <RETURN> | | | |
| | | T9><1 | 500 | | | |
| 700 | <COMMENT: | | | | | |
| | Handling | | | | | |
| | “Emergency Now”> | | | | | |
| | \|\| | | | | | |
| | <NO OTS> | | | | | |
| | <NRR> | | <S “Y”> \|\| | | EMN | |
| | I am making an | | <END> | | | |
| | Emergency call right | | | | | |
| | now. | | | | | |
| 900 | <COMMENT This | | 905 | | | |
| | section is carried out | | | | | |
| | if no specific SHE | | | | | |
| | was detected —It | | | | | |
| | checks for a General | | | | | |
| | SHE.> \|\| <NRR> | | | | | |
| 905 | Do you feel that you | Yes | 910 | | EMG | |
| | are in an Emergency | | <SAVE> \|\| | | | |
| | situation? | No | 920 | | EMG | |
| | | | <SAVE> \|\| | | | |
| | | Not Sure | 915 | | | |
| 910 | <NRR> | | <S “Y”> \|\| | | EMG | |
| | I am calling | | <END> | | [General | |
| | Emergency | | | | Emergency, | |
| | Response people | | | | per | |
| | right now. They will | | | | Client] | |
| | be calling you | | | | | |
| | shortly. | | | | | |
| 915 | <NRR> | | <R1SAVE | | | |
| | That's all for now. I | | “0”> \|\| | | | |
| | will check in on you | | <WAIT-600s | | | |
| | shortly. | | IS#M-2> \|\| | | | |
| | | | <END> | | | |
| 920 | <NRR> | | <R1SAVE | | | |
| | That's all for now. | | “0”> \|\| | | | |
| | You don't seem to | | <END> | | | |
| | have any serious | | | | | |
| | problem. If anything | | | | | |
| | comes up, just let | | | | | |
| | me know. Or press | | | | | |
| | the Emergency | | | | | |
| | Button if it is very | | | | | |
| | serious. | | | | | |

The master probe IS, M-1, starts when a trigger is detected. The M-1 carries out the following when a trigger condition occurs.

1) Information Gathering (Probe). This involves gathering additional information from the client, that is associated with the trigger condition.

2) Analysis. Determine if the trigger condition and additional information could be associated with one or more potential SHEs. If more than one, determine the priority of the SHEs. If there is at least one possible SHE, go to 3). If there are none, go to 4).

3) SHE Check. If there is an identified possible SHE, check if the client is experiencing it. This involves verbally interacting with the client. If an SHE is detected, the ED Mode takes over. If everything appears fine, check for the other identified potential SHEs if there are any more. If everything appears fine, go to 4).

4) Quick Health Checklist. The client is asked several standard questions from a health checklist.

5) Repeat Analysis & SHE Check. If any health related issues come out of the Checklist routine, then repeat steps 1), 2) and 3). That is:
Gather more information
Analyze the information to determine if there could be any possible SHEs
Check for these SHEs

6) General SHE Check. If nothing detected, then check with the client to see if the client feels that the present situation is an Emergency. If the client feels this way, then a General SHE is detected, and the emergency services are contacted.

7) Follow-up Check. If everything is OK, then do a quick follow-up a short time later. This is done by activating IS#M-2 (described further below) to start up, such as 15 minutes later.

In addition to the above, M-1 also carries out checks on a few SHEs:
Can't Move/Can't Walk
Breathing Problem
Severe Pain/Illness/Weakness

In some embodiments, the system operates as follows.

a) The system is always listening to the client. If the client says something that indicates a potential problem, or could indicate a potential problem, the apparatus starts up M-1.

b) In addition, the system periodically carries out a quick routine check. conversation. If the check identifies a potential problem, the apparatus starts up M-1.

c) M-1 asks the client a few questions to help determine if the client may be in a potential emergency situation.

d) If M-1 determines, or is informed, that the client has an early warning sign of one of the specific SHEs, e.g., heart attack, stroke, loss of consciousness, it does the following:
determine all the potential SHEs associated with the early warning sign
If only one, get the system to ask further questions regarding the SHE
If greater than one, determine which SHE is most probable, and get system to carry out the conversation associated with the most probable SHE
Then carry out any other SHE conversations after the most probably SHE has been examined
If a specific SHE is detected, auto-alert emergency response personnel
If no specific SHE is detected, M-1 checks for general SHEs
If nothing detected, but there is some uncertainty, instruct the apparatus to start up a check up query, M-2, in the near future
If everything is OK, end M-1

e) If, when carrying out a specific query, such as a stroke query (S-1), or heart attack query (HA-1), it is determined, or felt, that a follow-up check is required, arrange to have an appropriate check up query, such as a check up stroke query (S-2), check up heart attack (HA-1-2 or HA-2) started up in the future.
At the time check up conversation is to start, initiate the follow-up or check up conversation.
If an emergency situation detected, auto-alert emergency response personnel.

f) If at any time, during any conversation, the client has trouble responding properly to a question, begin a loss of understanding/responsiveness query (LOS-1) and analyze the situation.
If the client does not respond to inquiries, over a period of time, LOS-1 performs analysis to determine if the client is in an emergency situation
If the client starts to give incorrect or inappropriate responses to inquiries, LOS-1 performs analysis to determine if the person is in an emergency situation

g) If at any time, the client asks for help, or says “Emergency”, the system immediately calls for help. The apparatus can first quickly ask the client to confirm that it is an emergency situation. This is to prevent false alarms.

h) If, during a conversation, the client asks for Help, or says “Emergency”, the apparatus immediately interrupts the conversation, and calls for help. The system can first quickly ask the client to confirm that it is an emergency situation. This is to prevent false alarms.

These conversations and their details are described below.

As noted, the M-1 is started up by various Probe Trigger Conditions:

a) Client says “Help” or “Emergency”

b) Client says a health related word, on his/her own (e.g., pain)

c) Client says “Emergency Now”

d) Client indicated a problem (or several) during the Routine Check-up PVIS

e) Client directly indicated a problem during the Routine Check-up PVIS

f) A health-related sound

g) A health-related image

h) A significant physiological parameter value

The triggers that trigger a probe are listed in a probe trigger table, such as Table 22.

TABLE 22

| | | | | | “Currently |
|---|---|---|---|---|---|
| | | | | Interaction | Being |
| | PT | | | Session | Addressed” |
| PTC | Priority | PT Condition Description | PT Condition | (IS) # | Flag |
| C20 | P9 | {Client says, “Help”} | CIIC# C20 | M-1 | |
| C21 | P9 | Emergency | CIIC# C21 | M-1 | |
| C22 | P9 | Emergency AND Now | CIIC# C22 | M-1 | |
| C23 | P7 | Pain | CIIC# C23 | M-1 | |
| C24 | P7 | Ill | CIIC# C24 | M-1 | |
| C25 | P7 | Not AND Well | CIIC# C25 | M-1 | |
| C26 | P7 | Weak | CIIC# C26 | M-1 | |
| C27 | P7 | Numb | CIIC# C27 | M-1 | |
| C28 | P7 | Discomfort | CIIC# C28 | M-1 | |
| C29 | P7 | Pressure | CIIC# C29 | M-1 | |
| C30 | P7 | Fullness | CIIC# C30 | M-1 | |
| C40 | P7 | Squeezing | CIIC# C40 | M-1 | |
| C41 | P7 | Feel AND Strange | CIIC# C41 | M-1 | |
| C42 | P7 | Feel AND Funny | CIIC# C42 | M-1 | |
| C43 | P7 | Something AND Wrong | CIIC# C43 | M-1 | |
| C44 | P7 | Doesn't AND Feel AND | CIIC# C44 | M-1 | |
| | | Right | | | |
| C45 | P7 | Breathe | CIIC# C45 | M-1 | |
| C46 | P7 | Breath | CIIC# C46 | M-1 | |
| C47 | P7 | Breathing | CIIC# C47 | M-1 | |
| C48 | P7 | Trouble AND Walking | CIIC# C48 | M-1 | |
| C49 | P7 | Poor AND Balance | CIIC# C49 | M-1 | |
| C50 | P7 | Poor AND Coordination | CIIC# C50 | M-1 | |
| C60 | P7 | Eye AND Problem | CIIC# C60 | M-1 | |
| C61 | P7 | Trouble AND Seeing | CIIC# C61 | M-1 | |
| C62 | P7 | Trouble AND Speaking | CIIC# C62 | M-1 | |
| C63 | P7 | Can't AND Move | CIIC# C63 | M-1 | |
| C64 | P7 | Can't AND Walk | CIIC# C64 | M-1 | |
| C65 | P7 | Chest AND Problem | CIIC# C65 | M-1 | |
| C66 | P7 | Heart AND Problem | CIIC# C66 | M-1 | |
| C67 | P7 | Dizzy | CIIC# C67 | M-1 | |
| C68 | P7 | Dizziness | CIIC# C68 | M-1 | |
| C69 | P7 | Face AND Droopy | CIIC# C69 | M-1 | |
| C70 | P7 | Mouth AND Droopy | CIIC# C70 | M-1 | |
| C71 | P7 | Headache | CIIC# C71 | M-1 | |
| C72 | P7 | Nauseous | CIIC# C72 | M-1 | |
| C73 | P7 | Lightheaded | CIIC# C73 | M-1 | |
| C74 | P7 | Cold AND Sweat | CIIC# C74 | M-1 | |
| C75 | P7 | Hurts | CIIC# C75 | M-1 | |
| C76 | P7 | I AND Fell | CIIC# C76 | M-1 | |
| C77 | P3 | Attention | CIIC# C77 | M-1 | |
| C78 | P3 | Ed | CIIC# C78 | M-1 | |
| C79 | P3 | Edie | CIIC# C79 | M-1 | |
| P100 | P7 | Heart Rate - Low (below | HL1E | MPP-1 | |
| | | Level 1) - ECG Monitor | | | |
| P101 | P7 | Heart Rate - Low (below | HL1M | MPP-1 | |
| | | Level 1) - Heart Rate | | | |
| | | Monitor | | | |
| P102 | P7 | Heart Rate - Low (below | HL1B | MPP-1 | |
| | | Level 1) - Pulse | | | |
| | | Oximeter | | | |
| P103 | P7 | Respiratory Rate - Low | RL1E | MPP-1 | |
| | | (Below Level 1) - ECG | | | |
| | | Monitor | | | |
| P104 | P7 | Respiratory Rate - Low | RL1B | MPP-1 | |
| | | (Below Level 1) - Pulse | | | |
| | | Oximeter | | | |
| P105 | P7 | Blood Oxygen Saturation - | BOL1 | MPP-1 | |
| | | Low (Below Level 1) | | | |
| P106 | P7 | Blood Pressure- Low | BPL1 | MPP-1 | |
| | | (Below Level 1) | | | |
| P107 | P7 | Fall Detection Monitor | FDM | MPP-1 | |
| | | has detected a fall. | | | |
| P108 | P7 | ECG Signal slightly bad | ECB1 | MPP-1 | |
| P109 | P7 | ECG Signal very bad | ECB9 | MPP-1 | |
| PAS1 | P7 | Client makes cries of | S#PAS1=Y | MS-1 | |
| | | pain | | | |
| PAS2 | P7 | Client says “ouch” | S#PAS2=Y | MS-1 | |
| FAS | P7 | Sound of falling detected | S#FAS1=Y | MS-1 | |
| EMK | P7 | Client indicates | S#EMK=Y | MS-1 | |
| | | Emergency through non- | | | |
| | | verbal means - Knocking | | | |
| EMY | P7 | Client indicates | S#EMY=Y | MS-1 | |
| | | Emergency through non- | | | |
| | | verbal means - Yelping | | | |
| FAV | P7 | Video Monitor detects | V#FAV=Y | MV-1 | |
| | | client falling. | | | |
| DF1 | P7 | Video Monitor detects | V#DF1=Y | MV-1 | |
| | | droopy face. | | | |
| DF2 | P7 | Video Monitor detects | V#DF2=Y | MV-1 | |
| | | droopy mouth. | | | |
| TWV | P7 | Video Monitor detects | V#TWV=Y | MV-1 | |
| | | trouble walking. | | | |
| EMW | P7 | Client indicates | V#EMW=Y | MV-1 | |
| | | Emergency through non- | | | |
| | | verbal means - Waving | | | |
| | | arm | | | |
| EML | P7 | Client indicates | VS#EML=Y | MV-1 | |
| | | Emergency through non- | | | |
| | | verbal means - Lifting | | | |
| | | leg | | | |
| W1 | P5 | Start up the IS at time: | {Time = | S-2 | |
| | | hh:mm:ss. | hh:mm:ss} | | |
| W2 | P5 | Start up the IS at time: | {Time = | HA1-2 | |
| | | hh:mm:ss. | hh:mm:ss} | | |
| W3 | P5 | Start up the IS at time: | {Time = | HA-2 | |
| | | hh:mm:ss. | hh:mm:ss} | | |
| W4 | P5 | Start up the IS at time: | {Time = | CA-2 | |
| | | hh:mm:ss. | hh:mm:ss} | | |
| W5 | P5 | Start up the IS at time: | {Time = | FA-2 | |
| | | hh:mm:ss. | hh:mm:ss} | | |
| W6 | P5 | Start up the IS at time: | {Time = | M-2 | |
| | | hh:mm:ss. | hh:mm:ss} | | |
| T1 | P8 | This triggers M-1 to start | I#DOHA=Y | M-1 | |
| | | up | | | |
| T2 | P8 | Start up M-1 - Initiated | I#SMP=Y | M-1 | |
| | | by MS-1 | | | |
| T3 | P8 | Start up M-1 - Initiated | I#VMP=Y | M-1 | |
| | | by MV-1 | | | |
| T4 | P8 | Start up M-1 - Initiated | I#PP=Y | M-1 | |
| | | by MPP-1 | | | |
| T5 | P8 | If this Parameter is set, | I#M1DO=Y | M-1 | |
| | | start up IS#M-1. | | | |

The M-2 IS mentioned above is a probing IS that does a quick health check-up on the client shortly after M-1 was started up and did not identify an SHE. M-2 first just asks if the client is OK. If not, the client is asked what the problem is. If the client answers “OK”, then the system carries out the quick health checklist on the client. If any issue is identified, then control is sent to M-1. This IS can be activated by M-1 to start some time, such as 10 minutes, after M-1 finished.

The system can have specific checklists for determining if the client is experiencing a particular SHE. These checklists can be initiated by M-1 and are described further below.

Tables 23 and 24 are an exemplary IS table for M-2.

TABLE 23

| | | | |
|---|---|---|---|
| IS# | M-2 | TMT-IS Action | <CALL IS#LOS-1/ |
| | | | IU#600> |
| T-InterruptionMax | 300 | URW-IS Action | <CALL IS#LOS-1/ |
| | | | IU#700> |
| RMD-IS | 60 | NVI-IS Action | <CALL IS#LOS-1/ |
| | | | IU#800> |
| S-Time | | NUI-IS Action | <CALL IS#LOS-1/ |
| | | | IU#800> |

TABLE 24

| | | Decision Statement | | | | RMD-IU |
|---|---|---|---|---|---|---|
| IU # | Output Text String | Condition | Action | IU Grp | IMP# | (secs) |
| 10 | John, I'm just | Good | 200 | | | |
| | checking to see | Not Good | 50 | | | |
| | how you are - Are | | | | | |
| | you good or not | | | | | |
| | good? | | | | | |
| 50 | What is the | Pain | <S “Y”> | | PA | |
| | probelm? | Illness | <S “Y”> | | IL | |
| | | Weak | <S “Y”> | | WE | |
| | | Numbness | <S “Y”> | | NU | |
| | | Discomfort | <S “Y”> | | DI | |
| | | Breathing | <S “Y”> | | BR1 | |
| | | Fell | <S “Y”> | | FA | |
| | | Trouble | <S “Y”> | | TW1 | |
| | | Walking | | | | |
| | | Loss of Balance | <S “Y”> | | LBA | |
| | | Loss of | <S “Y”> | | LCO | |
| | | Coordination | | | | |
| | | Chest | <S “Y”> | | CH | |
| | | Heart | <S “Y”> | | HE | |
| | | Can't Move | <S “Y”> | | CM1 | |
| | | Can't Walk | <S “Y”> | | CM2 | |
| | | Feel Strange | <S “Y”> | | FS1 | |
| | | Feel Funny | <S “Y”> | | FS2 | |
| | | Something | <S “Y”> | | FS3 | |
| | | Wrong | | | | |
| | | Don't Feel | <S “Y”> | | FS4 | |
| | | Right | | | | |
| | | Nauseous | <S “Y”> | | NAU | |
| | | Dizzy | <S “Y”> | | DIZ | |
| | | Lightheaded | <S “Y”> | | LH | |
| | | Cold Sweat | <S “Y”> | | CS | |
| | | Droopy Face | <S “Y”> | | DF1 | |
| | | Droopy Mouth | <S “Y”> | | DF1 | |
| | | Headache | <S “Y”> | | PA | |
| | | <Other> | 200 | | | |
| 60 | <COMMENT: If | | <R1SAVE | | M1DO | |
| | the person says | | “1”> \|\| | | | |
| | any one of the | | <S “Y”> \|\| | | | |
| | above, control | | <END> | | | |
| | goes to IS# M-1 | | | | | |
| | for health situation | | | | | |
| | analysis.> \|\| | | | | | |
| | <NO OTS> | | | | | |
| 200 | <NRR>That's | | <END> | | | |
| | great. That's all for | | | | | |
| | now. Call out if | | | | | |
| | you suddenly don't | | | | | |
| | feel well. Or just | | | | | |
| | push the | | | | | |
| | Emergency Button | | | | | |
| | if it is an | | | | | |
| | Emergency. | | | | | |

Tables 25 and 26 show exemplary IS definition table for a physiological parameter IS.

TABLE 25

| | | | |
|---|---|---|---|
| IS# | MPP-1 | TMT-IS Action | <CALL IS#LOS-1/ |
| | | | IU#600> |
| T-InterruptionMax | 180 | URW-IS Action | <CALL IS#LOS-1/ |
| | | | IU#700> |
| RMD-IS | 60 | NVI-IS Action | <CALL IS#LOS-1/ |
| | | | IU#800> |
| S-Time | | NUI-IS Action | <CALL IS#LOS-1/ |
| | | | IU#800> |

TABLE 26

| | | Decision Statement | | | | RMD-IU |
|---|---|---|---|---|---|---|
| IU # | Output Text String | Condition | Action | IU Grp | IMP# | (secs) |
| 10 | <NO OTS> | P#HRL1=Y | <C IS#CA-1> | | | |
| | | P#RRN1=Y | <C IS#CA-1> | | | |
| | | P#ECN1=Y | <C IS#CA-1> | | | |
| | | P#BOL1=Y | <C IS#CA-1> | | | |
| | | <Other> | 30 | | | |
| 30 | <NO OTS> | | <S “Y”> | | PP | |
| 40 | <COMMENT: Control | | <END> | | | |
| | is sent to IS# M-1.> \|\| | | | | | |
| | <NO OTS> | | | | | |

Tables 27 and 28 show exemplary IS definition table for a sound parameter IS.

TABLE 27

| | | | |
|---|---|---|---|
| IS# | MS-1 | TMT-IS Action | <CALL IS#LOS-1/ |
| | | | IU#600> |
| T-InterruptionMax | 600 | URW-IS Action | <CALL IS#LOS-1/ |
| | | | IU#700> |
| RMD-IS | 60 | NVI-IS Action | <CALL IS#LOS-1/ |
| | | | IU#800> |
| S-Time | | NUI-IS Action | <CALL IS#LOS-1/ |
| | | | IU#800> |

TABLE 28

| | | Decision Statement | | | | RMD-IU |
|---|---|---|---|---|---|---|
| IU # | Output Text String | Condition | Action | IU Grp | IMP# | (secs) |
| 100 | <NO OTS> | S#PAS1=Y | 102 | | | |
| | | S#PAS1><Y | 110 | | | |
| 102 | I have detected cries of | Yes | <S> \|\| 123 | | PSVY | |
| | pain. | No | <S> \|\| 106 | | PSVY | |
| | Is there a problem? | | | | | |
| 106 | <NRR> | | <END> | | | |
| | Ok, I was mistaken. | | | | | |
| | Carry on. | | | | | |
| 110 | <NO OTS> | S#PAS2=Y | 112 | | | |
| | | S#PAS2><Y | 120 | | | |
| 111 | I have detected you | Yes | <S> \|\| 123 | | PSVY | |
| | saying “ouch”. | No | <S> \|\| 116 | | PSVY | |
| | Is there a problem? | | | | | |
| 112 | <NRR> | | <END> | | | |
| | Ok, I was mistaken. | | | | | |
| | Carry on. | | | | | |
| 120 | <NO OTS> | S#FAS1=Y | 121 | | | |
| | | S#FAS1><Y | 130 | | | |
| 121 | I have detected a falling | Yes | <S> \|\| 123 | | FSVY | |
| | sound. | No | <S> \|\| 122 | | FSVY | |
| | Did you just fall? | | | | | |
| 122 | <NRR> | | <END> | | | |
| | Ok, I was mistaken. | | | | | |
| | Carry on. | | | | | |
| 123 | <NO OTS> | | <S “Y”> | | SMP | |
| 124 | <COMMENT: Control | | <END> | | | |
| | is sent to IS# M-1 for | | | | | |
| | further probing.> \|\| | | | | | |
| | <NO OTS> | | | | | |
| 130 | <NO OTS> | S#EMK=Y | 132 | | | |
| | | S#EMK><Y | 140 | | | |
| 132 | I have detected you | No | 134 | | | 20 |
| | knocking the | S#KS2=Y | 138 | | | |
| | Emergency code. If this | TMT | 137 | | | |
| | is not the case, verbally | <Other> | 137 | | | |
| | say, “No”. If you are | | | | | |
| | trying to communicate | | | | | |
| | with me by making | | | | | |
| | knocking sounds, knock | | | | | |
| | 2 times. | | | | | |
| 134 | <NRR> | | <END> | | | |
| | Sorry. Carry on. | | | | | |
| 137 | <NRR> | | <S “Y”> \|\| | | EMC | |
| | I didn't hear 2 knocks | | <END> | | | |
| | from you. I am going to | | | | | |
| | call ERD as a | | | | | |
| | precaution. | | | | | |
| 138 | <NRR> | | <S “Y”> \|\| | | EM4 | |
| | OK, I am calling ERD | | <END> | | | |
| | to inform them that you | | | | | |
| | are in an Emergency | | | | | |
| | situation and that you | | | | | |
| | can't speak. | | | | | |
| 140 | <NO OTS> | S#EMY=Y | 142 | | | |
| | | S#EMY><Y | <END> | | | |
| 142 | I have detected you | No | 144 | | | 20 |
| | yelping the Emergency | S#YS2=Y | 148 | | | |
| | code. If this is not the | TMT | 147 | | | |
| | case, verbally say, | <Other> | 147 | | | |
| | “No”. If you are trying | | | | | |
| | to communicate with | | | | | |
| | me by making yelping | | | | | |
| | sounds, yelp 2 times. | | | | | |
| 144 | <NRR> | | <END> | | | |
| | Sorry. Carry on. | | | | | |
| 147 | <NRR> | | <S “Y”> \|\| | | EMC | |
| | I didn't hear 2 yelps | | <END> | | | |
| | from you. I am going to | | | | | |
| | call ERD as a | | | | | |
| | precaution. | | | | | |
| 148 | <NRR> | | <S “Y”> \|\| | | EM4 | |
| | OK, I am calling ERD | | <END> | | | |
| | to inform them that you | | | | | |
| | are in an Emergency | | | | | |
| | situation and that you | | | | | |
| | can't speak. | | | | | |

Tables 29 and 30 show exemplary IS definition table for a video parameter IS.

TABLE 29

| | | | |
|---|---|---|---|
| IS# | MV-1 | TMT-IS Action | <CALL IS#LOS-1/ |
| | | | IU#600> |
| T-InterruptionMax | 600 | URW-IS Action | <CALL IS#LOS-1/ |
| | | | IU#700> |
| RMD-IS | 60 | NVI-IS Action | <CALL IS#LOS-1/ |
| | | | IU#800> |
| S-Time | | NUI-IS Action | <CALL IS#LOS-1/ |
| | | | IU#800> |

TABLE 30

| | | Decision Statement | | | | RMD-IU |
|---|---|---|---|---|---|---|
| IU # | Output Text String | Condition | Action | IU Grp | IMP# | (secs) |
| 100 | <NO OTS> | V#FAV=Y | 102 | | | |
| | | V#FAV><Y | 110 | | | |
| 102 | I have detected you | Yes | <S> \|\| 118 | | FSVY | |
| | falling. | No | <S> \|\| 106 | | FSVY | |
| | Is this true? | | | | | |
| 106 | <NRR> | | <END> | | | |
| | Ok, I was mistaken. | | | | | |
| | Carry on. | | | | | |
| 110 | <NO OTS> | V#TWV=Y | 112 | | | |
| | | V#TWV><Y | 120 | | | |
| 112 | I have detected you | Yes | <S> \|\| 118 | | TW1 | |
| | stumbling while | No | <S> \|\| 116 | | TW1 | |
| | walking. Is this true? | | | | | |
| 116 | <NRR> | | <END> | | | |
| | Ok, I was mistaken. | | | | | |
| | Carry on. | | | | | |
| 118 | <NO OTS> | | <S “Y”> | | VMP | |
| 119 | <COMMENT: Control | | <END> | | | |
| | is sent to IS# M-1 for | | | | | |
| | further probing.> \|\| | | | | | |
| | <NO OTS> | | | | | |
| 120 | <NO OTS> | V#EMW=Y | 122 | | | |
| | | V#EMW><Y | 130 | | | |
| 122 | I have detected you | No | 124 | | | 20 |
| | waving your arm to | V#AW1=Y | 128 | | | |
| | signal “Emergency” If | TMT | 127 | | | |
| | this is not the case, | <Other> | 127 | | | |
| | verbally say, “No”. If | | | | | |
| | you are trying to | | | | | |
| | communicate with me, | | | | | |
| | wave your arm again. | | | | | |
| 124 | <NRR> | | <END> | | | |
| | Sorry. Carry on. | | | | | |
| 127 | <NRR> | | <S “Y”> \|\| | | EMC | |
| | I didn't see you wave | | <END> | | | |
| | your arm. I am going to | | | | | |
| | call ERD as a | | | | | |
| | precaution. | | | | | |
| 128 | <NRR> | | <S “Y”> \|\| | | EM4 | |
| | OK, I am calling ERD | | <END> | | | |
| | to inform them that you | | | | | |
| | are in an Emergency | | | | | |
| | situation and that you | | | | | |
| | can't speak. | | | | | |
| 130 | <NO OTS> | V#EML=Y | 132 | | | |
| | | V#EML><Y | <END> | | | |
| 132 | I have detected you | No | 134 | | | 20 |
| | lifting your leg to signal | V#LR1=Y | 138 | | | |
| | an Emergency. If this is | TMT | 137 | | | |
| | not the case, verbally | <Other> | 137 | | | |
| | say, “No”. If you are | | | | | |
| | trying to communicate | | | | | |
| | with me, lift your leg | | | | | |
| | again. | | | | | |
| 134 | <NRR> | | <END> | | | |
| | Sorry. Carry on. | | | | | |
| 137 | <NRR> | | <S “Y”> \|\| | | EMC | |
| | I didn't see you lift your | | <END> | | | |
| | leg. I am going to call | | | | | |
| | ERD as a precaution. | | | | | |
| 138 | <NRR> | | <S “Y”> \|\| | | EM4 | |
| | OK, I am calling ERD | | <END> | | | |
| | to inform them that you | | | | | |
| | are in an Emergency | | | | | |
| | situation and that you | | | | | |
| | can't speak. | | | | | |

An S-1 checklist checks if the client is experiencing the early warning signs of a stroke or an actual stoke.

a) Check if have sudden numbness/weakness on one side of body—arm, leg, face?
If answer “Yes” verbally, go to c)
If answer “Yes” non-verbally (vocal sound, hitting sound, waving), due to trouble speaking→emergency detected—Stroke
If answer “No”, go to b)
If answer “Not sure”, go to b)
If confused, do “Loss of Understanding” Test; if fail→emergency detected

b) Perform the “Arm Drift Test”. Ask person to put both arms straight out, and to hold them there for as long as they can. When one or both come down, ask if one arm came down sooner than the other.
If answer “Yes” verbally, go to c)
If answer “Yes” non-verbally (vocal sound, hitting sound, waving), due to trouble speaking→emergency detected (ED)—Stroke
If answer “No” or “Not sure” verbally, activate S-2
If answer “No” or “Not sure” non-verbally→emergency detected

c) Perform the “Droopy Face” Test. Ask the person to go in front of a mirror and to smile. Ask him/her, “Do you have a problem smiling?” and “Does your face/mouth droop on one side?”
If answer is “Yes”→ED—Stroke
If answer is “No”, activate S-2

Tables 31 and 32 show IS Definitions for S-1.

TABLE 31

| | | | |
|---|---|---|---|
| IS# | S-1 | TMT-IS Action | <CALL IS#LOS-1/ |
| | | | IU#600> |
| T-InterruptionMax | 600 | URW-IS Action | <CALL IS#LOS-1/ |
| | | | IU#700> |
| RMD-IS | 60 | NVI-IS Action | <CALL IS#LOS-1/ |
| | | | IU#800> |
| S-Time | | NUI-IS Action | <CALL IS#LOS-1/ |
| | | | IU#800> |

TABLE 32

| | | | | | | RMD- |
|---|---|---|---|---|---|---|
| | | Decision Statement | | IU | | IU |
| IU # | Output Text String | Condition | Action | Grp | IMP# | (secs) |
| 5 | <NO OTS> | | <SAVE “Y”> | | DOS | |
| 10 | <NRR> | | <GOTO IU#15> | | | 1 |
| | John, you may be | | | | | |
| | experiencing the EWSs of a | | | | | |
| | health problem. I need to | | | | | |
| | ask you a few questions to | | | | | |
| | help evaluate the situation. | | | | | |
| 15 | Do you have sudden | Yes | <SAVE> \|\| 30 | | NU | |
| | numbness? | No | <SAVE> \|\| 20 | | NU | |
| 20 | Do you have sudden | Yes | <SAVE>\|\|40 | | WE | |
| | weakness? | No | <SAVE>\|\|50 | | WE | |
| 30 | Where is it located? | Arm | <SAVE>\|\|35 | | NUL | |
| | | Leg | <SAVE>\|\|35 | | NUL | |
| | | Face | <SAVE>\|\|35 | | NUL | |
| | | Other | 50 | | NUL | |
| 35 | Is it on one side of the | Yes | 37 | | | |
| | body? | No | <SAVE “Both” | | N1S | |
| | | | \|\|50 | | | |
| | | Not sure | <SAVE | | N1S | |
| | | | “Unsure”\|\|50 | | | |
| 37 | Right or left side? | Right | <SAVE>\|\|500 | | NSI | |
| | | Left | <SAVE>\|\|500 | | NSI | |
| 40 | Where is it located? | Arm | <SAVE>\|\|45 | | WEL | |
| | | Leg | <SAVE>\|\|45 | | WEL | |
| | | Face | <SAVE>\|\|45 | | WEL | |
| | | Other | 50 | | WEL | |
| 45 | Is it on one side of the | Yes | <SAVE>\|\|47 | | W1S | |
| | body? | No | <SAVE | | W1S | |
| | | | “Both”>\|\|50 | | | |
| | | Not sure | <SAVE | | W1S | |
| | | | “Unsure”\|\|50 | | | |
| 47 | Right or left side? | Right | <SAVE>\|\|500 | | WSI | |
| | | Left | <SAVE>\|\|500 | | WSI | |
| 50 | I would like you to do a | Down | 60 | | | |
| | quick test, called the “Arm | | | | | |
| | Drift” Test. While standing, | | | | | |
| | please put both arms | | | | | |
| | straight out in front of you. | | | | | |
| | Now try to hold them there | | | | | |
| | for as long as you can. Say | | | | | |
| | “down” when both arms or | | | | | |
| | one arm comes down a few | | | | | |
| | inches. | | | | | |
| 60 | Did one arm come down | Yes | <SAVE>\|\|65 | | AD1 | |
| | faster than the other? | No | <SAVE >\|\|560 | | AD1 | |
| 65 | Right or left arm? | Right | <SAVE>\|\|500 | | AD2 | |
| | | Left | <SAVE>\|\|500 | | AD2 | |
| 500 | I want you to carry out the | Ready | 510 | | | |
| | “Smile” Test. Please go in | | | | | |
| | front of a large mirror. Say | | | | | |
| | “ready” when you are there. | | | | | |
| 510 | Now I want you to look | Yes | <SAVE>\|\|550 | | ST1 | |
| | closely at your face and try | No | <SAVE>\|\|520 | | ST1 | |
| | to make a big smile. Do you | | | | | |
| | have trouble making a | | | | | |
| | smile? | | | | | |
| 520 | Does your face or mouth | Yes | 525 | | | |
| | look like it's drooping? | No | <SAVE>\|\|560 | | ST2 | |
| 525 | Does it droop on one side? | Yes | 530 | | | |
| | | No | 560 | | | |
| 530 | Right or left side? | Right | <SAVE>\|\|550 | | ST3 | |
| | | Left | <SAVE>\|\|550 | | ST3 | |
| 550 | <COMMENT Stroke | | <END> | | | |
| | Emergency Detection will | | | | | |
| | be activated. Another IS | | | | | |
| | will start communicating | | | | | |
| | with the person.>\|\| | | | | | |
| | <NO OTS> | | | | | |
| 560 | <NRR> | | <WAIT-600s | | | |
| | That's all for now. I will | | IS#S-2> \|\| | | | |
| | check in with you in 5 | | <RETURN> | | | |
| | minutes. | | | | | |
| | I suggest that you sit down | | | | | |
| | for a few minutes. | | | | | |
| | If at any time you feel that | | | | | |
| | the situation is an | | | | | |
| | emergency, press the button | | | | | |
| | on the EB device, or call | | | | | |
| | out to me for help. | | | | | |

S-2 is a follow up IS that can be carried out shortly after S-1 has finished its analysis and has not found evidence of a Stroke. The purpose of S-2 is to ensure that the client did not develop signs of stroke after S-1 finished its analysis. S-2 either performs the same procedure as S-1, or it may just do a quick check.

Tables 33 and 34 show IS Definitions for S-2.

TABLE 33

| | | | |
|---|---|---|---|
| IS# | S-2 | TMT-IS Action | <CALL IS#LOS-1/ |
| | | | IU#600> |
| T-InterruptionMax | 180 | URW-IS Action | <CALL IS#LOS-1/ |
| | | | IU#700> |
| RMD-IS | 60 | NVI-IS Action | <CALL IS#LOS-1/ |
| | | | IU#800> |
| S-Time | | NUI-IS Action | <CALL IS#LOS-1/ |
| | | | IU#800> |

TABLE 34

| | | Decision Statement | | IU | | RMD-IU |
|---|---|---|---|---|---|---|
| IU # | Output Text String | Condition | Action | Grp | IMP# | (secs) |
| 5 | <NO OTS> | | <SAVE “Y”> | | DOS | |
| 10 | <NRR> | | <GOTO IU#15> | | | 1 |
| | John, I'm back to see how | | | | | |
| | you are doing. I have a few | | | | | |
| | questions for you. | | | | | |
| 15 | Do you have sudden | Yes | <SAVE> \|\| 30 | | NU | |
| | numbness? | No | <SAVE> \|\| 20 | | NU | |
| 20 | Do you have sudden | Yes | <SAVE>\|\|40 | | WE | — |
| | weakness? | No | <SAVE>\|\|50 | | WE | |
| 30 | Where is it located? | Arm | <SAVE>\|\|35 | | NUL | |
| | | Leg | <SAVE>\|\|35 | | NUL | |
| | | Face | <SAVE>\|\|35 | | NUL | |
| | | Other | 50 | | NUL | |
| 35 | Is it on one side of the | Yes | 37 | | | |
| | body? | No | <SAVE “Both” | | N1S | |
| | | | \|\|50 | | | |
| | | Not sure | <SAVE | | N1S | |
| | | | “Unsure”\|\|50 | | | |
| 37 | Right or left side? | Right | <SAVE>\|\|500 | | NSI | |
| | | Left | <SAVE>\|\|500 | | NSI | |
| 40 | Where is it located? | Arm | <SAVE>\|\|45 | | WEL | |
| | | Leg | <SAVE>\|\|45 | | WEL | |
| | | Face | <SAVE>\|\|45 | | WEL | |
| | | Other | 50 | | WEL | |
| 45 | Is it on one side of the | Yes | <SAVE>\|\|47 | | W1S | |
| | body? | No | <SAVE | | W1S | |
| | | | “Both”>\|\|50 | | | |
| | | Not sure | <SAVE | | W1S | |
| | | | “Unsure”\|\|50 | | | |
| 47 | Right or left side? | Right | <SAVE>\|\|500 | | WSI | |
| | | Left | <SAVE>\|\|500 | | WSI | |
| 50 | I would like you to do a | Down | 60 | | | |
| | quick test, called the “Arm | | | | | |
| | Drift” Test. While | | | | | |
| | standing, please put both | | | | | |
| | arms straight out in front of | | | | | |
| | you. Now try to hold them | | | | | |
| | there for as long as you | | | | | |
| | can. Say “down” when | | | | | |
| | both arms or one arm | | | | | |
| | comes down a few inches. | | | | | |
| 60 | Did one arm come down | Yes | <SAVE>\|\|65 | | AD1 | |
| | faster than the other? | No | <SAVE >\|\|560 | | AD1 | |
| 65 | Right or left arm? | Right | <SAVE>\|\|500 | | AD2 | |
| | | Left | <SAVE>\|\|500 | | AD2 | |
| 500 | I want you to carry out the | Ready | 510 | | | |
| | “Smile” Test. Please go in | | | | | |
| | front of a large mirror. Say | | | | | |
| | “ready” when you are | | | | | |
| | there. | | | | | |
| 510 | Now I want you to look | Yes | <SAVE>\|\|550 | | ST1 | |
| | closely at your face and try | No | <SAVE>\|\|520 | | ST1 | |
| | to make a big smile. Do | | | | | |
| | you have trouble making a | | | | | |
| | smile? | | | | | |
| 520 | Does your face or mouth | Yes | 525 | | | |
| | look like it's drooping? | No | <SAVE>\|\|560 | | ST2 | |
| 525 | Does it droop on one side? | Yes | 530 | | | |
| | | No | 560 | | | |
| 530 | Right or left side? | Right | <SAVE>\|\|550 | | ST3 | |
| | | Left | <SAVE>\|\|550 | | ST3 | |
| 550 | <COMMENT Stroke | | <END> | | | |
| | Emergency Detection will | | | | | |
| | be activated. Another IS | | | | | |
| | will start communicating | | | | | |
| | with the person.>\|\| | | | | | |
| | <NO OTS> | | | | | |
| 560 | <NRR> | | <END> | | | |
| | That's all for now. | | | | | |
| | If at any time you feel that | | | | | |
| | the situation is an | | | | | |
| | emergency, press the | | | | | |
| | button on the EB device, or | | | | | |
| | call out to me for help. | | | | | |

S-3 is a probing IS that is carried out when it has been detected that the client cannot speak, but can hear, and can communicate non-verbally (knocking on something, or making vocal sounds, or waving an arm, or lifting a leg). This Probing IS is also executed when it has been detected that the client has trouble speaking. Tables 35 and 36 show IS Definitions for S-2.

TABLE 35

| | | |
|---|---|---|
| IS# | S-3 | TMT-IS Action |
| T-InterruptionMax | 600 | URW-IS Action |
| RMD-IS | 60 | NVI-IS Action |
| S-Time | | NUI-IS Action |

TABLE 36

| | | | | | | RMD- |
|---|---|---|---|---|---|---|
| | | Decision Statement | | IU | | IU |
| IU # | Output Text String | Condition | Action | Grp | IMP# | (secs) |
| 5 | <NO OTS> | | <SAVE “Y”> | | DOS | |
| 7 | <NRR> | | 15 | | | |
| | I am going to ask you a | | | | | |
| | few questions. Please | | | | | |
| | knock or yelp once for | | | | | |
| | ‘Yes’, and knock or | | | | | |
| | yelp twice for ‘No’. If | | | | | |
| | at any time, you feel | | | | | |
| | that it is an Emergency, | | | | | |
| | knock or yelp twice, | | | | | |
| | pause, then knock or | | | | | |
| | yelp twice again.> | | | | | |
| 15 | Do you have sudden | (KS1=Y) OR | <S “Y”> \|\| 30 | | NU | |
| | numbness? | (YS1=Y) | | | | |
| | | (KS2=Y) OR | <S “N”> \|\| 20 | | NU | |
| | | (YS2=Y) | | | | |
| 20 | Do you have sudden | (KS1=Y) OR | <S “Y”>\|\|40 | | WE | |
| | weakness? | (YS1=Y) | | | WE | |
| | | (KS2=Y) OR | <S “N”>\|\|50 | | | |
| | | (YS2=Y) | | | | |
| 30 | Located in the Arm? | (KS1=Y) OR | <S “Arm”> | | NUL | |
| | | (YS1=Y) | 31 | | | |
| | | (KS2=Y) OR | | | | |
| | | (YS2=Y) | | | | |
| 31 | Located in the Leg? | (KS1=Y) OR | <S “Leg”> | | NUL | |
| | | (YS1=Y) | 32 | | | |
| | | (KS2=Y) OR | | | | |
| | | (YS2=Y) | | | | |
| 32 | Located in the Face? | (KS1=Y) OR | <S “Face”> | | NUL | |
| | | (YS1=Y) | 50 | | | |
| | | (KS2=Y) OR | | | | |
| | | (YS2=Y) | | | | |
| 35 | Is it on one side of the | (KS1=Y) OR | <S “Y”>\|\|500 | | N1S | |
| | body? | (YS1=Y) | | | | |
| | | (KS2=Y) OR | <S “Both” | | N1S | |
| | | (YS2=Y) | \|\|50 | | | |
| 37 | Located in the Arm? | (KS1=Y) OR | <S “Arm”> | | WEL | |
| | | (YS1=Y) | 31 | | | |
| | | (KS2=Y) OR | | | | |
| | | (YS2=Y) | | | | |
| 38 | Located in the Leg? | (KS1=Y) OR | <S “Leg”> | | WEL | |
| | | (YS1=Y) | 32 | | | |
| | | (KS2=Y) OR | | | | |
| | | (YS2=Y) | | | | |
| 39 | Located in the Face? | (KS1=Y) OR | <S “Face”> | | WEL | |
| | | (YS1=Y) | 50 | | | |
| | | (KS2=Y) OR | | | | |
| | | (YS2=Y) | | | | |
| 45 | Is it on one side of the | (KS1=Y) OR | <S “Y”>\|\|500 | | W1S | |
| | body? | (YS1=Y) | | | | |
| | | (KS2=Y) OR | <S “Both”> | | W1S | |
| | | (YS2=Y) | \|\|50 | | | |
| 50 | I would like you to do a | (KS1=Y) OR | 60 | | | |
| | quick test, called the | (YS1=Y) | | | | |
| | “Arm Drift” Test. | | | | | |
| | While standing, please | | | | | |
| | put both arms straight | | | | | |
| | out in front of you. | | | | | |
| | Now try to hold them | | | | | |
| | there for as long as you | | | | | |
| | can. Do a ‘Yes’ when | | | | | |
| | both arms or one arm | | | | | |
| | comes down a few | | | | | |
| | inches. | | | | | |
| 60 | Did one arm come | (KS1=Y) OR | <S “Y”>\|\|500 | | AD1 | |
| | down faster than the | (YS1=Y) | | | | |
| | other? | (KS2=Y) OR | <S “N”>\|\|560 | | AD1 | |
| | | (YS2=Y) | | | | |
| 500 | I want you to carry out | (KS1=Y) OR | 510 | | | |
| | the “Smile” Test. Please | (YS1=Y) | | | | |
| | go in front of a large | | | | | |
| | mirror. Do a ‘Yes’ | | | | | |
| | when you are ready. | | | | | |
| 510 | Now I want you to look | (KS1=Y) OR | <S “Y”>\|\|550 | | ST1 | |
| | closely at your face and | (YS1=Y) | | | | |
| | try to make a big smile. | (KS2=Y) OR | <S “N”>\|\|520 | | ST1 | |
| | Do you have trouble | (YS2=Y) | | | | |
| | making a smile? | | | | | |
| 520 | Does your face or | (KS1=Y) OR | <S “Y”>\|\|525 | | ST2 | |
| | mouth look like it's | (YS1=Y) | | | | |
| | drooping? | (KS2=Y) OR | <S “N”>\|\|560 | | ST2 | |
| | | (YS2=Y) | | | | |
| 525 | Does it droop on one | (KS1=Y) OR | <S “Y”>\|\|550 | | F1S | |
| | side? | (YS1=Y) | | | | |
| | | (KS2=Y) OR | <S “N”>\|\|560 | | F1S | |
| | | (YS2=Y) | | | | |
| 550 | <COMMENT Stroke | | <END> | | | |
| | Emergency Detection | | | | | |
| | will be activated. | | | | | |
| | Another IS will start | | | | | |
| | communicating with the | | | | | |
| | person.>\|\| | | | | | |
| | <NO OTS> | | | | | |
| 560 | <NO OTS> | | <RETURN> | | | |

HA-1 is a heart attack check IS that is activated by M-1, after M-1 has analyzed the information it received, plus the information it gathered, and concluded that the situation could be a possible heart attack. The HA-1 can be initiated by a low or high heart rate. The purpose of HA-1 is to check if the client is showing the early warning signs of a heart attack, or is experiencing a heart attack. It does this by carrying out verbal interaction with the client. It asks the client a few key questions that are associated with heart attack. If HA-1 identified heart attack symptoms in the client, but the symptoms have not lasted for at least 5 minutes, then it activates HA-1-2 to start up later, such as 4 minutes later. HA-1 then ends. If HA-1 does not identify heart attack-based SHE, it then activates HA-2 to start up later, such as 10 minutes later, as a follow-up. HA-1 then ends.

The heart attack HA-1 IS can include the following inquiry.

a) Check if have pain in the center of the chest that has been there steady, or that started, went away, and then came back.
If No, go to c)
If Yes, go to b)

b) Has it lasted for more than 5 minutes.
If Yes→ED—Heart Attack
If No, activate HA-1-2 to start in 4 minutes

c) Check if have discomfort in the center of the chest that has been there steady, or that started, went away, and then came back—pressure, fullness, squeezing.
If No, activate HA-2 to start in 10 minutes.
If Yes, go to d)

d) Has it lasted for more than 5 minutes.
If Yes→ED—Heart Attack
If No, activate HA-1-2 to start in 4 minutes

Tables 37 and 38 show IS Definitions for HA-1.

TABLE 37

| | | | |
|---|---|---|---|
| IS# | HA-1 | TMT-IS Action | <CALL IS#LOS-1/ |
| | | | IU#600> |
| T-InterruptionMax | 600 | URW-IS Action | <CALL IS#LOS-1/ |
| | | | IU#700> |
| RMD-IS | 60 | NVI-IS Action | <CALL IS#LOS-1/ |
| | | | IU#800> |
| S-Time | | NUI-IS Action | <CALL IS#LOS-1/ |
| | | | IU#800> |

TABLE 38

| | | Decision Statement | | IU | | RMD-IU |
|---|---|---|---|---|---|---|
| IU # | Output Text String | Condition | Action | Grp | IMP# | (secs) |
| 5 | <NO OTS> | | <SAVE “Y”> | | DOHA | |
| 10 | <NRR> | | <GOTO IU#20> | | | |
| | John, you may be | | | | | |
| | experiencing the EWSs of | | | | | |
| | a health problem. I need | | | | | |
| | to ask you a few questions | | | | | |
| | to help evaluate the | | | | | |
| | situation. | | | | | |
| 20 | First question: Do you | Yes | <SAVE>\|\|40 | | PCH | |
| | have pain in the chest? | No | <SAVE>\|\|30 | | PCH | |
| 30 | Do you have discomfort | Yes | <SAVE>\|\|100 | | DCH | |
| | in the chest? | No | <SAVE>\|\|200 | | DCH | |
| 40 | Is the pain coming from | Yes | <SAVE>\|\|45 | | PCC | |
| | the center of the chest? | No | <SAVE>\|\|200 | | PCC | |
| 45 | Has the pain been fairly | Steady | <SAVE>\|\|50 | | PS | |
| | steady or did it come and | Not steady | <SAVE>\|\|50 | | PS | |
| | go? | | | | | |
| 50 | Has it lasted for more than | Yes | <SAVE>\|\|550 | | PG5 | |
| | 5 minutes? | No | <SAVE>\|\|160 | | PG5 | |
| 100 | Is the discomfort coming | Yes | <SAVE>\|\|120 | | DCC | |
| | from the center of the | No | <SAVE>\|\|200 | | DCC | |
| | chest? | | | | | |
| 120 | What kind of discomfort | Pressure | <SAVE>\|\|140 | | DT | |
| | is it?: pressure, squeezing, | Squeezing | <SAVE>\|\|140 | | DT | |
| | or fullness? | Fullness | <SAVE>\|\|140 | | DT | |
| | | <Other> | 140 | | | |
| 140 | Has the discomfort been | Steady | <SAVE>\|\|150 | | DS | |
| | fairly steady or did it | Not steady | <SAVE>\|\|150 | | DS | |
| | come and go? | | | | | |
| 150 | Has it lasted for more than | Yes | <SAVE>\|\|550 | | DG5 | |
| | 5 minutes? | No | <SAVE>\|\|160 | | DG5 | |
| 160 | I will check back with you | | <R3SAVE “1”> | | | |
| | in 4 minutes. | | \|\|<WAIT-240s | | | |
| | If at any time you feel that | | IS#HA-1-2> \|\| | | | |
| | the situation is an | | <RETURN> | | | |
| | emergency, press the EB, | | | | | |
| | or call out to me for help. | | | | | |
| 200 | I will check back with you | | <WAIT-600s | | | |
| | in 10 minutes. | | IS#HA-2> \|\| | | | |
| | If at any time you feel that | | <RETURN> | | | |
| | the situation is an | | | | | |
| | emergency, press the EB, | | | | | |
| | or call out to me for help. | | | | | |
| 550 | <COMMENT Heart | | <END> | | | |
| | Attack Emergency | | | | | |
| | Detection will be | | | | | |
| | activated. | | | | | |
| | Another IS will start | | | | | |
| | communicating with the | | | | | |
| | person.>\|\| | | | | | |
| | <NO OTS> | | | | | |

HA-1-2 is started up by HA-1 (or HA-2), when required. If HA-1 (or HA-2) identified heart attack symptoms in the client, but the symptoms have not lasted for at least 5 minutes, then it activates HA-1-2 to start up later, such as 4 minutes later. The purpose of HA-1-2 is to check if the client's heart attack-related symptoms are still there. If they are, it identifies a heart attack related SHE. If the symptoms are no longer there, and HA-1-2 was activated by HA-1, it then activates HA-2 to start up 10 minutes later, as a follow-up. HA-1-2 then ends.

Tables 39 and 40 show IS Definitions for HA-1-2.

TABLE 39

| | | | |
|---|---|---|---|
| IS# | HA-1-2 | TMT-IS Action | <CALL IS#LOS-1/ |
| | | | IU#600> |
| T-InterruptionMax | 600 | URW-IS Action | <CALL IS#LOS-1/ |
| | | | IU#700> |
| RMD-IS | 60 | NVI-IS Action | <CALL IS#LOS-1/ |
| | | | IU#800> |
| S-Time | | NUI-IS Action | <CALL IS#LOS-1/ |
| | | | IU#800> |

TABLE 40

| | | Decision Statement | | | | RMD-IU |
|---|---|---|---|---|---|---|
| IU # | Output Text String | Condition | Action | IU Grp | IMP# | (secs) |
| 5 | <NO OTS> | | <SAVE “Y”> | | DOHA | |
| 10 | <NRR> | | 20 | | | |
| | All right, John, I'm | | | | | |
| | back to check how | | | | | |
| | you are doing. | | | | | |
| 20 | Do you have the | Yes | <SAVE> \|\| 22 | | PCC | |
| | pain in the center | No | <SAVE> \|\| 25 | | PCC | |
| | of your chest? | | | | | |
| 22 | <NO OTS> | | <S “Y”> \|\| 550 | | PG5 | |
| 25 | Do you have | Yes | <SAVE> \|\| 27 | | DCC | |
| | discomfort in the | No | <SAVE> \|\| 30 | | DCC | |
| | center of your | | | | | |
| | chest? | | | | | |
| 27 | <NO OTS> | | <S “Y”> \|\| 550 | | DG5 | |
| 30 | <COMMENT If | REG3=1 | 40 | | | |
| | came from HA-1, | REG3=2 | 260 | | | |
| | go to HA-2; if | | | | | |
| | came from HA-2, | | | | | |
| | End.> \|\| | | | | | |
| | <NO OTS> | | | | | |
| 40 | <NRR> | | <WAIT-600s | | | |
| | I will check in on | | IS#HA-2> | | | |
| | you in 10 minutes. | | <END> | | | |
| 260 | <NRR> | | <END> | | | |
| | I am finished | | | | | |
| | checking in with | | | | | |
| | you at this time. | | | | | |
| | If at any time you | | | | | |
| | do not feel well, | | | | | |
| | just call out for | | | | | |
| | help. If it is very | | | | | |
| | severe, push the | | | | | |
| | Emergency Button. | | | | | |
| 550 | <COMMENT Heart | | <END> | | | |
| | Attack Emergency | | | | | |
| | Detection will be | | | | | |
| | activated. | | | | | |
| | Another IS will start | | | | | |
| | communicating with | | | | | |
| | the person.>\|\| | | | | | |
| | <NO OTS> | | | | | |

HA-2 is a follow up IS carried out shortly after HA-1, or HA-1-2, has finished its analysis and has not found evidence of a Heart Attack. The purpose of HA-2 is to ensure that the client did not develop signs of a heart attack after HA-1 (HA-1-2) finished its analysis. HA-2 either performs the same procedure as HA-1, or it may just do a quick check.

HA-2 can be in the form of the following query.

a) Check if client has pain in the center of the chest that has been there steady, or that started, went away, and then came back (since the last check 10 minutes ago).
If No, go to c)
If Yes, go to b)

b) Has it lasted for more than 5 minutes?
If Yes→ED—Heart Attack
If No, activate HA-1-2 to start in 4 minutes

c) Check if have discomfort in the center of the chest that has been there steady, or that started, went away, and then came back—pressure, fullness, squeezing (since the last check 10 minutes ago).
If No, activate HA-2 to start in 10 minutes.
If Yes, go to d)

d) Has it lasted for more than 5 minutes?
If Yes→ED—Heart Attack
If No, activate HA-1-2 to start in 4 minutes

Tables 41 and 42 show IS Definitions for HA-2.

TABLE 41

| | | | |
|---|---|---|---|
| IS# | HA-2 | TMT-IS Action | <CALL IS#LOS-1/ |
| | | | IU#600> |
| T-InterruptionMax | 600 | URW-IS Action | <CALL IS#LOS-1/ |
| | | | IU#700> |
| RMD-IS | 60 | NVI-IS Action | <CALL IS#LOS-1/ |
| | | | IU#800> |
| S-Time | | NUI-IS Action | <CALL IS#LOS-1/ |
| | | | IU#800> |

TABLE 42

| | Output Text | Decision Statement | | | | RMD-IU |
|---|---|---|---|---|---|---|
| IU # | String | Condition | Action | IU Grp | IMP# | (secs) |
| 5 | <NO OTS> | | <SAVE “Y”> | | DOHA | |
| 10 | <NRR> | | 20 | | | |
| | All right, John, | | | | | |
| | I'm back to | | | | | |
| | check how you | | | | | |
| | are doing. | | | | | |
| 20 | Do you have | Yes | <SAVE>\|\|40 | | PCH | |
| | pain your chest, | No | <SAVE>\|\|30 | | PCH | |
| | now, or since the | | | | | |
| | last time I talked | | | | | |
| | to you? | | | | | |
| 30 | Do you have | Yes | <SAVE>\|\|100 | | DCH | |
| | discomfort in | No | <SAVE>\|\|60 | | DCH | |
| | your chest, now, | | | | | |
| | or since the last | | | | | |
| | time I talked to | | | | | |
| | you? | | | | | |
| 40 | Is the pain | Yes | <SAVE>\|\|45 | | PCC | |
| | coming from the | No | <SAVE>\|\|260 | | PCC | |
| | center of the | | | | | |
| | chest? | | | | | |
| 45 | Has the pain | Steady | <SAVE>\|\|50 | | PS | |
| | been fairly | Not steady | <SAVE>\|\|50 | | PS | |
| | steady or did it | | | | | |
| | come and go? | | | | | |
| 50 | Has it lasted for | Yes | <SAVE>\|\|550 | | PG5 | |
| | more than 5 | No | <SAVE>\|\|160 | | PG5 | |
| | minutes? | | | | | |
| 100 | Is the discomfort | Yes | <SAVE>\|\|120 | | DCC | |
| | coming from the | No | <SAVE>\|\|260 | | DCC | |
| | center of the | | | | | |
| | chest? | | | | | |
| 120 | What kind of | Pressure | <SAVE>\|\|140 | | DT | |
| | discomfort is it?: | Squeezing | <SAVE>\|\|140 | | DT | |
| | pressure, | Fullness | <SAVE>\|\|140 | | DT | |
| | squeezing, or | | | | | |
| | fullness? | | | | | |
| 140 | Has the | Steady | <SAVE>\|\|150 | | DS | |
| | discomfort been | Not steady | <SAVE>\|\|150 | | DS | |
| | fairly steady or | | | | | |
| | did it come and | | | | | |
| | go? | | | | | |
| 150 | Has it lasted for | Yes | <SAVE>\|\|550 | | DG5 | |
| | more than 5 | No | <SAVE>\|\|160 | | DG5 | |
| | minutes? | | | | | |
| 160 | I will check back | | <R3SAVE “2”> | | | |
| | with you in 4 | | \|\|<WAIT-240s | | | |
| | minutes. | | IS#HA-1-2> \|\| | | | |
| | If at any time | | <END> | | | |
| | you feel that the | | | | | |
| | situation is an | | | | | |
| | emergency, | | | | | |
| | press the EB, or | | | | | |
| | call out to me | | | | | |
| | for help. | | | | | |
| 260 | <NRR> | | <END> | | | |
| | That's all for | | | | | |
| | now. I cannot | | | | | |
| | detect any | | | | | |
| | sudden, new | | | | | |
| | health problems | | | | | |
| | at this time. | | | | | |
| | If at any time | | | | | |
| | you feel that the | | | | | |
| | situation is an | | | | | |
| | emergency, | | | | | |
| | press the EB, or | | | | | |
| | call out to me | | | | | |
| | for help. | | | | | |
| 550 | <COMMENT | | <END> | | | |
| | Heart Attack | | | | | |
| | Emergency | | | | | |
| | Detection will | | | | | |
| | be activated. | | | | | |
| | Another IS will | | | | | |
| | start | | | | | |
| | communicating | | | | | |
| | with the | | | | | |
| | person.>\|\| | | | | | |
| | <NO OTS> | | | | | |

A CA-1 IS is an IS activated by M-1, after M-1 has analyzed the information it received, plus the information it gathered, and concluded that the situation could be the possible early stages of cardiac arrest. The purpose of this CA-1 is to check if the client is showing the early warning signs of a cardiac arrest. It does this by carrying out verbal interaction with the client and asking the client a few key questions that are associated with the early warning signs of cardiac arrest. If CA-1 does not identify early stage cardiac arrest-based SHE, it then activates CA-2 to start up 10 minutes later, as a follow-up. CA-1 then ends.

The CA-1 query follows.

a) Ask person how he/she feels.
If Bad→ED
If No Verbal Response→ED
If Lack of Understanding→ED
If OK, go to b)

b) Ask person to quickly check equipment (simple things like checking for a loose connection).
If no equipment problems found, or not sure, go to c)
If equipment problems found, try to get person to fix
If fixed, and still poor PP, go to c)
If fixed, and poor PP goes away, End
If can't fix→ED—Equip
If taking too long,→ED—Equip

c) Activate CA-2 to start up in 5 minutes.

Tables 43 and 44 show IS Definitions for CA-1.

TABLE 43

| | | | |
|---|---|---|---|
| IS# | CA-1 | TMT-IS Action | <CALL IS#LOS-1/ |
| | | | IU#600> |
| T-InterruptionMax | 300 | URW-IS Action | <CALL IS#LOS-1/ |
| | | | IU#700> |
| RMD-IS | 60 | NVI-IS Action | <CALL IS#LOS-1/ |
| | | | IU#800> |
| S-Time | | NUI-IS Action | <CALL IS#LOS-1/ |
| | | | IU#800> |

TABLE 44

| | | | | | | RMD- |
|---|---|---|---|---|---|---|
| | | Decision Statement | | IU | | IU |
| IU # | Output Text String | Condition | Action | Grp | IMP# | (secs) |
| 5 | <NO OTS> | | <S “Y”> | | DOCA | |
| 10 | <NAME> | Good | <SAVE> \|\| 100 | | OK1 | |
| | <N>, I need to do a quick | Bad | <SAVE> \|\| 400 | | OK1 | |
| | health check on you. | In Between | <SAVE> \|\| 100 | | OK1 | |
| | Question: How do you | | | | | |
| | feel - Good, Bad, In | | | | | |
| | between? | | | | | |
| 100 | Could you do a quick | Done | 140 | | | 180 |
| | check of the connections | Help | 110 | | | |
| | on your on-person | TMT | 135 | | | |
| | monitoring devices. Call | | | | | |
| | out “Done” when you are | | | | | |
| | finished. Yell out “Help” | | | | | |
| | if you suddenly don't feel | | | | | |
| | well. | | | | | |
| 110 | <NRR> | | <SAVE | | EMCS | |
| | John, I will call for help | | “Yes”>\|\|END | | [Client | |
| | right now. | | | | asking for | |
| | | | | | Help] | |
| 135 | <NRR> | | <SAVE | | EQP1 | |
| | You seem to be having | | “Yes”>\|\|END | | [Possible | |
| | difficulties with the | | | | problem | |
| | equipment. I will call the | | | | with | |
| | Control Center and get | | | | equipment] | |
| | them to help you with the | | | | | |
| | situation. | | | | | |
| 140 | <COMMENT: If “Yes” | Yes AND | <SAVE | | EQG | |
| | and HR or BP or BOS is | ((HRL1=Y) | “Yes”> | | | |
| | poor, this is still | OR | \|\| 142 | | | |
| | considered an | (BPL1=Y) | | | | |
| | Emergency. If “Yes” and | OR | | | | |
| | PPs are OK, then check | (BOL1=Y)) | <SAVE | | EQG | |
| | back in 5 minutes.> \|\| | Yes AND | “Yes”>\|\| | | | |
| | Does everything look | ((HRL1=N) | 145 | | | |
| | OK? | AND | | | | |
| | | (BPL1=N) | | | | |
| | | AND | | | | |
| | | (BOL1=N) | <SAVE> \|\| 300 | | EQG | |
| | | No | | | | |
| 142 | <NRR> | | <S | | EQE | |
| | Your physiological | | “Y”>\|\|<END> | | | |
| | parameters are still poor. | | | | | |
| | I am making an | | | | | |
| | Emergency call. | | | | | |
| 145 | <NRR> | | <WAIT-300s | | | |
| | That's it for now. I will | | IS#CA-2>\|\| | | | |
| | check back in 5 minutes. | | <RETURN> | | | |
| 300 | <NRR> | | <SAVE | | EQP1 | |
| | I will call the Control | | “Yes”>\|\|END | | [Possible | |
| | Center and get them to | | | | problem | |
| | help with the situation | | | | with | |
| | | | | | equipment] | |
| 400 | <COMMENT Cardiac | | <S | | EMCS | |
| | Arrest (EWS) Emergency | | “Y”>\|\|<END> | | | |
| | Detection will be | | | | | |
| | activated. Another IS will | | | | | |
| | start communicating with | | | | | |
| | the person.>\|\| | | | | | |
| | <NO OTS> | | | | | |

CA-2 is carried out shortly after CA-1 has finished its analysis and has not found evidence of early stages of cardiac arrest. The purpose of CA-2 is to ensure that the client did not develop signs of a early stage cardiac arrest after CA-1 finished its analysis. CA-2 either performs the same procedure as CA-1, or it may just do a quick check.

The CA-2 IS follows.

a) Ask person how he/she feels.
If Bad→ED
If No Verbal Response→ED
If Lack of Understanding→ED
If OK (and poor PP gone), End
If OK (and still poor PP)→ED—Caution

Tables 45 and 46 show IS Definitions for CA-2.

TABLE 45

| | | | |
|---|---|---|---|
| IS# | CA-2 | TMT-IS Action | <CALL IS#LOS-1/ |
| | | | IU#600> |
| T-InterruptionMax | 300 | URW-IS Action | <CALL IS#LOS-1/ |
| | | | IU#700> |
| RMD-IS | 60 | NVI-IS Action | <CALL IS#LOS-1/ |
| | | | IU#800> |
| S-Time | | NUI-IS Action | <CALL IS#LOS-1/ |
| | | | IU#800> |

TABLE 46

| | | | | | | RMD- |
|---|---|---|---|---|---|---|
| | | Decision Statement | | IU | | IU |
| IU # | Output Text String | Condition | Action | Grp | IMP# | (secs) |
| 5 | <NO OTS> | | <S “Y”> | | DOCA | |
| 10 | <NAME> | Good | <SAVE> \|\| 100 | | OK1 | |
| | <N>, I'm back for a quick | Bad | <SAVE> \|\| 160 | | OK1 | |
| | health check on you. | In Between | <SAVE> \|\| 100 | | OK1 | |
| | How do you feel - Good, | | | | | |
| | Bad, In between? | | | | | |
| 100 | <COMMENT: If Client is | ((HRL1=Y) OR | 120 | | | |
| | “Good” but a PP is not | (BPL1=Y) OR | | | | |
| | good, do an Emergency- | (BOL1=Y)) | | | | |
| | Caution.. If everything | ((HRL1=N) | 140 | | | |
| | good, End.> \|\| | AND | | | | |
| | <NO OTS> | (BPL1=N) | | | | |
| | | AND | | | | |
| | | (BOL1=N) | | | | |
| 120 | <NRR> | | <SAVE | | EMC | |
| | To be on the safe side, | | “Yes”>\|\| | | [Emergency - | |
| | I'm going to Call the | | <END> | | Caution] | |
| | ERD with a Caution | | | | | |
| | Code. They will give you | | | | | |
| | a call shortly to see how | | | | | |
| | you are doing. | | | | | |
| 140 | <NRR> | | <END> | | | |
| | That's all for now. | | | | | |
| 160 | <COMMENT Cardiac | | <S | | | |
| | Arrest (EWS) Detection | | “Y”>\|\|<END> | | | |
| | will be activated. Another | | | | | |
| | IS will start | | | | | |
| | communicating with the | | | | | |
| | person.>\|\| | | | | | |
| | <NO OTS> | | | | | |

An F-1 IS is activated by M-1, after M-1 has analyzed the information it received, plus the information it gathered, and concluded that the client has fallen. The purpose of F-1 is to check if the client is in an SHE. If the client can't get up, or is unconscious, or is in some other bad condition, F-1 initiates an emergency status. If F-1 does not identify a fall-based SHE, it then activates FA-2 to start up later, such as 10 minutes later, as a follow-up. F-1 then ends.

F-1 handles all fall related trigger conditions. This includes:
Fall Detection Monitor signal
Video Monitor detects a fall
Sound Monitor detects the possible sound of a fall
Client says that he/she has fallen

An F-1 IS can include the following questions.
Did you just fall?
How are you?
   Emergency→ED
   Bad→ED
   Not sure
   OK
Can you get up?
   Yes
      Let me know when you are up.
      How are you?
         Emergency→ED
         Bad→ED
         Not sure→ED—Caution
         →Check for S/HA/CA
         →Activate F-2 to start up in 10 minutes.
         OK→ED—Caution
         →Check for S/HA/CA
         →Activate F-2 to start up in 10 minutes.
   No→ED
Are you up?
   Yes
      How do you feel?
         Emergency→ED
         Not good→ED
         Not sure→ED—Caution
         →Check for S/HA/CA
         →Activate F-2 to start up in 10 minutes.
         OK→ED—Caution
         →Check for S/HA/CA
         →Activate F-2 to start up in 10 minutes.
   No
      Let me know when you are up.
      How are you?
         Emergency→ED
         Bad→ED
         Not sure→ED—Caution
         →Check for S/HA/CA
         →Activate F-2 to start up in 10 minutes.
         OK→ED—Caution
         →Check for S/HA/CA
         →Activate F-2 to start up in 10 minutes.
      If can't get up→ED

Tables 47 and 48 show IS Definitions for F-1.

TABLE 47

| | | | |
|---|---|---|---|
| IS# | FA-1 | TMT-IS Action | <CALL IS#LOS-1/ |
| | | | IU#600> |
| T-InterruptionMax | 300 | URW-IS Action | <CALL IS#LOS-1/ |
| | | | IU#700> |
| RMD-IS | 60 | NVI-IS Action | <CALL IS#LOS-1/ |
| | | | IU#800> |
| S-Time | | NUI-IS Action | <CALL IS#LOS-1/ |
| | | | IU#800> |

TABLE 48

| | | | | | | RMD- |
|---|---|---|---|---|---|---|
| | | Decision Statement | | IU | | IU |
| IU # | Output Text String | Condition | Action | Grp | IMP# | (secs) |
| 100 | Do you think that you can get | Yes | <S “N”> \|\| 120 | | FCU | |
| | up? | No | 110 | | FCU | |
| | | Not sure | <S “NS”> \|\| 120 | | FCU | |
| 110 | OK, I will call for help for | | <S “Y”> \|\| <END> | | FCU | |
| | you. | | | | | |
| 120 | Try and get up, but don't hurt | Up | 130 | | | 120 |
| | yourself. If you have any | Can't Get Up | 200 | | | |
| | pain or other problems, just | TMT | 300 | | | |
| | stay down, and say, “I can't | | | | | |
| | get up.” And if you try and | | | | | |
| | can't get up, just say so. If | | | | | |
| | you get up, say “Up”. | | | | | |
| 130 | That's good that you're up. | OK | 140 | | | |
| | How do you feel - OK or not | Not OK | 150 | | | |
| | OK? | | | | | |
| 140 | <NRR> | | <WAIT-1800s | | | |
| | Good. | | IS#FA-2> \|\| | | | |
| | Why don't you sit down for a | | <END> | | | |
| | few minutes and rest. I will | | | | | |
| | check in with you shortly. | | | | | |
| 150 | <NRR> | | <S “Y”> \|\| <END> | | EMCS | |
| | OK, I am calling for help | | | | | |
| | right now. | | | | | |
| 200 | <NRR> | | <S “Y”> \|\| <END> | | FCU | |
| | Ok, I will call for Emergency | | | | | |
| | help. Just stay where you are | | | | | |
| | and try to be as comfortable | | | | | |
| | as possible. | | | | | |
| 300 | <NRR> | | <S “Y”> \|\| <END> | | FCU | |
| | You seem to be having | | | | | |
| | difficulty getting up. I will | | | | | |
| | call for Emergency help. Just | | | | | |
| | stay where you are and try to | | | | | |
| | be as comfortable as possible. | | | | | |

F-2 is a follow-up IS that is carried out shortly after F-1 has finished its analysis and has concluded that the situation is not an fall-based emergency, at that moment. The purpose of F-2 is to ensure that the client's condition has not gotten worse since F-1 finished. F-2 either performs the same procedure as F-1, or it may just do a quick check.

F-2 can include the following questions.
How do you feel?
   Emergency→ED
   Bad→ED
   Not sure→Check for S/HA/CA
      →Activate F-2 to start up in 30 minutes.
   OK→Check for S/HA/CA
      →Activate F-2 to start up in 30 minutes.

Tables 49 and 50 show IS Definitions for F-2.

TABLE 49

| | | | |
|---|---|---|---|
| IS# | FA-2 | TMT-IS Action | <CALL IS#LOS-1/ |
| | | | IU#600> |
| T-InterruptionMax | 300 | URW-IS Action | <CALL IS#LOS-1/ |
| | | | IU#700> |
| RMD-IS | 60 | NVI-IS Action | <CALL IS#LOS-1/ |
| | | | IU#800> |
| S-Time | | NUI-IS Action | <CALL IS#LOS-1/ |
| | | | IU#800> |

TABLE 50

| | | | | | | RMD- |
|---|---|---|---|---|---|---|
| | | Decision Statement | | IU | | IU |
| IU # | Output Text String | Condition | Action | Grp | IMP# | (secs) |
| 10 | How are you feeling now, | OK | <S “Y”> \|\| 20 | | OK1 | |
| | after your fall - Ok or not | Not Good | <S “N”> \|\| 30 | | OK1 | |
| | good? | | | | | |
| 20 | <NRR> | | <END> | | | |
| | That's good to hear. Carry | | | | | |
| | on. If any problems develop, | | | | | |
| | just call out or press the | | | | | |
| | Emergency Button. | | | | | |
| 30 | <NRR> | | <S “Y”> \|\| | | EM1 | |
| | I will make an Emergency | | <END> | | | |
| | call right now. | | | | | |

A LOS-1 IS checks for several SHEs, including unconsciousness, loss of understanding, loss of responsiveness and no verbal response. LOS-1 is triggered by any of the ISs above. The Trigger Conditions (TC) include

a) Client takes too long to reply to a question [TMT Code]

b) Client gives inappropriate words to a query [NVI Code and NUI Code]

c) Client is having trouble speaking [URW Code]

LOS-1 counts the number of times a trigger condition occurs. If trigger condition a) occurs three times in a short period of time, LOS-1 checks for unconsciousness or loss of responsiveness. If trigger condition b) occurs three times, LOS-1 checks for loss of understanding.

Tables 51 and 52 show IS Definitions for LOS-1.

TABLE 51

| | | |
|---|---|---|
| IS# | LOS-1 | TMT-IS Action |
| T-InterruptionMax | | URW-IS Action |
| RMD-IS | 60 | NVI-IS Action |
| S-Time | | NUI-IS Action |

TABLE 52

| | | | | | | RMD- |
|---|---|---|---|---|---|---|
| | | Decision Statement | | IU | | IU |
| IU # | Output Text String | Condition | Action | Grp | IMP# | (secs) |
| | <COMMENT Routine for | | | | | |
| | “No Verbal Response” - | | | | | |
| | [For handling TMT | | | | | |
| | Code]> | | | | | |
| 600 | <NO OTS> | C6=0 | C6=C6+1\|\|610 | | | |
| | | C6=1 | C6=C6+1\|\|630 | | | |
| | | C6=2 | C6=C6+1\|\|650 | | | |
| 610 | <NRR> | | <RETURN | | | |
| | John, I asked you a | | -REPEAT> | | | |
| | question over a minute | | | | | |
| | ago, and you still haven't | | | | | |
| | answered me. I will repeat | | | | | |
| | the question. | | | | | |
| 630 | <GET VALID INPUTS> | | <RETURN | | | |
| | John, I have asked you a | | -REPEAT> | | | |
| | question twice and you | | | | | |
| | still have not answered | | | | | |
| | me. Please reply with one | | | | | |
| | of the following: <>. | | | | | |
| 650 | <NO OTS> | | <S “No”>\|\| 660 | | RV | |
| 660 | Maybe you hear me but | YS2=Y | 680 | | | 20 |
| | cannot speak. I'm going | KS2=Y | 680 | | | |
| | to check it. | AW2=Y | 680 | | | |
| | If you hear me, please do | LR2=Y | 680 | | | |
| | one of the following: | TMT | 670 | | | |
| | Yelp twice, or knock | | | | | |
| | twice, or wave twice, or | | | | | |
| | lift your leg up twice. | | | | | |
| 670 | One more time: If you | YS2=Y | 680 | | | 20 |
| | hear me, please do one of | KS2=Y | 680 | | | |
| | the following: Yelp twice, | AW2=Y | 680 | | | |
| | or knock twice, or wave | LR2=Y | 680 | | | |
| | twice, or lift your leg up | TMT | 690 | | | |
| | twice. | | | | | |
| 680 | <COMMENT: Client | | 682 | | | |
| | cannot speak but can non- | | | | | |
| | verbally communicate.> \|\| | | | | | |
| | <NRR> | | | | | |
| | Thanks, I see that you can | | | | | |
| | hear me but can't talk. I | | | | | |
| | want to do a quick health | | | | | |
| | check on you. | | | | | |
| 682 | <COMMENT” Do a | | <CALL IS#S-3> | | | |
| | Stroke check on the client - | | | | | |
| | enable the client to | | | | | |
| | communicate non- | | | | | |
| | verbally. | | | | | |
| | <NO OTS> | | | | | |
| 684 | <COMMENT: Client | YS2=Y | <S “Y”>\|\|<END> | | RVS | |
| | does not show signs of | KS2=Y | <S “Y”>\|\|<END> | | RKS | |
| | Stroke, but client cannot | AW2=Y | <S “Y”>\|\|<END> | | RAW | |
| | speak. Make Emergency | LR2=Y | <S “Y”>\|\|<END> | | RLR | |
| | call.> \|\| | | | | | |
| | John, I am going to call | | | | | |
| | ERD so that they can | | | | | |
| | check in on you. \|\| <NO | | | | | |
| | OTS> | | | | | |
| 690 | <NO OTS> | MO=No | 692 | | | |
| | | MO=Yes | 694 | | | |
| | | MO=Unk | 696 | | | |
| 692 | <COMMENT Put a “Y” | | <S “Y”>\|\|<E> | | UNC | |
| | into the IMP: | | | | | |
| | {Unconscious}. This will | | | | | |
| | initiate an Emergency | | | | | |
| | Call. | | | | | |
| 694 | <COMMENT Put an “Y” | | <S “Y”>\|\|<E> | | LRM | |
| | into the IMP: {Loss of | | | | | |
| | Responsiveness}. This | | | | | |
| | will initiate an Emergency | | | | | |
| | Call. | | | | | |
| 696 | <COMMENT Put an “Y” | | <S “Y”>\|\|<E> | | LRU | |
| | into the IMP: {Loss of | | | | | |
| | Responsiveness, | | | | | |
| | movement status | | | | | |
| | unknown}. This will | | | | | |
| | initiate an Emergency | | | | | |
| | Call. | | | | | |
| | <COMMENT Routine | | | | | |
| | that is carried out when | | | | | |
| | the person is having | | | | | |
| | trouble speaking - | | | | | |
| | [For handling URW | | | | | |
| | Code]> | | | | | |
| 700 | <NO OTS> | C7=0 | C7=C7+1\|\|710 | | | |
| | | C7=1 | C7=C7+1\|\|730 | | | |
| | | C7=2 | C7=C7+1\|\|750 | | | |
| 710 | <NRR> | | <RETURN- | | | |
| | John, I didn't understand | | REPEAT> | | | |
| | some of the words that | | | | | |
| | you just spoke. Please | | | | | |
| | speak clearly. I will repeat | | | | | |
| | the question. | | | | | |
| 730 | <GET VALID INPUTS> | | <RETURN- | | | |
| | John, I still did not | | REPEAT> | | | |
| | understand some of the | | | | | |
| | words you just spoke. | | | | | |
| | Please reply with one of | | | | | |
| | the following: <>. | | | | | |
| 750 | <NRR> | | 752 | | | |
| | John, you seem to be | | | | | |
| | having problems | | | | | |
| | speaking. I want to do a | | | | | |
| | quick health check on | | | | | |
| | you. Please respond to | | | | | |
| | each question with one of | | | | | |
| | the following: Yelp twice, | | | | | |
| | or knock twice, or wave | | | | | |
| | twice, or lift your leg up | | | | | |
| | twice. | | | | | |
| 752 | <COMMENT” Do a | | <CALL IS#S-3> | | | |
| | Stroke check on the client - | | | | | |
| | enable the client to | | | | | |
| | communicate non- | | | | | |
| | verbally. | | | | | |
| | <NO OTS> | | | | | |
| 754 | <COMMENT: Client | | <SAVE “Yes”>\|\| | | TS1 | |
| | does not show signs of | | <END SESSION> | | | |
| | Stroke, but client has | | | | | |
| | trouble speaking. Make | | | | | |
| | Emergency call.> \|\| | | | | | |
| | John, I am going to call | | | | | |
| | ERD so that they can | | | | | |
| | check in on you. \|\| <NO | | | | | |
| | OTS> | | | | | |
| | <COMMENT Routine | | | | | |
| | that is carried out when | | | | | |
| | the person seems to be | | | | | |
| | confused/Has lost the | | | | | |
| | ability to understand - | | | | | |
| | [For handling NVI Code | | | | | |
| | and NUI Code]> | | | | | |
| 800 | <NO OTS> | C8=0 | C8=C8+1 \|\| 810 | | BVR | |
| | | C8=1 | C8=C8+1 \|\| 830 | | | |
| | | C8=2 | <S “Y”> \|\| 850 | | | |
| 810 | <NRR> | | <RETURN- | | | |
| | John, you didn't answer | | REPEAT> | | | |
| | my question properly. I | | | | | |
| | will repeat the question. | | | | | |
| 830 | <GET VALID INPUTS> | | <RETURN- | | | |
| | John, you still aren't | | REPEAT> | | | |
| | answering my question | | | | | |
| | properly. Please reply | | | | | |
| | with one of the following: | | | | | |
| | <>. | | | | | |
| 850 | <NRR> | | 855 | | | |
| | I want to give you a quick | | | | | |
| | memory test. | | | | | |
| 855 | What day of the week is | NI=(Day | C3=0 \|\| <S “P”>\|\| | | UT | |
| | it? | of Week) | 870 | | | |
| | | NI<>(Day | 860 | | | |
| | | of Week) | | | | |
| 860 | <COMMENT: This will | | <S “F”> \|\| <END> | | UT | |
| | initiate a Loss of | | | | | |
| | Understanding | | | | | |
| | Emergency.> \|\| | | | | | |
| | <NRR> | | | | | |
| | John, you seem to be | | | | | |
| | having problems | | | | | |
| | understanding. | | | | | |
| | I am going to notify the | | | | | |
| | ERD. | | | | | |
| 870 | | C1=1 | 880 | | | |
| | | C1><1 | 875 | | | |
| 875 | <COMMENT: Check for | | C1=1\|\|C IS#S-1> | | | |
| | Stroke.> \|\| <NO OTS> | | | | | |
| 877 | <NRR> | | 880 | | | |
| | OK, John, you seem fine. | | | | | |
| 880 | <COMMENT: Return to | | <RETURN> | | | |
| | where came from.> \|\| | | | | | |
| | <NO OTS> | | | | | |

The client's responses during the probing IS can indicate that there is a problem. The VV&I table, table 53, indicates exemplary system vocabulary.

TABLE 53

| System | | System | |
|---|---|---|---|
| Vocabulary | Recognized Spoken Words | Vocabulary | Recognized Spoken Words |
| Yes | Yes; Sure | Loss of | (Lost AND Balance): (Poor |
| | | Balance | AND Balance) |
| No | No | Loss of | (Lost AND Coordination); |
| | | Coordination | (Poor AND Coordination) |
| Pain | (In AND Pain); (Have | Left | Left |
| | AND Pain); (It AND Hurts) | | |
| Illness | (Am AND Ill); (Not AND | Right | Right |
| | Well) | | |
| Weak | (Am AND Weak) | Both | Both |
| Numbness | (Have AND Numbness) | Not Sure | Not Sure |
| Discomfort | (Have AND Discomfort) | Arm | Arm |
| Breathing | Breathing | Leg | Leg |
| Fell | (I AND Fell) | Face | Face |
| Trouble | (Trouble AND Walking) | Other | Other |
| Walking | | | |
| Chest | (My AND Chest); (Chest | Down | Down |
| | AND Problem) | | |
| Heart | (My AND Heart); (Heart | Ready | Ready |
| | AND Problem) | | |
| Can't Move | (Can't AND Move) | Steady | Steady |
| Can't Walk | (Can't AND Walk) | Not Steady | Not Steady |
| Feel Strange | (Feel AND Strange) | Pressure | Pressure |
| Feel Funny | (Feel AND Funny) | Squeezing | Squeezing |
| Something | (Something AND Wrong) | Fullness | Fullness |
| Wrong | | | |
| Don't Feel | (Don't AND Feel AND | Bad | Bad |
| Right | Right) | | |
| Nausea | Nausea; Nauseous | In Between | (In AND Between) |
| Dizzy | Dizzy; Dizziness | Done | Done |
| Lightheaded | Lightheaded | Help | Help |
| Cold Sweat | (Cold AND Sweat) | Emergency | Emergency |
| Droopy Face | (Droopy AND Face) | Up | Up |
| Droopy | (Droopy AND Mouth) | Can't Get Up | (Can't AND Get AND Up) |
| Mouth | | | |
| Headache | Headache | OK | OK |
| Good | Good | Not OK | (Not AND OK) |
| Not Good | Not Good | Somewhat | Somewhat |
| Mild | Mild | Attention | Attention |
| Moderate | Moderate | Emergency | (Emergency AND Now) |
| | | Now | |
| Serious | Serious | Trouble | (Trouble AND Walking) |
| | | Walking | |
| Severe | Severe | Trouble | (Trouble AND Speaking) |
| | | Speaking | |
| Trouble with | (Trouble AND Eyes); | Zero | Zero |
| eyes | (Trouble AND Seeing) | | |
| One | One | Point | Point |
| Two | Two | A-Z | Note: All 26 alphabets |
| Three | Three | Sunday | Sunday |
| Four | Four | Monday | Monday |
| Five | Five | Tuesday | Tuesday |
| Six | Six | Wednesday | Wednesday |
| Seven | Seven | Thursday | Thursday |
| Eight | Eight | Friday | Friday |
| Nine | Nine | Saturday | Saturday |
| Blood | (Blood AND Glucose) | Blood Oxygen | (Blood AND Oxygen AND |
| Glucose | | Saturation | Saturation) |
| Blood | (Blood AND Pressure) | Temperature | Temperature |
| Pressure | | | |
| Heart Rate | (Heart AND Rate) | Respiratory | (Respiratory AND Rate) |
| | | Rate | |
| Measurement | Measurement | | |

As noted, the client can initiate a conversation with the system. The following table 54 indicates the client initiated conditions.

TABLE 54

| | | | IMP # | CIIC |
|---|---|---|---|---|
| CII # | CII Condition Description | CII Condition | & Value | Flag |
| C20 | {Client says, “Help”} | Help | EM2 - Y | |
| C21 | | Emergency | EM1 - Y | |
| C22 | | Emergency AND Now | EMN - Y | |
| C23 | | (In AND Pain) OR | PA - Yes | |
| | | (Have AND Pain) | | |
| C24 | | Ill | IL - Yes | |
| C25 | | Not AND Well | IL - Yes | |
| C26 | | Weak | WE - Yes | |
| C27 | | Numb | NU - Yes | |
| C28 | | Discomfort | DI - Yes | |
| C29 | | Pressure | DI1 - Yes | |
| C30 | | Fullness | DI2 - Yes | |
| C40 | | Squeezing | DI3 - Yes | |
| C41 | | Feel AND Strange | FS1 - Y | |
| C42 | | Feel AND Funny | FS2 - Y | |
| C43 | | Something AND Wrong | FS3 - Y | |
| C44 | | Doesn't AND Feel AND | FS4 - Y | |
| | | Right | | |
| C45 | | Breathe | BR1 - Y | |
| C46 | | Breath | BR1 - Y | |
| C47 | | Breathing | BR1 - Y | |
| C48 | | Trouble AND Walking | TW1 - Y | |
| C49 | | Poor AND Balance | LBA - Y | |
| C50 | | Poor AND Coordination | LCO - Y | |
| C60 | | Eye AND Problem | EP - Y | |
| C61 | | Trouble AND Seeing | EP - Y | |
| C62 | | Trouble AND Speaking | TS1 | |
| C63 | | Can't AND Move | CM1 - Y | |
| C64 | | Can't AND Walk | CM2 - Y | |
| C65 | | Chest AND Problem | CH - Y | |
| C66 | | Heart AND Problem | HE - Y | |
| C67 | | Dizzy | DIZ - Y | |
| C68 | | Dizziness | DIZ - Y | |
| C69 | | Face AND Droopy | FD1 - Y | |
| C70 | | Mouth AND Droopy | FD2 - Y | |
| C71 | | Headache | HA - Y | |
| C72 | | Nauseous | NA1 - Y | |
| C73 | | Lightheaded | LH - Y | |
| C74 | | Cold AND Sweat | CS - Y | |
| C75 | | Hurts | PA - Y | |
| C76 | | I AND Fell | FA - Y | |
| C77 | | Attention | AT - Y | |
| C78 | | Ed | ED - Y | |
| C79 | | Edie | EDI - Y | |
| C80 | Client wants to know the | What AND Time | | |
| | present time. | | | |
| C81 | Client wants to know the | What AND Telephone | | |
| | telephone number for a person | AND Number AND | | |
| | or organization. | ‘Name of person or | | |
| | | organization’ | | |

Table 55 shows a table of emergency detection conditions.

TABLE 55

| | | | ED |
|---|---|---|---|
| | | | Interaction |
| | | | Session |
| EDTC | ED Condition Description | ED Condition | (IS) # |
| ST1 | Stroke Detection - | ( | ED10 |
| | {(Sudden numbness in one arm, | (((I#NUL=Arm) OR (I#NUL=Leg) | |
| | one leg, or one side of the face) | OR (I#NUL=Face)) | |
| | AND | AND | |
| | ((Problem smiling) OR (Droopy | (I#N1S=Y)) | |
| | Face/Mouth, on one side)} | AND | |
| | | ((ST1=Y) OR (ST3=Right) OR | |
| | | (ST3=Left)) | |
| | | ) | |
| ST2 | Stroke Detection - | ( | ED10 |
| | {(Sudden weakness in one arm, | ((I#WEL=Arm) OR (I#WEL=Leg) | |
| | one leg, or one side of the face) | OR (I#WEL=Face)) | |
| | AND | AND | |
| | ((Problem smiling) OR (Droopy | (I#W1S=Y)) | |
| | Face/Mouth, on one side))} | AND | |
| | | ((ST1=Y) OR (ST3=Right) OR | |
| | | (ST3=Left)) | |
| | | ) | |
| ST3 | Stroke Detection - | ( | ED10 |
| | {(In the “Arm Drift” Test, one | (AD1=Y) | |
| | arm falls faster than the other) | AND | |
| | AND | ((ST1=Y) OR (ST3=Right) OR | |
| | ((Problem smiling) OR (Droopy | (ST3=Left)) | |
| | Face/Mouth, on one side))} | ) | |
| ST4 | Stroke Detection - | ( | ED10 |
| | {(Client can't speak, or has | ((RV=N) OR (TS=Y)) | |
| | trouble speaking, but client can | AND | |
| | respond to questions non- | (((I#NUL=Arm) OR (I#NUL=Leg) | |
| | verbally - knocking; yelping, | OR (I#NUL=Face)) | |
| | waving arm or lifting led) | AND | |
| | AND | (I#N1S=Y)) | |
| | (Sudden numbness in one arm, | AND | |
| | one leg, or one side of the face) | ((ST1=Y) OR (ST3=Right) OR | |
| | AND | (ST3=Left)) | |
| | ((Problem smiling) OR (Droopy | ) | |
| | Face/Mouth, on one side)} | | |
| ST5 | Stroke Detection - | ( | ED10 |
| | {(Client can't speak, or has | ((RV=N) OR (TS=Y)) | |
| | trouble speaking, but client can | AND | |
| | respond to questions non- | ((I#WEL=Arm) OR (I#WEL=Leg) | |
| | verbally - knocking; yelping, | OR (I#WEL=Face)) | |
| | waving arm or lifting led) | AND | |
| | AND | (I#W1S=Y)) | |
| | (Sudden numbness in one arm, | AND | |
| | one leg, or one side of the face) | ((ST1=Y) OR (ST3=Right) OR | |
| | AND | (ST3=Left)) | |
| | ((Problem smiling) OR (Droopy | ) | |
| | Face/Mouth, on one side))} | | |
| ST6 | Stroke Detection - | ( | ED10 |
| | {(Client can't speak, or has | ((RV=N) OR (TS=Y)) | |
| | trouble speaking, but client can | AND | |
| | respond to questions non- | (AD1=Y) | |
| | verbally - knocking; yelping, | AND | |
| | waving arm or lifting led) | ((ST1=Y) OR (ST3=Right) OR | |
| | AND | (ST3=Left)) | |
| | (In the “Arm Drift” Test, one | ) | |
| | arm falls faster than the other) | | |
| | AND | | |
| | ((Problem smiling) OR (Droopy | | |
| | Face/Mouth, on one side))} | | |
| ST7 | Stroke-related Detection - | ( | ED10 |
| | {(While the Control Unit is | (I#DOS=Y) | |
| | checking for Stroke) AND | AND | |
| | (Control Unit detects | ((UNC=Y) OR (LRM=Y) OR | |
| | Unconsciousness OR Loss of | (LRU=Y) OR (LU=Y)) | |
| | Response OR Loss of | ) | |
| | Understanding)} | | |
| HA1 | Heart Attack Detection- | (PCC=Y) AND (PG5=Y) | ED10 |
| | {(Pain in the center of the | | |
| | chest) AND | | |
| | ((Lasts for more than 5 | | |
| | minutes) OR (Starts - Goes | | |
| | away - Comes back, for more | | |
| | than 5 minutes))} | | |
| HA2 | Heart Attack Detection- | (DCC=Y) AND (DG5=Y) | ED10 |
| | {(Discomfort in the center of | | |
| | the chest - Pressure, Fullness, or | | |
| | Squeezing) AND | | |
| | ((Lasts for more than 5 | | |
| | minutes) OR (Starts - Goes | | |
| | away - Comes back, for more | | |
| | than 5 minutes))} | | |
| HA3 | Heart Attack-related Detection - | ( | ED10 |
| | {(While the Control Unit is | (I#DOHA=Y) | |
| | checking for Heart Attack) | AND | |
| | AND (Control Unit detects | ((UNC=Y) OR (LRM=Y) OR | |
| | Unconsciousness OR Loss of | (LRU=Y) OR (LU=Y) OR | |
| | Response OR Loss of | ((RVS=Y) OR (RKS=Y) OR | |
| | Understanding OR Non-Verbal | (RAW=Y) OR (RLR=Y))) | |
| | Response Only)} | ) | |
| CAE1 | Cardiac Arrest (Early Warning | ( | ED10 |
| | Signs) Detection - | (HRL1=Y) | |
| | {(Heart Rate low) | AND | |
| | AND | ((CSNW=Y) OR (EMCS=Y) OR | |
| | ((Client says that not well) OR | (LRM=Y) OR (LRU=Y) OR | |
| | (Client says “Emergency) OR | ((BVR=Y) AND (UT=F)) OR | |
| | (Client has Loss of | ((RV=N) AND ((RVS=Y) OR | |
| | Responsiveness) OR (Client has | (RKS=Y) OR (RAW=Y) OR | |
| | Loss of Understanding) OR | (RLR=Y)))) | |
| | (Client gives no verbal | ) | |
| | response, but can give non- | | |
| | verbal response))} | | |
| CAE2 | Cardiac Arrest (Early Warning | ( | ED10 |
| | Signs) Detection - | (BPL1=Y) | |
| | {(Blood Pressure low) | AND | |
| | AND | ((CSNW=Y) OR (EMCS=Y) OR | |
| | ((Client says that not well) OR | (LRM=Y) OR (LRU=Y) OR | |
| | (Client says “Emergency) OR | ((BVR=Y) AND (UT=F)) OR | |
| | (Client has Loss of | ((RV=N) AND ((RVS=Y) OR | |
| | Responsiveness) OR (Client has | (RKS=Y) OR (RAW=Y) OR | |
| | Loss of Understanding) OR | (RLR=Y)))) | |
| | (Client gives no verbal | ) | |
| | response, but can give non- | | |
| | verbal response))} | | |
| CAE3 | Cardiac Arrest (Early Warning | ( | ED10 |
| | Signs) Detection - | (BOL1=Y) | |
| | {(Blood Oxygen Saturation | AND | |
| | low) | ((CSNW=Y) OR (EMCS=Y) OR | |
| | AND | (LRM=Y) OR (LRU=Y) OR | |
| | ((Client says that not well) OR | ((BVR=Y) AND (UT=F)) OR | |
| | (Client says “Emergency) OR | ((RV=N) AND ((RVS=Y) OR | |
| | (Client has Loss of | (RKS=Y) OR (RAW=Y) OR | |
| | Responsiveness) OR (Client has | (RLR=Y)))) | |
| | Loss of Understanding) OR | ) | |
| | (Client gives no verbal | | |
| | response, but can give non- | | |
| | verbal response))} | | |
| CAO1 | Cardiac Arrest Detection - | ( | ED10 |
| | {((Heart Rate low) | (HRL1=Y) | |
| | AND | AND | |
| | (Client is unconscious) OR | ((I#UNC=Y) OR (I#LRU=Y)) | |
| | (Client has Loss of | ) | |
| | Responsiveness, and Client's | | |
| | movement status is unknown | | |
| | because client is not in view of | | |
| | the Video Monitor)) | | |
| CAO2 | Cardiac Arrest Detection - | ( | ED10 |
| | {((Blood Pressure low) | (BPL1=Y) | |
| | AND | AND | |
| | (Client is unconscious) OR | ((I#UNC=Y) OR (I#LRU=Y)) | |
| | (Client has Loss of | ) | |
| | Responsiveness, and Client's | | |
| | movement status is unknown | | |
| | because client is not in view of | | |
| | the Video Monitor)) | | |
| CAO3 | Cardiac Arrest Detection - | ( | ED10 |
| | {((Blood Oxygen Saturation | (BOL1=Y) | |
| | low) | AND | |
| | AND | ((I#UNC=Y) OR (I#LRU=Y)) | |
| | (Client is unconscious) OR | ) | |
| | (Client has Loss of | | |
| | Responsiveness, and Client's | | |
| | movement status is unknown | | |
| | because client is not in view of | | |
| | the Video Monitor)) | | |
| FA1 | Bad Fall Detection - | ( | ED10 |
| | {(Client says that has fallen) | (FA=Y) | |
| | AND ((Client says that can't | AND | |
| | get up) OR (Client says | ((FCU=Y) OR (ESF=Y) OR | |
| | “Emergency”) OR (Client takes | (FTL=Y)) | |
| | too long to get up))} | ) | |
| FA2 | Bad Fall Detection - | ( | ED10 |
| | {(Fall Detection Monitor | (FDM=Y) | |
| | detects a fall) AND ((Client | AND | |
| | says that can't get up) OR | ((FCU=Y) OR (ESF=Y) OR | |
| | (Client says “Emergency”) OR | (RV=N) OR (FTL=Y)) | |
| | (Client not verbally responding) | ) | |
| | OR (Client takes too long to get | | |
| | up))} | | |
| FA3 | Bad Fall Detection - | ( | ED10 |
| | {(Video Monitor detects a fall) | (FAV=Y) | |
| | AND ((Client says that can't | AND | |
| | get up) OR (Client says | ((FCU=Y) OR (ESF=Y) OR | |
| | “Emergency”) OR (Client not | (RV=N) OR (FTL=Y)) | |
| | verbally responding) OR | ) | |
| | (Client takes too long to get | | |
| | up))} | | |
| FA4 | Bad Fall Detection - | ( | ED10 |
| | {(Sound of a person falling) | (FAS1=Y) | |
| | AND ((Client says that can't | AND | |
| | get up) OR (Client says | ((FCU=Y) OR (ESF=Y) OR | |
| | “Emergency”) OR (Client not | (RV=N) OR (FTL=Y)) | |
| | verbally responding) OR | ) | |
| | (Client takes too long to get | | |
| | up))} | | |
| UNC | Unconscious Detection- | ( | ED10 |
| | ((Client gives no verbal | (RV=N) AND ((RVS=N) AND | |
| | response) AND (Client gives no | (RKS=N) AND (RAW=N) AND | |
| | non-verbal response) AND (No | (RLR=N)) AND (MO=N) | |
| | movement)) | ) | |
| LRM | Loss of Responsiveness | ( | ED10 |
| | Detection - | (RV=N) AND ((RVS=N) AND | |
| | ((Client gives no verbal | (RKS=N) AND (RAW=N) AND | |
| | response) AND (Client gives no | (RLR=N)) AND (MO=Y) | |
| | non-verbal response) AND | ) | |
| | (Client is moving)) | | |
| LRU | Loss of Responsiveness | ( | ED10 |
| | Detection - | (RV=N) AND ((RVS=N) AND | |
| | ((Client gives no verbal | (RKS=N) AND (RAW=N) AND | |
| | response) AND (Client gives no | (RLR=N)) AND (MO=Y) | |
| | non-verbal response) AND | ) | |
| | (Client movement status is | | |
| | unknown - client is not in view | | |
| | of the Video Monitor)) | | |
| LU | Loss of Understanding | (BVR=Y) AND (UT=F) | ED10 |
| | Detection - | | |
| | ((Client gives inappropriate | | |
| | verbal responses) AND (Client | | |
| | fails the “Understanding” Test)) | | |
| NOV | {Client cannot speak, but can | (RV=N) AND ((RVS=Y) OR | ED10 |
| | non-verbally communicate - | (RKS=Y) OR (RAW=Y) OR | |
| | make knocking sounds; yelp; | (RLR=Y)) | |
| | wave arm; lift leg} | | |
| EMNV | {Client indicates Emergency by | EMNV=Y | ED10 |
| | non-verbal means} | | |
| EMCS | {Client indicates that the | EMCS=Y | ED10 |
| | situation is Bad or is an | | |
| | Emergency} | | |
| CM | {Client says that cannot move} | EMCM=Y | ED10 |
| CW | {Client says that cannot walk} | EMCW=Y | ED10 |
| EMN | {Client says “Emergency Now} | EMN=Y | ED10 |
| EMG | {General Emergency} | EMG=Y | ED10 |
| EMCH | {Client says needs help, and | SSF=Y | ED10 |
| | Control Unit makes Emergency | | |
| | Call} | | |
| EQP1 | {Client has equipment | EQP1=Y | ED10 |
| | problem} | | |
| ECA1 | {This is a Precaution | (EM2=Y) OR (EMC=Y) | ED10 |
| | Emergency Call} | | |
| EM5 | {Control Unit decides to make | I#EM5=Y | Ed10 |
| | an Emergency call.} | | |
| PACW | {Client in severe pain, and can't | PACW=Y | ED10 |
| | walk; can't call for help} | | |
| ILCW | {Client has severe illness, and | ILCW=Y | ED10 |
| | can't walk; can't call for help} | | |
| WECW | {Client is severely weak, and | WECW=Y | ED10 |
| | can't walk; can't call for help} | | |
| TS | {Client had trouble speaking} | (I#TS2=Y) AND (I#EM5=Y) | ED10 |
| BD1 | {(Client has breathing | (BD=Y) AND ((EM1=Y) OR | ED10 |
| | difficulties) AND ((Client says | (EMNV=Y)) | |
| | that feels that it is an | | |
| | Emergency)) OR (Non-verbally | | |
| | indicates that it is an | | |
| | Emergency)} | | |
| ST1 | {(Client says that feels | (FSB=Y) AND (EM1=Y) | ED10 |
| | “strange”) AND (Client says | | |
| | that it is an Emergency)} | | |
| ENV1 | {(Client makes the special | (S#EMK=Y) AND (S#SY=Y) | ED10 |
| | Emergency knocking sound - 2 | | |
| | knocks-pause-2 knocks) AND | | |
| | (Client confirms this with a | | |
| | knock, when asked to confirm)} | | |
| ENV2 | {(Client makes the special | (S#EMY=Y) AND (S#SY=Y) | ED10 |
| | Emergency yelping sound - 2 | | |
| | yelps-pause-2 yelps) AND | | |
| | (Client confirms this with a | | |
| | yelp, when asked to confirm)} | | |
| ENV3 | {(Client makes the special | (V#EMW=Y) AND (V#VY=Y) | ED10 |
| | Emergency arm wave - 2 | | |
| | waves-pause-2 waves) AND | | |
| | (Client confirms this with a | | |
| | wave, when asked to confirm)} | | |
| ENV4 | {(Client makes the special | (V#EML=Y) AND (V#VY=Y) | ED10 |
| | Emergency leg lift - 2 lifts- | | |
| | pause-2 lifts) AND (Client | | |
| | confirms this with a leg lift, | | |
| | when asked to confirm)} | | |

In table 55, only columns 1, 3 and 4 may be put into the actual ED table. All ED conditions assume that the client is within communication range of the control device.

In one embodiment, a system that a client has in his home or carries around with him includes all of the data contained in an IDS store, a PT table, an RT table, a CIIC table and a VV&I table, plus defined IMPs. This may be considered a basic unit. In another embodiment, the system can include the features of the basic unit, plus a microphone and speaker. In another embodiment, the system includes the features of the basic unit, plus a microphone and speaker and monitoring devices, such as physiological monitors. A system with monitoring devices can use the parameter values received from the monitoring devices as triggers to initiate a probing conversation of the client's status, as well as to determine whether an emergency is occurring or about to occur.

In some embodiments, the system includes all of the features of the basic unit, plus a microphone and speaker, physiological monitoring devices, and a sound monitoring device and/or an image monitoring device. The system can use the sound monitoring device to detect and confirm that the client needs assistance. For example, the system can be programmed to recognize successive yelps or knocks as a sign from the client that he is in an emergency situation. The system can probe to confirm the client's need for help and auto-alert emergency response personnel. Further, the system can be programmed to accept 1 or 2 yelps/knocks as Yes/No replies to verbal questions. If the system includes optional image recognition capabilities, the system can be programmed to recognize three successive hand waves or leg waves as a sign from the client that they are in an emergency situation. The system will then probe to confirm the emergency situation and auto-alert emergency response personnel, if necessary. Further, the system can accept 1 or 2 hand waves/leg waves as Yes/No replies to verbal questions.

In some embodiments, the system includes all of the features of the basic unit, plus a microphone and speaker and a user input device with a screen. The client can also use the user input device with the screen without the microphone and speaker or can listen to the verbal questions from the speaker and respond using the input device. The system can initiate a conversation with the client, by either speaking to the client or displaying a question on the screen.

In some embodiments, the system is a mobile system including a base unit, where the base unit includes all of the features of the basic unit, a microprocessor, memory, an OS, a GPS locator, and an ability to run custom software, such as software that communicates with a mobile phone, which can dial for help, a wireless transceiver. An optional communicator device can plug into the base unit or communicate wirelessly with the base unit. The communicator can be attached to the client's clothing, such as pinned to the client's shirt or blouse. It can be attached to a neck chain and worn around the neck. The base unit can alternatively be a mobile phone that includes the features described in the base unit above and which auto-dials and/or auto-receives calls through an cell phone sub-system. Optionally, the mobile system also is able to communicate with on-person or in-person physiological monitors. In some implementations, the mobile system can communicate with a sound monitoring system. In some implementations, the mobile system includes a user input device, such as device built into a phone.

Because the system is able to verbally interact with the client, the system can be used for disease management assistance, such as to help a client who is attempting to manage the causes of symptoms of his disease at home. Such disease management may include a program where the client may take specific medication (specific dosage) at specific times, measure various health-related parameters, such as his blood glucose level, blood pressure or weight, adjust program activities, or other activities, based on the measurements. record various health-related measurements, provide the measurement to a health care provider, regularly visit his health care provider, recording what was done, and when, such as taking medication, exercising, and eating, or become informed about the chronic disease.

Unfortunately, the person may have trouble following a program due to being forgetful, lacking motivation or having mental impairment, such as some dementia (Alzheimer's) or depression. The system can automatically remind, query and record information related to the program related activities and forward the information to a health care provider. Because the system described herein interacts with the client using conversation based interaction, the client is more likely to be receptive to the assistance provided.

The system can use the verbal interaction capability to interact with a client, to help with such disease management activities as: reminders, compliance checking, and health-related data gathering. In addition, the client can wear a wireless on-person communicator as they go about their daily activities. This enables the apparatus to communicate with the client at any time. All the decision-making and processing associated with disease management assistance is done solely by the system that is local to the client, that is in the client's home or on the client's person, no connection is required to a remote central computer. The system can perform the following functions in disease management mode

1) Verbal Reminders

At a specific time/date, verbally give a reminder

The system can wrap the reminder with a mini-conversation

The system can first ensure that the person is listening, then speak the reminder, then confirm that the person has properly heard the reminder

If not, can repeat the reminder, or give info associated with the reminder

The system can be used to provide daily medication reminders, reminders to do exercise, or to call someone

2) Obtain information on a person's health status (daily or otherwise)

At a certain time, request that the person provides her health status

The system leads the person through a list of activities designed to obtain health parameters, including:

If a personal monitoring device is connected to the system, such as a blood pressure monitor, the system instructs the person to use the monitor, the measurement is automatically saved in memory.

If part of the program is for the person to measure something with a stand alone monitor, the system can instruct the person to go to the monitor, or bring the monitor to the system, use the monitor, and then to verbally provide the reading to the system.

The system can verbally interact with the person to obtain other health related information, such as: “Did you have a good sleep?”, or “Rate the pain you have in your lower back today.”

3) Compliance Checking Through computer verbal interaction

The system can ask one or more daily questions to find out if the person has complied with various aspects of his/her disease management program, for example, “Did you take your pills at 9 a.m.?”, or “Did you take your daily 30 minute walk today?”

In addition, if the person did not comply with something, the system can ask the person to identify why not; e.g., too tired; too cold outside.

4) Information Providing Through Computer Verbal Interaction

The system can verbally provide information to person, upon request, for example, the person may ask, “What is atrial defibrillation?”, and the system can provide a short verbal interaction. Or, the person may ask, “Is it OK for me to eat white bread?”

The system can also have other capabilities, such as the system being easily customizable for every user. The system can be easily customized for every user, for example, reminders can be create to occur at specific times, with information specific to the user. The client's system can be configured under the control of a person's health care provider or by a health care provider. The system can be remotely configured, such as to modify the system. The system can easily conveniently gather information whenever required, such as health status at anytime of the day or night. Further, system can gather health status for as long as required. Once the information is gathered, it can be forwarded to emergency personnel. If the personnel have been called to an emergency for one of our client's, they can be automatically provided with the client's current and recent past history information before arriving to the client's home. Additional information can be provided, such as the client's nearest relative/friend contact info, and various other medical information. Also, an additional method of obtaining the latest client information can be a query, such as by a button on the unit, that can automatically engage a conversation with the EMS personnel or to wireless provided the information to an emergency services mobile computer. The system can act as a verbal pain button, that is, allowing the client to verbally indicate when he or she is experiencing pain. The system can offer an optional handheld user input unit with a screen. Further, the system can support other virtual computer based interaction applications, other than SHE monitoring. The system can be configured to initiate conversations that are game-like in nature to help exercise the client's mental facilities and to also monitor any potential mental medical emergency. It can also be used to track any long term changes in mental acuity.

The client's physical activity can also be monitored as it relates to his/her physiological parameters. For example, the system can instruct the client to exercise in one spot (arm movements, leg movements, etc.) and continually measures the client's heart rate (oxygenation level, breathing rate, etc.) to ensure it achieves a minimum rate for a minimum duration and to immediately tell the client to stop if the heart rate exceeds a maximum level. This information can also be provided by the client's physician and can act as a prescription of exercise by the physician.

The systems described herein can provide health monitoring. However, the system could also be used to monitor a person who is young or somewhat mentally incapacitated. Thus, the system could be used in a babysitting mode, such as for children who are old enough to be on their own, but where the parents still want to be reassured of the child's safety. Such a system could periodically or randomly ask the child a question, such as, “What is your middle name?” or “Are you OK?” to make sure that the child is home and does not need assistance. If the child responds with the wrong answer, says that he or she is not OK, or does not respond at all, the system can call someone for assistance. As with the health monitoring systems, the system can call emergency services or a central center or the system can call someone from a list of contacts, such as in a database that lists information about the person being monitored or the address at which they system is located. Alternatively, the system can ask the person being monitored for a name or number of someone who should be called if there is a problem.

A number of embodiments of the invention have been described. Nevertheless, it will be understood that various modifications may be made without departing from the spirit and scope of the invention. For example, any of the interactions described herein can take place through the system's speakers and microphone or through the user input device. Accordingly, other embodiments are within the scope of the following claims.

1-229. (canceled)

230. A system for monitoring a subject, comprising:
a microphone;
a speaker;
a speech recognition system;
a speech synthesizer;
a quality of responsiveness determiner; and
a processor configured to:
initiate computer generated verbal interaction with the subject, including selecting speech for the speech synthesizer to synthesize and cause to be generated by the speaker, wherein the verbal interaction with the subject is selected to elicit a verbal response from the subject;
receive digitized sound representing verbal responses from the subject and captured by the microphone;
cause the speech recognition system to perform speech recognition on the digitized sound;
cause the quality of responsiveness determiner to determine a quality of responsiveness of the subject to the synthesized speech; and
determine whether to contact a predetermined contact for the subject after the quality of the responsiveness is determined.

231. The system of claim 230, wherein determining whether to contact a predetermined contact for the subject includes basing the determination on the quality of the responsiveness.

232. The system of claim 231, wherein the quality of responsiveness is one of delayed, valid or invalid.

233. (canceled)

234. The system of claim 232, wherein when a valid response is determined, the processor is further configured to:
determine whether the valid response indicates that the subject is experiencing a health problem; and
if the subject is experiencing a health problem, cause emergency services to be contacted.

235. (canceled)

236. The system of claim 232, wherein the processor is configured to determine to contact a predetermined contact when the quality of responsiveness is delayed or invalid.

237. The system of claim 230, wherein initiating the computer generated verbal interaction initiates a script of questions related to detecting a heart attack or a stroke.

238. The system of claim 230, wherein the processor is configured to:
after determining with a computer the quality of the responsiveness, generate additional synthesized speech to elicit a further verbal response from the subject, wherein the additional synthesized speech poses a question to the subject regarding a safety or health status of the subject;
receive a response to the question regarding the safety or health status of subject; and
perform speech recognition on the response to generate corresponding subsequent text, wherein determining whether to contact a predetermined contact is based on the subsequent text.

239-252. (canceled)

253. A personal emergency monitoring system, comprising:
a) a speaker that outputs audio signal, including computer generated speech, to a subject
b) a microphone that receives sounds, including speech of the subject, including speech related to responses to questions and statements, and speech related to keywords and keyword phrases initiated by the subject, and converts the sounds into an input audio signal; and
c) a control unit, comprising:
i) a computer
ii) an audio interface that receives the input audio signal from the microphone and sends the output audio signal to the speaker;
iii) a speech synthesis module that reviews speech-related text, including questions and statements, and converts the text into an output audio signal, and sends the signal to the audio interface;
vi) a speech recognition module that receives the input audio signal from the audio interface, and
converts the input audio signal into corresponding speech-related text, including responses, keywords and phrases and
indicates if the input audio signal contains speech that is unrecognizable;
v) a data storage area for storing data, including data associated with
said responses from the subject
and keywords and phrases;
vi) a quality of responsiveness interpreter that determines a quality of responsiveness associated with every received processed response or anticipated response, from the subject, said quality of responsiveness comprising a valid response or poor quality of responsiveness;
vii) a verbal input handling module that is configured to:
receive said responses, keywords and phrases from the speech recognition module
process each received response and received keyword and phrase
receive the quality of responsiveness associated with every received processed response or anticipated response, from the quality of responsiveness interpreter, and
pass the quality of responsiveness and the processed response, if it is a valid response, to an input processing module
viii) an interaction session definition store that contains one or more interaction session definitions, each interaction session definition defining a unique verbal interaction between the system and the subject and comprising:
statements and questions to be spoken to the subject,
possible valid responses, if any, associated with each statement and question
data, if any, to be saved in the data storage area, for each possible valid response
a next action to be carried out, including a next statement or question to be spoken, for each possible valid response and every type of possible quality of responsiveness
ix) the input processing module, which is configured to execute the interaction session definitions, execution carried out upon receiving an instruction to execute a particular interaction session definition, execution of an interaction session definition comprising:
outputting a statement or question, comprising speech-related text, to the speech synthesis module
receiving a valid processed response, if any, and quality of responsiveness
saving data, if any, associated with the valid processed response in the data storage area
determining the next action to be carried out, and carrying the next action out
repeating the steps of outputting, receiving, storing data and determining until execution of the interaction session definition is complete
x) an emergency condition table that contains one or more emergency situation logic expressions, each emergency situation logic expression representing a possible emergency situation that may be experienced by the subject; and
xi) an emergency analysis module that evaluates each of the emergency situation logic expressions repeatedly and on an on-going basis,
upon evaluating a true said emergency situation logic expression, determining that the subject is experiencing a possible emergency situation, and initiating emergency alerting action.

254. The system of claim 253, further comprising:
a) a heart attack interaction session definition that when executed by the input processing module causes the system to verbally interact with the subject, verbal interaction comprising:
asking the subject one or more heart attack related questions, including questions based on the early warning signs of heart attack as promoted by leading public health organizations
receiving response from the subject
saving the data associated with the response in the data storage area, wherein
execution is based on analysis carried out when the input processing module executes an M-1 interaction session definition;
b) a stroke interaction session definition that when executed by the input processing module causes the system to verbally interact with the subject, verbal interaction comprising:
asking the subject one or more stroke related questions, including questions based on the early warning signs of stroke as promoted by leading public health organizations
receiving response from the subject
asking the subject to carry out one or more actions, each action related to identifying if the subject has a stroke-related early warning signs, including actions based on paramedic guidelines, and asking the subject to indicate the outcome of an action
receiving a verbal indicator from the subject
saving data associated with each of the responses and verbal indicators in the data storage area, wherein
execution is based on analysis carried out when the input processing module executes the M-1 interaction session definition
c) a LOS interaction session definition that when executed by the input processing module causes the system to verbally interact with the subject, verbal interaction comprising:
causing the input processing module to repeat a last question or statement
tracking a number of occurrences of quality of responsiveness during execution of an interaction session definition
tracking a number of occurrences of too-much-time quality of responsiveness associated with the same question or statement
if several occurrences of too-much-time quality of responsiveness associated with the same question or statement are detected, setting a loss of understanding flag in the data storage area
if several occurrences of poor quality of responsiveness are detected during the execution of the LOS interaction session definition, verbally testing the subject for loss of understanding
if the subject fails a loss of understanding test, setting the loss of understanding flag in the data storage area, wherein
execution based on determination by the quality of responsiveness interpreter of poor quality of responsiveness during any or all verbal interaction between the system and the subject
d) the M-1 interaction session definition that when executed by the input processing module causes the system to verbally interact with the subject, verbal interaction comprising:
i) asking the subject one or more initial probing questions based on a true probing trigger logic expression that initiates execution of the M-1 interaction session definition, receiving a response from the subject saving data, if any, associated with valid response, into the data storage area,
analyzing the saved data associated with the initial probing questions and the probing trigger logic expression to determine whether to start up one or more probing interaction session definitions and in what order, and starting them up, if any, wherein
execution of this verbal interaction carried out based on the trigger condition analysis module evaluating a true said probing trigger logic expression that is associated with a system-recognized emergency situations, including heart attack and stroke
ii) initiating a verbal instruction with the subject to confirm that an emergency situation exists, receiving the response from the subject, saving data associated with the response into the data storage area, wherein
execution of this verbal interaction is carried out based on the trigger condition analysis module evaluating a true said probing trigger logic expression that is associated with a subject-initiated emergency-related keyword or phrase
iii) initiating a verbal instruction with the subject if the subject feels that is the subject is in an emergency situation, receiving the subject's response,
if the response is affirmative, setting a general emergency flag in the data storage area, wherein
execution of this verbal interaction is carried out when no specific type of emergency situation is determined,
wherein execution of a master interaction session definition is carried out by the input processing module based on the trigger condition analysis module evaluating a true said probing trigger logic expression that is associated with one of the system-recognized emergency situations, including heart attack and stroke, or with a health issue
e) said emergency situation logic expressions including at least one of:
one, several or no logic expressions representing possible heart attack related emergency situations, each possible heart attack related emergency situation based on the early warning signs of heart attack, including the early warning signs contained in a standardized list of early warning signs of heart attack as promoted by leading public health organizations
one, several or no logic expressions representing possible stroke-related emergency situations, each possible stroke related emergency situation based on the early warning signs of stroke, including the early warning signs contained in a standardized list of early warning signs of stroke as promoted by leading public health organizations, and the early warning signs as utilized by paramedics and other emergency response people to help identify people in a stroke related emergency situation
one, several or no logic expressions representing a loss of understanding emergency situation
one, several or no logic expressions representing a loss of responsiveness emergency situation
one, several or no logic expressions representing an emergency situation verbally indicated by the subject, a verbal indicator matching a pre-defined keyword or keyword phrase
one, several or no logic expressions representing a general emergency situation
f) said quality of responsiveness, including a valid response, non-valid response, un-recognized word, non-understood word, or too much time; and
g) said emergency alerting action including alerting a pre-defined contact, or alerting the subject.

255. The system of claim 254, further comprising:
a) a routine trigger table that contains one or more routine trigger logic expressions, each routine trigger logic expression representing an event that, when the event occurs, results in the start up of a routine check interaction session definition
b) a probing trigger table that contains one or more probing trigger logic expressions, each probing trigger logic expression representing an event that, when the event occurs, results in a start up of one of the interaction session definitions
c) a trigger condition analysis module that continuously evaluates each of the routine trigger logic expressions and probing trigger logic expressions, repeatedly and on an on-going basis,
upon determining a true routine trigger logic expression, causing the input processing module to execute the routine check interaction session definition,
upon determining a true probing trigger logic expression, causing the input processing module to execute the interaction session definition that is associated with the true probing trigger logic expression
d) a client-initiated interaction condition table that contains pre-defined keywords and keyword phrases, each pre-defined keyword and keyword phrase associated with data for saving into the data storage area
e) the verbal input processing module being further configured to:
check if a received word or phrase is one of the pre-defined keywords or keyword phrases,
save the data associated with the pre-defined keyword or keyword phrase into the data storage area, if the received word or phrase is one of the pre-defined keywords or keyword phrases
f) a routine check interaction session definition that when executed by the input processing module causes the system to verbally interact with the subject, the verbal interaction comprising:
asking several questions dealing with general health issues
receiving responses from the subject
saving data associated with the responses in the data storage area, wherein
execution is based on the trigger condition analysis module determining a true routine trigger logic expression
g) a requested interaction session handling module that determines which interaction session definition the input processing module is to execute, and when, based on
received interaction session definition start up requests from the trigger condition analysis module, and
the priority of the interaction session definition being requested for start up; and
h) a telecommunications interface.

256. The system of claim 255, wherein:
a) said routine trigger logic expressions represents at least one of:
said system has had no verbal interaction with the subject for a certain period of time
said system has not heard the subject speak for a certain period of time, or
the present time is a pre-defined time
b) said probing trigger logic expressions representing at least one of:
said subject verbally confirms that the subject has a possible early warning sign of stroke
said subject verbally confirms that the subject has a possible early warning sign of heart attack
said subject verbally confirms that the subject has a general health issue
c) said pre-defined keyword or keyword phrases are associated with at least one of:
possible early warning signs of heart attack,
possible early warning signs of stroke,
possible health issues,
possible general emergency indicators
d) said general health issues including at least one of pain, illness, or weakness
e) said loss of understanding test comprising:
asking the subject one or more questions that the subject normally knows the answer to
if the subject does not provide a valid response after the question or questions has been repeated several times, setting the loss of understanding flag in the data storage area.

257. The system of claim 256, wherein
a) the speaker and the control unit communicate wirelessly; and
b) the microphone and the control unit communicate wirelessly

258. The system of claim 257, wherein
a) the speaker is portable; and
b) the microphone is portable.

259. The system of claim 258, wherein
the control unit is portable, and includes wireless telecommunications capabilities.

260. The system of claim 254, wherein
the probing interaction session definitions can be started by probing triggers.

261. The system of claim 253, wherein:
a) the microphone is configured to pick non-verbal sounds made by the subject including sound-encoded responses and environmental sounds
b) the control unit, further comprising:
i) a sound recognition module that
receives the input audio signal picked up by the microphone,
analyses the input audio signal, looking for pre-defined sounds,
upon detecting one of these said pre-defined sounds, saving data associated with the pre-defined sound into the data storage area, and associated timestamp
ii) the data storage area, further storing data associated with said pre-defined sounds
iii) said interaction session definitions further comprising:
special conditions, including special conditions that include sound-encoded responses, and associated actions including the next question or statement to be spoken
iv) said input processing module further configured to:
process said special conditions, including said sound-encoded responses, and carry out actions associated with the special conditions, including sending out the next question or statement
v) the probing trigger table, further including:
probing trigger logic expression representing events that include detection of said pre-defined sounds
probing trigger logic expression representing events that include detection of said sound-encoded responses
vi) an MS-1 interaction session definition that when executed by the input processing module causes the system to interact with the subject, verbal interaction comprising:
vi-1) asking the subject to confirm the occurrence of the detected said pre-defined sound
receiving a response from the subject
saving data associated with the response in the data storage area, wherein the response can be a verbal response or a sound-encoded response, wherein
execution of this asking is carried out based on the trigger condition analysis module evaluating a true sound-based probing trigger logic expression
vi-2) asking the subject to confirm that an emergency situation exists,
if receive a verbal confirmation, setting an emergency flag
if receive sound-encoded confirmation, setting the emergency flag, wherein execution of this asking is carried out based on the trigger condition analysis module evaluating a true sound-based probing trigger logic expression representing a sound-encoded emergency indicator, wherein
execution of the MS-1 interaction session definition is carried out by the input processing module based on the trigger condition analysis module evaluating a true sound-based probing trigger logic expression
vii) said M-1 interaction session definition further executed by the input processing module based on the MS-1 interaction session definition determining that the subject has confirmed verbal or by sound-encoded response the occurrence of a pre-defined sound; and
viii) said emergency condition table, further comprising:
an emergency situation logic expression representing an emergency situation indicated by a set emergency flag
an emergency situation logic expression representing an emergency situation verified by a sound-encoded response.

262. The system of claim 261, wherein:
a) said pre-defined sounds including at least one of:
a cry of pain
a sound-encoded emergency indicator
a breaking
a falling sound
b) said probing triggers, contained in probing trigger table, further including at least one of:
a sound-based probing trigger logic expression representing an event comprising detecting the sound of someone falling; starts up a MS-1 interaction session definition
a sound-based probing trigger logic expression representing an event comprising of detecting the sound of glass breaking; starts up a MS-1 interaction session definition
a sound-based probing trigger logic expression representing an event comprising of detecting a sound-encoded emergency indicator; starts up a MS-1 interaction session definition
a probing trigger logic expression representing an event comprising of detecting a sound-encoded confirmation of pain; starts up a M-1 interaction session definition
a probing trigger logic expression representing an event comprising of detecting a sound-encoded confirmation of falling; starts up a M-1 interaction session definition.

263. The system of claim 253, further comprising:
a) a heart rate monitoring device, sending subject-measured heart rates to the control unit
b) said control unit, further comprising:
i) a heart rate monitor device driver that:
receives heart rate measurements from the heart rate monitoring device
saves the received heart rate measurements in the data storage area with a timestamp
determines a range that each heart rate measurement is in and saves this data in the data storage area with a timestamp
ii) the data storage area further storing data associated with heart rate measurements from the heart rate monitoring device
iii) a cardiac arrest interaction session definition that when executed by the input processing module causes the system to verbally interact with the subject, verbal interaction comprising:
asking the subject to confirm that an emergency situation exists, receiving a response from the subject, saving data associated with response into said data storage area
if the response is affirmative, and setting a cardiac arrest flag, wherein
execution is based on analysis carried out when the input processing module executes the M-1 interaction session definition
iv) the probing trigger table, further including:
probing trigger logic expressions representing low or very low heart rate
v) said emergency condition table, further comprising:
emergency situation logic expression representing a set emergency flag and a very low heart rate measurement
vi) the M-1 interaction session definition, further comprising
starting up the cardiac arrest interaction session definition when low heart rate detected; and
vii) an MPP-1 interaction session definition that when executed by the input processing module causes the said system to interact with the subject, verbal interaction comprising:
asking the subject to confirm that an emergency situation exists,
if a verbal confirmation is received, setting a confirmed emergency flag
execution of this verbal interaction is carried out based on the trigger condition analysis module evaluating a true probing trigger logic expression representing a very low heart rate.

264. The system of claim 253, further comprising:
a) a video camera that records images; and transmits a video signal to the control unit
b) the control unit, further comprising:
i) an image recognition module that
receives the video signal,
analyses the video signal, looking for pre-defined images,
upon detecting one of said pre-defined images, saving data associated with the pre-defined image into said data storage area
ii) the data storage area, further storing data associated with said pre-defined images
iii) said interaction session definitions further comprising:
special conditions, including special conditions that include image-encoded responses, and associated actions including the next question or statement to be spoken
iv) said input processing module further comprising:
processing of said special conditions, including said image-encoded responses, and carrying out actions associated with the special conditions, including sending out the next question or statement
v) the probing trigger table, further including:
probing trigger logic expression representing events that include the detection of said pre-defined images
probing trigger logic expression representing events that include the detection of said image-encoded responses
vi) an MV-1 interaction session definition that when executed by the input processing module causes the system to interact with the subject, in interaction comprising:
vi-1) asking the subject to confirm the occurrence of the detected said pre-defined image,
receiving a response from the subject,
saving data associated with the response in the data storage area; wherein the response can be a verbal response or an image-encoded response
execution of this interaction is carried out based on the trigger condition analysis module evaluating a true image-based probing trigger logic expression
vi-2)—asking the subject to confirm that an emergency situation exists,
if a verbal confirmation is received, setting an emergency flag
if an image-encoded confirmation is received, setting an emergency flag
execution of this asking is carried out based on the trigger condition analysis module evaluating a true image-based probing trigger logic expression representing an image-encoded emergency indicator
execution of the MV-1 interaction session definition is carried out by the input processing module based on the trigger condition analysis module evaluating a true image-based trigger logic expression
vii) said M-1 interaction session definition further executed by the input processing module based on the MV-1 interaction session definition determining that the subject has confirmed verbally or by image-encoded response the occurrence of a pre-defined image and motion
viii) said emergency condition table, further comprising:
an emergency situation logic expression representing an emergency situation verified by an image-encoded response.

265. The system of claim 264, wherein:
a) said pre-defined images include at least one of:
image-encoded “Yes” and “No”
client falling
client stumbling
face/mouth droopy
image-encoded emergency indicator
b) said probing triggers contained in probing trigger table, further including at least one of:
an image-based probing trigger logic expression representing an event comprising of detecting someone falling; which starts up MV-1 interaction session definition
probing trigger logic expression representing an event comprising of detecting an image-encoded confirmation of pain; which starts up M-1 interaction session definition.

266. The system of claim 253, further comprising:
a) one or several monitoring devices, including at least one of:
physiological parameter monitoring device
safety monitoring device, including a fall detection device
video or image camera; or
environmental monitoring devices, including motion detector devices
b) the control unit, further comprising:
i) communication means for receiving data from any and all of the monitoring devices
ii) the data store area further configured to store data from the monitoring device or devices
iii) a device driver for every monitoring device, each device driver configured to:
accept information from the associated monitoring device
save data, based on the information into the data storage area, and timestamp
iv) said interaction session definitions further comprising:
special conditions, including special conditions that include information received from said monitoring devices
v) said input processing module further comprising:
processing of said special conditions, and carrying out actions associated with the said-special conditions, including sending out the next question or statement
vi) the probing trigger table, further including:
probing trigger logic expressions representing events that include information associated with the monitoring devices.

267. The system of claim 266, further comprising:
i) said emergency condition table further containing one or more emergency situation logic expressions that include information associated with the monitoring devices.
